SEQUENCE LISTING (291 sequences)

SEQ ID NO: 1 - protein, 5 aa, human
RNAIS

SEQ ID NO: 2 - protein, 17 aa, human
GIIAIFGTANYAQKFQG

SEQ ID NO: 3 - protein, 11 aa, human
GPNYYENFFDY

SEQ ID NO: 4 - protein, 11 aa, human
RASQDIANSLA

SEQ ID NO: 5 - protein, 7 aa, human
GASRLEG

SEQ ID NO: 6 - protein, 9 aa, human
QQYFSFPRT

SEQ ID NO: 7 - protein, 5 aa, human
KNAIS

SEQ ID NO: 8 - protein, 17 aa, human
GISAIFGTANYAQKFQG

SEQ ID NO: 9 - protein, 15 aa, human
HPTYHFDKSGYRFDS

SEQ ID NO: 10 - protein, 13 aa, human
SGSSSNIGNNYVS

SEQ ID NO: 11 - protein, 7 aa, human
DNNKRPS

SEQ ID NO: 12 - protein, 11 aa, human
GTWDSSLSAGV

SEQ ID NO: 13 - protein, 8 aa, human
SFSTYTIS

SEQ ID NO: 14 - protein, 17 aa, human
RSIPLLGITNYAQKFQG

SEQ ID NO: 15 - protein, 14 aa, human
YQSSDYYNSEYFQH

SEQ ID NO: 16 - protein, 12 aa, human
RASQNVGTNYLA

SEQ ID NO: 17 - protein, 7 aa, human
GASSRAT

SEQ ID NO: 18 - protein, 9 aa, human
QQYSSSPLT

SEQ ID NO: 19 - protein, 5 aa, human
KNAIS

SEQ ID NO: 20 - protein, 17 aa, human
GITAIFGTPNYAQKFQG

SEQ ID NO: 21 - protein, 11 aa, human
SGTYYVSYLDS

SEQ ID NO: 22 - protein, 12 aa, human
RASQSVSSSYLA

SEQ ID NO: 23 - protein, 7 aa, human
GASSRAT

SEQ ID NO: 24 - protein, 9 aa, human
QQYGSSPLT

SEQ ID NO: 25 - protein, 5 aa, human
AYGIS

SEQ ID NO: 26 - protein, 17 aa, human
GIVALFGTTNYAQKLQG

SEQ ID NO: 27 - protein, 11 aa, human
SGSYYPDYFQY

SEQ ID NO: 28 - protein, 12 aa, human
RASQSVSSSYLA

SEQ ID NO: 29 - protein, 7 aa, human
GASSRAT

SEQ ID NO: 30 - protein, 9 aa, human
QQYGSSPRT

SEQ ID NO: 31 - protein, 5 aa, human
KYAIT

SEQ ID NO: 32 - protein, 17 aa, human
GIIAIFGSTNYAQKFQG

SEQ ID NO: 33 - protein, 11 aa, human
GPHYYESHLDY

SEQ ID NO: 34 - protein, 12 aa, human
RASQGMSSSFLA

SEQ ID NO: 35 - protein, 7 aa, human
GASTRAT

SEQ ID NO: 36 - protein, 9 aa, human
QQYGSSPRT

SEQ ID NO: 37 - protein, 5 aa, human
RSAIS

SEQ ID NO: 38 - protein, 17 aa, human
GILAIFGTTKYAQKFQG

SEQ ID NO: 39 - protein, 11 aa, human
GPHYYVSYFDS

SEQ ID NO: 40 - protein, 11 aa, human
RASQSVRSYLA

SEQ ID NO: 41 - protein, 7 aa, human
DASNRAT

SEQ ID NO: 42 - protein, 9 aa, human
QQRSNWLFT

SEQ ID NO: 43 - protein, 5 aa, human
SNAVS

SEQ ID NO: 44 - protein, 17 aa, human
GIIAIFGTPKYAQKFQG

SEQ ID NO: 45 - protein, 11 aa, human
GPNYYESYFDY

SEQ ID NO: 46 - protein, 11 aa, human
RASQGISNYLA

SEQ ID NO: 47 - protein, 7 aa, human
GASTLHS

SEQ ID NO: 48 - protein, 9 aa, human
QKYHSAPLT

SEQ ID NO: 49 - protein, 5 aa, human
KYGIS

SEQ ID NO: 50 - protein, 17 aa, human
GILAIFGTTNYAQKFQG

SEQ ID NO: 51 - protein, 11 aa, human
GNTYYSSYFDQ

SEQ ID NO: 52 - protein, 11 aa, human
GGDKIGTGPVH

SEQ ID NO: 53 - protein, 7 aa, human
ASTHRPS

SEQ ID NO: 54 - protein, 12 aa, human
QMWDSSSVHPIV

SEQ ID NO: 55 - protein, 5 aa, human
KNAIS

SEQ ID NO: 56 - protein, 17 aa, human
GIIAIFGTANYAQKFQG

SEQ ID NO: 57 - protein, 11 aa, human
GPNYYENYFDF

SEQ ID NO: 58 - protein, 11 aa, human
RTSQAITNYLA

SEQ ID NO: 59 - protein, 7 aa, human
AASVLQS

SEQ ID NO: 60 - protein, 9 aa, human
QKYDSAPYT

SEQ ID NO: 61 - protein, 5 aa, human
SHAIS

SEQ ID NO: 62 - protein, 17 aa, human
GITAVFGTANYAQKFQG

SEQ ID NO: 63 - protein, 13 aa, human
SLGYHTQYNGMDV

SEQ ID NO: 64 - protein, 11 aa, human
RASQGISSWLA

SEQ ID NO: 65 - protein, 7 aa, human
AASSLQS

SEQ ID NO: 66 - protein, 9 aa, human
QQANSFPIT

SEQ ID NO: 67 - protein, 5 aa, human
NYAIS

SEQ ID NO: 68 - protein, 17 aa, human
GITPIFGAANYAQKFQG

SEQ ID NO: 69 - protein, 12 aa, human
HPTYHYGSAMDY

SEQ ID NO: 70 - protein, 12 aa, human
GSSSNIGNNYVS

SEQ ID NO: 71 - protein, 7 aa, human
EGSNRPS

SEQ ID NO: 72 - protein, 11 aa, human
ATWDDSLSVVL

SEQ ID NO: 73 - protein, 5 aa, human
KSAIT

SEQ ID NO: 74 - protein, 17 aa, human
GIIAIFGTTNYAQKFQD

SEQ ID NO: 75 - protein, 11 aa, human
GPNYYESYLDF

SEQ ID NO: 76 - protein, 11 aa, human
RASQSISSWLA

SEQ ID NO: 77 - protein, 7 aa, human
KASSLES

SEQ ID NO: 78 - protein, 9 aa, human
QQYNSYPRT

SEQ ID NO: 79 - protein, 5 aa, human
SYAIS

SEQ ID NO: 80 - protein, 17 aa, human
GISPMFGTTRYAQKFQG

SEQ ID NO: 81 - protein, 11 aa, human
SPTYYPGALDM

SEQ ID NO: 82 - protein, 11 aa, human
RASQAIANFVS

SEQ ID NO: 83 - protein, 7 aa, human
AASTLQG

SEQ ID NO: 84 - protein, 9 aa, human
QQYNSLPFT

SEQ ID NO: 85 - protein, 5 aa, human
SNAIS

SEQ ID NO: 86 - protein, 17 aa, human
GIIGIFGTANYAQKFQG

SEQ ID NO: 87 - protein, 15 aa, human
SRGYSFGYGTDYFDY

SEQ ID NO: 88 - protein, 11 aa, human
RASQSISSWLA

SEQ ID NO: 89 - protein, 7 aa, human
EASKLQS

SEQ ID NO: 90 - protein, 9 aa, human
QHYEAYPFS

SEQ ID NO: 91 - protein, 5 aa, human
SNAFS

SEQ ID NO: 92 - protein, 17 aa, human
GIITMFRKAEYAQKFQG

SEQ ID NO: 93 - protein, 12 aa, human
HSGYHLIGYFDS

SEQ ID NO: 94 - protein, 13 aa, human
SGTTSNIGNNYVS

SEQ ID NO: 95 - protein, 7 aa, human
DNNKRPS

SEQ ID NO: 96 - protein, 11 aa, human
GTWVSSLSVWV

SEQ ID NO: 97 - protein, 5 aa, human
NYAIS

SEQ ID NO: 98 - protein, 17 aa, human
GIVPMFGTTRFAQKFQG

SEQ ID NO: 99 - protein, 11 aa, human
APLIYNWYFDL

SEQ ID NO: 100 - protein, 11 aa, human
RASQGISSWLA

SEQ ID NO: 101 - protein, 7 aa, human
GASNLQS

SEQ ID NO: 102 - protein, 9 aa, human
QQARTFPVT

SEQ ID NO: 103 - protein, 5 aa, human
NYAIS

SEQ ID NO: 104 - protein, 17 aa, human
GITPIFGPANYAQRFQG

SEQ ID NO: 105 - protein, 12 aa, human
HPTYYFGSAMEY

SEQ ID NO: 106 - protein, 11 aa, human
GGDRIGTGPVH

SEQ ID NO: 107 - protein, 7 aa, human
GSTHRPS

SEQ ID NO: 108 - protein, 11 aa, human
QVWDTSTAQPI

SEQ ID NO: 109 - protein, 5 aa, human
SHAIS

SEQ ID NO: 110 - protein, 17 aa, human
GIIAIFGTTHYAQQFQG

SEQ ID NO: 111 - protein, 11 aa, human
GPNYFESYFDN

SEQ ID NO: 112 - protein, 11 aa, human
RASQTISSYLA

SEQ ID NO: 113 - protein, 7 aa, human
GASNRAT

SEQ ID NO: 114 - protein, 10 aa, human
QQRSSWPPIT

SEQ ID NO: 115 - protein, 5 aa, human
TNAIS

SEQ ID NO: 116 - protein, 17 aa, human
GITAIFGTPNYAQKFQG

SEQ ID NO: 117 - protein, 11 aa, human
SGTYYVSYFDS

SEQ ID NO: 118 - protein, 12 aa, human
RASQSLYSSHLA

SEQ ID NO: 119 - protein, 7 aa, human
GASTRAP

SEQ ID NO: 120 - protein, 9 aa, human
QQYGSSPIT

SEQ ID NO: 121 - protein, 5 aa, human
NYAIN

SEQ ID NO: 122 - protein, 17 aa, human
GIVPIFGTTRFAQKFQG

SEQ ID NO: 123 - protein, 11 aa, human
APLIYNWYYDL

SEQ ID NO: 124 - protein, 11 aa, human
RASQGISNYLA

SEQ ID NO: 125 - protein, 7 aa, human
AASTLQS

SEQ ID NO: 126 - protein, 9 aa, human
QKYNSAPHT

SEQ ID NO: 127 - protein, 5 aa, human
SYALS

SEQ ID NO: 128 - protein, 17 aa, human
GISAIFNTATYAQNVQG

SEQ ID NO: 129 - protein, 11 aa, human
SGTYYVSFFDY

SEQ ID NO: 130 - protein, 11 aa, human
RASQSIGSLMA

SEQ ID NO: 131 - protein, 7 aa, human
RASNLET

SEQ ID NO: 132 - protein, 9 aa, human
QHFKTFSRT

SEQ ID NO: 133 - protein, 5 aa, human
SNAIS

SEQ ID NO: 134 - protein, 17 aa, human
GIVALFGTANYAQKFQG

SEQ ID NO: 135 - protein, 13 aa, human
NSGYHISGFYLDY

SEQ ID NO: 136 - protein, 11 aa, human
RASQGISNYLA

SEQ ID NO: 137 - protein, 7 aa, human
TASTLLS

SEQ ID NO: 138 - protein, 9 aa, human
QEYKSVPLT

SEQ ID NO: 139 - protein, 5 aa, human
KFAIS

SEQ ID NO: 140 - protein, 17 aa, human
GIIPMFGTTNYAQKFQG

SEQ ID NO: 141 - protein, 13 aa, human
EEGYYYGSGPLDS

SEQ ID NO: 142 - protein, 17 aa, human
KSSQTVLNRSNNKNYLA

SEQ ID NO: 143 - protein, 7 aa, human
WSSTRES

SEQ ID NO: 144 - protein, 9 aa, human
QQFYDMPVT

SEQ ID NO: 145 - protein, 5 aa, human
NYAIS

SEQ ID NO: 146 - protein, 17 aa, human
GITPIFGAANYAQKFQG

SEQ ID NO: 147 - protein, 12 aa, human
HPMYHYGSAMDY

SEQ ID NO: 148 - protein, 13 aa, human
FGSRSNVGSSSVN

SEQ ID NO: 149 - protein, 7 aa, human
RNDQRPS

SEQ ID NO: 150 - protein, 11 aa, human
AAWDDSVKSVV

SEQ ID NO: 151 - protein, 5 aa, human
SYGIS

SEQ ID NO: 152 - protein, 17 aa, human
GILAIFGTTNYAQKFQG

SEQ ID NO: 153 - protein, 11 aa, human
GSTYYSSYFDQ

SEQ ID NO: 154 - protein, 11 aa, human
RASQGIRNSLA

SEQ ID NO: 155 - protein, 7 aa, human
SATTLRS

SEQ ID NO: 156 - protein, 9 aa, human
HRYDSAPLT

SEQ ID NO: 157 - protein, 5 aa, human
SYAIS

SEQ ID NO: 158 - protein, 17 aa, human
AVIPMFGTLKYAENFQG

SEQ ID NO: 159 - protein, 15 aa, human
NYYGSGTYFNDAFDI

SEQ ID NO: 160 - protein, 14 aa, human
TGGNSNIGAGYDVN

SEQ ID NO: 161 - protein, 7 aa, human
GNNNRPS

SEQ ID NO: 162 - protein, 11 aa, human
QSYDNGLSGSV

SEQ ID NO: 163 - protein, 5 aa, human
NYAIS

SEQ ID NO: 164 - protein, 17 aa, human
GITPIFGAANYAQKFQG

SEQ ID NO: 165 - protein, 12 aa, human
HPTYYYGSPMDY

SEQ ID NO: 166 - protein, 11 aa, human
RASQSIATYLN

SEQ ID NO: 167 - protein, 7 aa, human
AASNLQS

SEQ ID NO: 168 - protein, 9 aa, human
LQSYSATLT

SEQ ID NO: 169 - protein, 5 aa, human
KFAIT

SEQ ID NO: 170 - protein, 17 aa, human
GITALFATTSYAQKFQD

SEQ ID NO: 171 - protein, 11 aa, human
GPNYYETYLDN

SEQ ID NO: 172 - protein, 11 aa, human
RASQSISKWLA

SEQ ID NO: 173 - protein, 7 aa, human
MASTLES

SEQ ID NO: 174 - protein, 9 aa, human
QQYNSYPRT

SEQ ID NO: 175 - DNA, 360 bp, human
  CDS (1)..(360)
caggtgcagctgcaggagtcgggggctgaggtgaagaagcctgggtcctcggtgaaggtctcctgcaaggcctctggagggatcttcaggagaaatgctatcagctgggtgcgacaggcccctggacaagggcttgagtggatgggagggatcatcgctatctttgggacagcaaactacgcacagaagtttcagggcagagtcacgattactgcggacgaatccacgagcacagtctacatggaactgagcagcctgagacctgaggacacggccgtgtattactgtgcgagagggccgaattactatgaaaatttctttgactactggggccagggaaccctggtcaccgtctcgagc

SEQ ID NO: 176 - protein, 120 aa, human
QVQLQESGAEVKKPGSSVKVSCKASGGIFRRNAISWVRQAPGQGLEWMGGIIAIFGTANYAQKFQGRVTITADESTSTVYMELSSLRPEDTAVYYCARGPNYYENFFDYWGQGTLVTVSS

SEQ ID NO: 177 - DNA, 321 bp, human
  CDS (1)..(321)
gacatcgtgatgacccagtctccatcctccctgtctgcttctgtgggagacagacttgacctcacttgccgggcgagtcaggacatcgccaattccttagcgtggtataaacaaagaccggggaaggcccctgaactcctggtctttggtgcttccaggttggagggtggagtcccgtctagattcagtggcactagagccgggacagatttcactctcaccatcagcagtctgcagcctgaagaccttggtacttatttctgtcagcagtatttttcgtttcctcggaccttcggccaggggaccacgctggacctcaga

SEQ ID NO: 178 - protein, 107 aa, human
DIVMTQSPSSLSASVGDRLDLTCRASQDIANSLAWYKQRPGKAPELLVFGASRLEGGVPSRFSGTRAGTDFTLTISSLQPEDLGTYFCQQYFSFPRTFGQGTTLDLR

SEQ ID NO: 179 - DNA, 372 bp, human
  CDS (1)..(372)
caggtacagctgcagcagtcaggggctgaggtgaagaagcctgggtcctcggtgaaggtgtcctgcaaggcttctggaggcatcttcagaaaaaatgctatcagctgggtgcgacaggcccctggacaagggcttgagtggatgggagggatcagcgctatctttggtacggcaaactacgcacagaagttccagggcagagtcacgattaccgcggacgaatcgtcgaatacagtctatatggacctgagcaggctgagatctgaggacacggccatttattactgtgcgagccaccctacctatcactttgataaaagtggttatcgctttgactcctggggccagggaaccctggtcaccgtctcgagc

SEQ ID NO: 180 - protein, 124 aa, human
QVQLQQSGAEVKKPGSSVKVSCKASGGIFRKNAISWVRQAPGQGLEWMGGISAIFGTANYAQKFQGRVTITADESSNTVYMDLSRLRSEDTAIYYCASHPTYHFDKSGYRFDSWGQGTLVTVSS

SEQ ID NO: 181 - DNA, 333 bp, human
  CDS (1)..(333)
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctggggtgttcggcggagggaccaagctgaccgtcctaggt

SEQ ID NO: 182 - protein, 111 aa, human
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVLG

SEQ ID NO: 183 - DNA, 378 bp, human
  CDS (1)..(378)
caggtgcagctggtgcagtctggggctgaagtgaagaagcctgggtcctcggtgaaggtctcctgcaaggcttctggaggcaccttcagtagcttcagcacctatactatcagctgggtgcgacaggcccctggacaagggcttgagtggatgggaaggtccatccctctccttggtataacaaactacgcacagaagttccagggcagagtcacgattaccgcggacatatccacgagcacagcctacatggagctgagcagcctgagatctgaggacacggccgtgtattactgtgcgagataccagagtagtgactattacaactctgaatacttccaacactggggccagggcaccctggtcaccgtctcgagc

SEQ ID NO: 184 - protein, 126 aa, human
QVQLVQSGAEVKKPGSSVKVSCKASGGTFSSFSTYTISWVRQAPGQGLEWMGRSIPLLGITNYAQKFQGRVTITADISTSTAYMELSSLRSEDTAVYYCARYQSSDYYNSEYFQHWGQGTLVTVSS

SEQ ID NO: 185 - DNA, 324 bp, human
  CDS (1)..(324)
gaaattgtgttgacgcagtctccaggcaccctgtctttgtctctaggggaaagagtcaccctctcctgcagggccagtcagaatgttggcaccaattacttagcctggtaccagcagaaacctggccaggctcccaggctcctcatctatggtgcatccagcagggccactggcgtcccagacaggttcagtggcagtgggtctgggacagacttcactctcaccatcagcagactggcgcctgaagattttgcagtgtactactgtcagcagtatagtagttcacctctcactttcggcggagggaccaaactggagatcaag

SEQ ID NO: 186 - protein, 108 aa, human
EIVLTQSPGTLSLSLGERVTLSCRASQNVGTNYLAWYQQKPGQAPRLLIYGASSRATGVPDRFSGSGSGTDFTLTISRLAPEDFAVYYCQQYSSSPLTFGGGTKLEIK

SEQ ID NO: 187 - DNA, 360 bp, human
  CDS (1)..(360)
caggtgcagctggtgcagtctggggccgaggtgaagaagcctgggtcctcggtgaaggtctcctgcaaggcctctggaggcattttccgcaaaaatgccatcagctacgtgcgccaggcccctggacaaggggttgagtggatgggagggatcaccgctatctttggcacaccaaactacgcacagaagttccagggcagaatcaccattagcgcggacgaatccacgaacacagtctacatggaactgggcagtctgacatctgaggacacggccgtttattattgtgcgaccagtgggacctactacgtctcctaccttgactcctggggccagggaaccctggtcaccgtctcgagc

SEQ ID NO: 188 - protein, 120 aa, human
QVQLVQSGAEVKKPGSSVKVSCKASGGIFRKNAISYVRQAPGQGVEWMGGITAIFGTPNYAQKFQGRITISADESTNTVYMELGSLTSEDTAVYYCATSGTYYVSYLDSWGQGTLVTVSS

SEQ ID NO: 189 - DNA, 324 bp, human
  CDS (1)..(324)
gatgttgtgatgactcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggccctgggaccaaagtggatatcaaa

SEQ ID NO: 190 - protein, 108 aa, human
DVVMTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGPGTKVDIK

SEQ ID NO: 191 - DNA, 360 bp, human
  CDS (1)..(360)
gaggtgcagctggtggagtctggggccgaggtgaagaagcctgggtcctcggtgaaggtctcctgcaaggcttctggaggccacttcaacgcctatggaatcagctgggtgcggcaggccccaggacaaggccttgagtggatgggagggattgtcgccctctttggaacaacaaactacgcacagaagttgcagggcagagtcacaattaccgcggacgcgtccacgaacacagtgtacatggagttgaccagcctgagatctgacgacacggccgtatattactgtgcgagaagcgggagttattaccctgattacttccaatattggggccagggaaccctggtcaccgtctcgagc

SEQ ID NO: 192 - protein, 120 aa, human
EVQLVESGAEVKKPGSSVKVSCKASGGHFNAYGISWVRQAPGQGLEWMGGIVALFGTTNYAQKLQGRVTITADASTNTVYMELTSLRSDDTAVYYCARSGSYYPDYFQYWGQGTLVTVSS

SEQ ID NO: 193 - DNA, 324 bp, human
  CDS (1)..(324)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatccatggtgcatccagcagggccactggcatcccagacaggttcagtggcagtgggtctgggacagacttcactctctccatcagcagactggagcctgaagattttgcagtgtattactgtcagcagtatggtagctcacctcggacgttcggccaagggaccaaggtggaaatcaaa

SEQ ID NO: 194 - protein, 108 aa, human
EIVLTQSPGTLSLSPGERATLSCRASQSVSSSYLAWYQQKPGQAPRLLIHGASSRATGIPDRFSGSGSGTDFTLSISRLEPEDFAVYYCQQYGSSPRTFGQGTKVEIK

SEQ ID NO: 195 - DNA, 366 bp, human
  CDS (1)..(366)
caggtgcagctggtgcaatctgggtctgaggtgaagaagcctgggtcctcggtgaaggtctcctgctccttcaaggcatctggaggcatcatcagcaagtatgccatcacctgggtgcgacaggcccctggacaagggcttgagtggatgggagggatcatcgctatctttggttcaacaaactacgcacagaagttccagggcagactcacgattacctcggacgaatccacgagcacagcctatatggaactgagcagcctgacatctgaagacacggccgtgtattattgtgggagaggaccacattactatgagagccaccttgactactggggccagggaaccctggtcaccgtctcgagc

SEQ ID NO: 196 - protein, 122 aa, human
QVQLVQSGSEVKKPGSSVKVSCSFKASGGIISKYAITWVRQAPGQGLEWMGGIIAIFGSTNYAQKFQGRLTITSDESTSTAYMELSSLTSEDTAVYYCGRGPHYYESHLDYWGQGTLVTVSS

SEQ ID NO: 197 - DNA, 324 bp, human
  CDS (1)..(324)
gaaattgtgttgacgcagtctccaggcaccctgtctttgtctccaggggagagagccaccctctcctgcagggccagtcagggtatgagcagcagcttcttagcctggtaccaacagaagcctggccagcctcccaggctcctcatctctggtgcatccaccagggccactggcatcccagacaggttcagtggcagtgggtctgggacagacttcactctcaccatcaacagactggagcctgaagattttgcagtgtattactgtcagcagtatggtagctcacctcggacgttcggccaagggaccaaggtggaaatcaaa

SEQ ID NO: 198 - protein, 108 aa, human
EIVLTQSPGTLSLSPGERATLSCRASQGMSSSFLAWYQQKPGQPPRLLISGASTRATGIPDRFSGSGSGTDFTLTINRLEPEDFAVYYCQQYGSSPRTFGQGTKVEIK

SEQ ID NO: 199 - DNA, 360 bp, human
  CDS (1)..(360)
caggtgcagctggtgcaatctgggtctgaggtgaagaagcctgggtcctcggtgaaggtctcctgcaagtcttctggaggcatcctcaggaggtctgccatcagctgggtgcgacaggcccctggacaagggcttgagtggatgggagggatcctcgctatctttggtacaacaaagtacgcacagaagttccagggcagaatcaccattaccgcggacgaatccacgaatacagtccacatggagctgagcagcctgagacctgacgacacggccgtgtattactgtgcgggaggcccacactattatgtttcctactttgactcctggggccagggaaccctggtcaccgtctcgagc

SEQ ID NO: 200 - protein, 120 aa, human
QVQLVQSGSEVKKPGSSVKVSCKSSGGILRRSAISWVRQAPGQGLEWMGGILAIFGTTKYAQKFQGRITITADESTNTVHMELSSLRPDDTAVYYCAGGPHYYVSYFDSWGQGTLVTVSS

SEQ ID NO: 201 - DNA, 321 bp, human
  CDS (1)..(321)
gacatccagatgacccagtctccagccaccctgtctttgtctccaggggagagagccaccctctcctgcagggccagtcagagtgttaggagctacttagcctggtaccaacagaaacctggccaggctcccaggctcctcatctatgatgcatccaacagggccactggcatcccagccaggttcagcggcagtgggtctgggacagagttcactctcaccatcagccgcctagagcctgaagattttgcagtttattactgtcagcaacgtagtaactggttattcactttcggccctgggaccaaagtggatttcaaa

SEQ ID NO: 202 - protein, 107 aa, human
DIQMTQSPATLSLSPGERATLSCRASQSVRSYLAWYQQKPGQAPRLLIYDASNRATGIPARFSGSGSGTEFTLTISRLEPEDFAVYYCQQRSNWLFTFGPGTKVDFK

SEQ ID NO: 203 - DNA, 360 bp, human
  CDS (1)..(360)
caggtgcagctggtgcagtctggggctgaggtgaagaagcctgggtcctcggtgaaggtctcctgcaaggcttctggaggcatcttcaggagcaatgctgtcagctgggtgcgacaggcccctggccaagggcttgagtggatgggagggatcatcgctatctttggtacaccaaagtatgcacagaagttccagggcagagtcaccattaccgcggacgaatccacgagcacagtttacatggagttgagcggtctgagatatgaggacacggccgtctattactgtgcgagagggccgaattactatgaaagttacttcgactattggggccagggaaccctggtcaccgtctcgagc

SEQ ID NO: 204 - protein, 120 aa, human
QVQLVQSGAEVKKPGSSVKVSCKASGGIFRSNAVSWVRQAPGQGLEWMGGIIAIFGTPKYAQKFQGRVTITADESTSTVYMELSGLRYEDTAVYYCARGPNYYESYFDYWGQGTLVTVSS

SEQ ID NO: 205 - DNA, 321 bp, human
  CDS (1)..(321)
gacatcgtgatgacccagtctccaccctccctgtctgcatctgtaggagacagagtcaccatcacttgccgggcgagtcagggcatcagtaattatttagcctggtatcagcagaaaccagggaaagctcccaagctcctgatctttggtgcatctactttgcactcaggggtctcgtctcggttcagtggcagcggatctgggccagatttcactctcaccatcagcagtctgcagcctgaagatgctgcaacttattactgtcaaaagtatcacagtgcccctctcactttcggcggagggaccaaggtggagatgaaa

SEQ ID NO: 206 - protein, 107 aa, human
DIVMTQSPPSLSASVGDRVTITCRASQGISNYLAWYQQKPGKAPKLLIFGASTLHSGVSSRFSGSGSGPDFTLTISSLQPEDAATYYCQKYHSAPLTFGGGTKVEMK

SEQ ID NO: 207 - DNA, 360 bp, human
  CDS (1)..(360)
caggtgcagctggtgcagtctggggctgaggtgaagaagcctgggtcctcggtgaaggtctcctgcaaggcttctggaggcatcttcaacaagtatggtatcagctgggtgcgacaggcccctggacaagggcttgagtggatgggggggatcctcgcaattttcggaacaacaaactacgcacagaagttccagggcagagtcacgattagcgcggacgagtcctcgactacagtgtacatggagctgagcagcctgacgtctgaggacacggccgtgtattattgtgcgagggggaatacttattactccagttactttgaccagtggggccagggcaccctggtcaccgtctcgagc

SEQ ID NO: 208 - protein, 120 aa, human
QVQLVQSGAEVKKPGSSVKVSCKASGGIFNKYGISWVRQAPGQGLEWMGGILAIFGTTNYAQKFQGRVTISADESSTTVYMELSSLTSEDTAVYYCARGNTYYSSYFDQWGQGTLVTVSS

SEQ ID NO: 209 - DNA, 330 bp, human
  CDS (1)..(330)
cagtctgtgttgacgcagccgccctcggtgtcagtggccccaggtcaaacggccaccatcacctgtgggggagacaagattgggactggacctgtgcattggtatcagcagatgccaggccgggcccctgtcctgctcatttatgccagcactcatcggccctcaaggatccctgagcggttctctggctccaagtctgacaatacggccaccctctccatcagcggggtcggagtcggggatgaggccgactattactgtcagatgtgggacagttcctctgtccatccgattgttttcggcggggggaccaagctgaccgtcctcagt

SEQ ID NO: 210 - protein, 110 aa, human
QSVLTQPPSVSVAPGQTATITCGGDKIGTGPVHWYQQMPGRAPVLLIYASTHRPSRIPERFSGSKSDNTATLSISGVGVGDEADYYCQMWDSSSVHPIVFGGGTKLTVLS

SEQ ID NO: 211 - DNA, 360 bp, human
  CDS (1)..(360)
caggtacagctgcagcagtcaggggctgaggtgaagaagcctgggtcctcggtgaaggtctcctgcaaggcttctggaggcatcttcaggaagaatgctatcagctgggtgcgacaggcccccggacaagggcttgagtggatgggagggatcatcgctatctttggtacagcaaactacgcacagaagttccagggcagagtcacgattaccgcggacgattcaacgaacacagtctacatggagctgaccagcctgagatatgaggacacggccgtgtattactgtgcgagagggccgaattactatgaaaattactttgacttctggggccagggcaccctggtcaccgtctcgagc

SEQ ID NO: 212 - protein, 120 aa, human
QVQLQQSGAEVKKPGSSVKVSCKASGGIFRKNAISWVRQAPGQGLEWMGGIIAIFGTANYAQKFQGRVTITADDSTNTVYMELTSLRYEDTAVYYCARGPNYYENYFDFWGQGTLVTVSS

SEQ ID NO: 213 - DNA, 321 bp, human
  CDS (1)..(321)
gaaattgtgttgacgcagtctccatcctccctgtctgcatctttgagagacactgtcaccatcacttgccggacgagccaggccattaccaattatttagcctggtatcaacagaggccaggtaaagctcctaaactcctgatctatgctgcatccgttttgcaatcaggggtcccatctcgtttcagtggcagtggatctgggacagatttcactctcaccatcagcagcctgcagcctgaagattttgcaacttattactgtcaaaagtatgacagtgccccgtacacttttggccaggggaccaagctggagatcaaa

SEQ ID NO: 214 - protein, 107 aa, human
EIVLTQSPSSLSASLRDTVTITCRTSQAITNYLAWYQQRPGKAPKLLIYAASVLQSGVPSRFSGSGSGTDFTLTISSLQPEDFATYYCQKYDSAPYTFGQGTKLEIK

SEQ ID NO: 215 - DNA, 366 bp, human
  CDS (1)..(366)
caggtgcagctgcaggagtcgggggctgaggtgaagaagcctgggtcctcggtgaaggtctcctgcaaggcttctggaggacccttcaggagccatgctattagctgggtgcgacaggccgctggacaagggcttgagtggatgggagggatcaccgctgtctttggtacagcaaactacgcacagaagttccagggcagagtcacgataaccgcggacgaatccacgaacatagtctccatggagctgagcagcctgagatctgaggacacggccatctattactgtgcgagaagccttggctaccacactcagtacaacggtatggacgtctggggccaagggaccacggtcaccgtctcgagc

SEQ ID NO: 216 - protein, 122 aa, human
QVQLQESGAEVKKPGSSVKVSCKASGGPFRSHAISWVRQAAGQGLEWMGGITAVFGTANYAQKFQGRVTITADESTNIVSMELSSLRSEDTAIYYCARSLGYHTQYNGMDVWGQGTTVTVSS

SEQ ID NO: 217 - DNA, 321 bp, human
  CDS (1)..(321)
gatgttgtgatgactcagtctccatcttctgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcactatcagcagcctgcagcctgaagattttgcaacttactattgtcaacaggctaacagtttcccgatcaccttcggccaagggacacgactggagattaaa

SEQ ID NO: 218 - protein, 107 aa, human
DVVMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIK

SEQ ID NO: 219 - DNA, 363 bp, human
  CDS (1)..(363)
caggtacagctgcagcagtcaggggctgacgtgaagaaggctgggtcctcggtgaaggtctcctgcaaggcttctggacgcaccttcggcaattatgctatcagctgggtgcgacaggcccctggacaaggacttgagtggatgggagggatcacccctatctttggagcagcaaactacgcacagaagttccagggcagagtcactatttccgcggacttatccacgagaatggtctatatggacttgagcagcctgagatctgacgacacggccttctattactgtgcgggacatcccacgtatcattatggttcggcgatggactactggggccagggaaccctggtcaccgtctcgagc

SEQ ID NO: 220 - protein, 121 aa, human
QVQLQQSGADVKKAGSSVKVSCKASGRTFGNYAISWVRQAPGQGLEWMGGITPIFGAANYAQKFQGRVTISADLSTRMVYMDLSSLRSDDTAFYYCAGHPTYHYGSAMDYWGQGTLVTVSS

SEQ ID NO: 221 - DNA, 333 bp, human
  CDS (1)..(333)
cagtctgtgttgacgcagccgccctcagtgtctgcggccccaggccagaaggtcaccatcacctgttctggaagcagctccaacattggcaataattatgtgtcctggtaccagcaactcccagggacagcccccaaactcctcatttctgaaggaagtaatcgaccctccgggattcctgaccgcttctctggctccaagtctggcacgtcagccaccctgggcatcaccggactccagactggggacgaggccacttattactgcgcaacatgggatgacagcctgagtgtcgtgcttttcggcggagggacccgactgaccgtccttagt

SEQ ID NO: 222 - protein, 111 aa, human
QSVLTQPPSVSAAPGQKVTITCSGSSSNIGNNYVSWYQQLPGTAPKLLISEGSNRPSGIPDRFSGSKSGTSATLGITGLQTGDEATYYCATWDDSLSVVLFGGGTRLTVLS

SEQ ID NO: 223 - DNA, 360 bp, human
  CDS (1)..(360)
caggtgcagctggtgcagtctggggctgaggtgaagaagcctgggtcctcggtgaaggtctcctgcaaggcttctggaggcatcttcaggaagagtgctatcacctgggtgcgacaggcccccggacaagggcttgagtggatgggagggatcatcgctatctttggtacaacaaactacgctcagaagttccaggacagagtcacgattaccgcggacgattccacgaacacagtctacatggaactgaccagcctgagatatgaggacacggccgtctattactgtgcgagagggcccaattactatgaaagttaccttgacttctggggccaggggaccacggtcaccgtctcgagc

SEQ ID NO: 224 - protein, 120 aa, human
QVQLVQSGAEVKKPGSSVKVSCKASGGIFRKSAITWVRQAPGQGLEWMGGIIAIFGTTNYAQKFQDRVTITADDSTNTVYMELTSLRYEDTAVYYCARGPNYYESYLDFWGQGTTVTVSS

SEQ ID NO: 225 - DNA, 321 bp, human
  CDS (1)..(321)
gaaattgtgttgacgcagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttatccccggacgttcggccaagggaccaaggtggaaatcaaa

SEQ ID NO: 226 - protein, 107 aa, human
EIVLTQSPSTLSASVGDRVTITCRASQSISSWLAWYQQKPGKAPKLLIYKASSLESGVPSRFSGSGSGTEFTLTISSLQPDDFATYYCQQYNSYPRTFGQGTKVEIK

SEQ ID NO: 227 - DNA, 360 bp, human
  CDS (1)..(360)
caggtgcagctggtgcaatctgggtctgaggtgaagaagcctgggtcctcggtgaaggtctcctgcaaggcttctggagtcaccttcagcagttatgctatcagctgggtgcgacaggcccccggacaagggcttgagtggatgggagggatcagccctatgtttggaacgacaaggtatgcacagaagttccaggggagagtcacgattaccgcggacgaatccacgaggacaggcttcatggagctgagcagcctgagatctggggacacggccgtgtattactgtgcgagatccccaacttattatcctggcgctcttgatatgtggggccaagggaccacggtcaccgtctcgagc

SEQ ID NO: 228 - protein, 120 aa, human
QVQLVQSGSEVKKPGSSVKVSCKASGVTFSSYAISWVRQAPGQGLEWMGGISPMFGTTRYAQKFQGRVTITADESTRTGFMELSSLRSGDTAVYYCARSPTYYPGALDMWGQGTTVTVSS

SEQ ID NO: 229 - DNA, 321 bp, human
  CDS (1)..(321)
gatgttgtgatgactcagtctccatctttgctgtctgcatctgtgggagacagagtcaccatcacttgtcgggcgagtcaggccatcgccaatttcgtatcctggtttcagcagaaaccaggggaagcccctaagtccctgatccatgctgcatccactttgcaaggtggagtcccaccgagattcagcggcagtggatatggggcggttttcactctcaccatcaccaacctgcagccggaagattttgcaacttatttctgccaacaatataacagtctccccttcactttcggccctgggaccgatgtggaagtgcga

SEQ ID NO: 230 - protein, 107 aa, human
DVVMTQSPSLLSASVGDRVTITCRASQAIANFVSWFQQKPGEAPKSLIHAASTLQGGVPPRFSGSGYGAVFTLTITNLQPEDFATYFCQQYNSLPFTFGPGTDVEVR

SEQ ID NO: 231 - DNA, 372 bp, human
  CDS (1)..(372)
caggtgcagctggtgcagtctggggctgaggtgaagaagcctgggtcctcggtgaaggtctcctgcaaggcttctggaggcatcttcaacagtaatgctatcagctgggtgcgacaggcccctggacaagggcttgagtggatgggagggatcatcggtatctttggaacagcaaactacgcacagaagttccagggcagagtaacgattgccgcggaccaatccacgaacacagtcttcatggagttgagtagcctgacatctgaggacacggccgtgtatttctgtgcgagatcccgtggatacagctttggttacggcaccgactactttgactactggggccagggcaccctggtcaccgtctcgagc

SEQ ID NO: 232 - protein, 124 aa, human
QVQLVQSGAEVKKPGSSVKVSCKASGGIFNSNAISWVRQAPGQGLEWMGGIIGIFGTANYAQKFQGRVTIAADQSTNTVFMELSSLTSEDTAVYFCARSRGYSFGYGTDYFDYWGQGTLVTVSS

SEQ ID NO: 233 - DNA, 321 bp, human
  CDS (1)..(321)
gacatcgtgatgacccagtctccttccaccctgtctgcatctgtaggagacagagtcaccatcacttgccgggccagtcagagtattagtagttggttggcctggtatcagcaggccccagggaaagcccctaagctcctgatctatgaggcgtcgaaattacaaagcggggtcccatcaaggttcagcgccactggatctgggacagaattcactctcaccatcgccagcctgcagcctgaggattttgcaatttattactgccaacactacgaggcttacccattcagtttcggccctgggaccaagttggatgtcaaa

SEQ ID NO: 234 - protein, 107 aa, human
DIVMTQSPSTLSASVGDRVTITCRASQSISSWLAWYQQAPGKAPKLLIYEASKLQSGVPSRFSATGSGTEFTLTIASLQPEDFAIYYCQHYEAYPFSFGPGTKLDVK

SEQ ID NO: 235 - DNA, 363 bp, human
  CDS (1)..(363)
caggtgcagctggtgcagtctggggctgaggtgaagaagcctgggtcctcggtgaaggtctcctgcgaggcctctggagccaccttcagcagtaatgctttcagctgggtgcggcaggcccctggacaagggcctgagtggatgggagggatcatcactatgtttagaaaagcagagtacgcacagaagttccagggcagagtcacgattaccgcgggcgaactggggagtacggcctacatggaggtgagaagcctgacatttgaggacacggccgtgtattactgcgcgagacatagtggttaccatttgatcggctactttgactcctggggccagggcaccctggtcaccgtctcgagc

SEQ ID NO: 236 - protein, 121 aa, human
QVQLVQSGAEVKKPGSSVKVSCEASGATFSSNAFSWVRQAPGQGPEWMGGIITMFRKAEYAQKFQGRVTITAGELGSTAYMEVRSLTFEDTAVYYCARHSGYHLIGYFDSWGQGTLVTVSS

SEQ ID NO: 237 - DNA, 333 bp, human
  CDS (1)..(333)
cagtctgtgttgacgcagccgccctcagtgtctgcggccccaggacagaaggtcaccatctcctgctctggaaccacttccaacattggaaataattatgtttcctggtaccagcaactcccaggggcagcccccaaactcctcatttatgacaataataagcgaccctcagggattcctgaccgattctctggctccaagtctgacacgtcagccaccctgggcatcaccggactccagactggggacgaggccgattattactgcggaacatgggttagcagcctgagtgtctgggtgttcggcggagggaccaagctgaccgtcctaggt

SEQ ID NO: 238 - protein, 111 aa, human
QSVLTQPPSVSAAPGQKVTISCSGTTSNIGNNYVSWYQQLPGAAPKLLIYDNNKRPSGIPDRFSGSKSDTSATLGITGLQTGDEADYYCGTWVSSLSVWVFGGGTKLTVLG

SEQ ID NO: 239 - DNA, 360 bp, human
  CDS (1)..(360)
gaggtgcagctggtggagtctggggctgaggtgaagaagcctgggtcctcggtgaaggtctcctgcaaggcttccggaagcatcttcagcaactatgccatcagctgggtgcgacaggcccctggacaggggcttgagtggatgggaggcatcgtccctatgtttggtacaacaagattcgcacagaagttccagggcagagtcacgattaccgcggacgaatccaggagcacagcctacatggagctgaacaacctgatatctgaagacacggccgtgtattactgtgcgagagctcccctcatttataactggtacttcgatctctggggccgtggaaccctggtcaccgtctcgagc

SEQ ID NO: 240 - protein, 120 aa, human
EVQLVESGAEVKKPGSSVKVSCKASGSIFSNYAISWVRQAPGQGLEWMGGIVPMFGTTRFAQKFQGRVTITADESRSTAYMELNNLISEDTAVYYCARAPLIYNWYFDLWGRGTLVTVSS

SEQ ID NO: 241 - DNA, 321 bp, human
  CDS (1)..(321)
gaaattgtgttgacgcagtctccatcttttgtgtctgcgtcggtcggagacagagtcagcatcacttgccgggcgagtcagggtattagcagctggttagcctggtatcaacaaaaacccgggaaagcccctagactcctgatctatggtgcatccaacttgcaaagtggggtcccatcaaggttcagcggcagtggatctgggacagattacactctcactatcaacagcctgcagcctgaagattttgcaacttactactgtcaacaggctcgcaccttcccggtaacttttggccaggggaccaagctggagatcaaa

SEQ ID NO: 242 - protein, 107 aa, human
EIVLTQSPSFVSASVGDRVSITCRASQGISSWLAWYQQKPGKAPRLLIYGASNLQSGVPSRFSGSGSGTDYTLTINSLQPEDFATYYCQQARTFPVTFGQGTKLEIK

SEQ ID NO: 243 - DNA, 363 bp, human
  CDS (1)..(363)
caggtacagctgcagcagtcaggggctgacgtgaagaagcctgggtcctcggtgaaggtctcctgcaaggcttctggaggcaccttcggcaactatgctatcagctgggtgcgacaggcccctggacaagggcttgagtggatgggagggatcacccctatctttggaccagcaaactacgcacagaggttccagggcagagtcacgatttccgcggacatatccacgaggacagcctacatggagctgagcagcctgagatctgaggacacggccatatattactgtgcgagacatcctacgtattattttggttcggcgatggagtactggggccagggcaccctggtcaccgtctcgagc

SEQ ID NO: 244 - protein, 121 aa, human
QVQLQQSGADVKKPGSSVKVSCKASGGTFGNYAISWVRQAPGQGLEWMGGITPIFGPANYAQRFQGRVTISADISTRTAYMELSSLRSEDTAIYYCARHPTYYFGSAMEYWGQGTLVTVSS

SEQ ID NO: 245 - DNA, 330 bp, human
  CDS (1)..(330)
tcctatgagctgactcagccaccctcggtgtcagtggcccccggtcaaacggccaccatcacctgtgggggagacaggattgggactggacctgtacattggtatcagcagaggccaggccgggcccctgtcctgatcatttatggcagcactcatcggccctcagggatccctgagcgattctctggctccaagtctgtcaatacggccaccctgtccatcagcggggtcggagtcggggatgaggccgactattactgtcaggtgtgggacacttccactgcccagccgattgttttcggcggagggaccaagctgaccgtcctcagt

SEQ ID NO: 246 - protein, 110 aa, human
SYELTQPPSVSVAPGQTATITCGGDRIGTGPVHWYQQRPGRAPVLIIYGSTHRPSGIPERFSGSKSVNTATLSISGVGVGDEADYYCQVWDTSTAQPIVFGGGTKLTVLS

SEQ ID NO: 247 - DNA, 360 bp, human
  CDS (1)..(360)
caggtgcagctggtgcagtctggggctgaggtaaagaagcctgggtcctcggtgaaggtctcctgcaaggcttctggaggcatcttcagaagtcatgctatcagctgggtgcgacaggcccctggacaagggcttgaatggatgggagggataatcgctatatttggtacaacacactacgcacagcagttccagggcagagtcacaatcaccgcggacgaatccacgagcacagtgtacatggagctgagcaccctgagatctgaggacacggccgtgtattactgtgcgagagggccgaactactttgagagttactttgacaactggggccagggaaccctggtcaccgtctcgagc

SEQ ID NO: 248 - protein, 120 aa, human
QVQLVQSGAEVKKPGSSVKVSCKASGGIFRSHAISWVRQAPGQGLEWMGGIIAIFGTTHYAQQFQGRVTITADESTSTVYMELSTLRSEDTAVYYCARGPNYFESYFDNWGQGTLVTVSS

SEQ ID NO: 249 - DNA, 324 bp, human
  CDS (1)..(324)
gaaattgtgttgacgcagtctccagccaccctgtctttgtctccaggggaaagagccaccctctcctgcagggccagtcagactattagcagctacttagcctggtaccaacagaaacctggccaggctcccaggctcctcatctatggtgcatccaacagggccactggcatcccagccaggttcagtggcagtgggtctgggacagacttcactctcaccatcaacagcctagagcctgaagattttgcagtttattactgtcagcagcgtagttcctggcctccgatcaccttcggccaagggacacgactggagattaaa

SEQ ID NO: 250 - protein, 108 aa, human
EIVLTQSPATLSLSPGERATLSCRASQTISSYLAWYQQKPGQAPRLLIYGASNRATGIPARFSGSGSGTDFTLTINSLEPEDFAVYYCQQRSSWPPITFGQGTRLEIK

SEQ ID NO: 251 - DNA, 360 bp, human
  CDS (1)..(360)
caggtgcagctggtgcaatctgggtctgaggtgaagaagcctgggtcctcggtgaaggtctcctgcaaggcttctggaggcattttccgcactaatgccatcagctacgtgcgacaggcccctggacaagggcttgagtggatgggagggatcaccgctatctttggcacaccaaactacgcacagaagttccagggcagagtcaccattatcgcggacgaatccacgagcacagtctacatggaactgggcagtctgacatctgaggacacggccgtgtattattgtgcgaccagtgggacctactacgtctcctactttgactcctggggccagggaaccctggtcaccgtctcgagc

SEQ ID NO: 252 - protein, 120 aa, human
QVQLVQSGSEVKKPGSSVKVSCKASGGIFRTNAISYVRQAPGQGLEWMGGITAIFGTPNYAQKFQGRVTIIADESTSTVYMELGSLTSEDTAVYYCATSGTYYVSYFDSWGQGTLVTVSS

SEQ ID NO: 253 - DNA, 324 bp, human
  CDS (1)..(324)
gacatcgtgatgacccagtctccaggcaccctgactttgtctccaggggaaagagccaccctctcctgcagggccagtcagagtctttatagcagccacttagcctggtaccagcagaaacctggccagcctcccaggctcctcatctatggtgcatccaccagggccccgggcactccagacaggttcagtggcagtgggtctgggacagacttcactctcaccatcaccgccctggaccctgaagattctgcagtgtattactgtcagcaatatggtagctcaccgatcaccttcggccaagggacgcgactggagattaaa

SEQ ID NO: 254 - protein, 108 aa, human
DIVMTQSPGTLTLSPGERATLSCRASQSLYSSHLAWYQQKPGQPPRLLIYGASTRAPGTPDRFSGSGSGTDFTLTITALDPEDSAVYYCQQYGSSPITFGQGTRLEIK

SEQ ID NO: 255 - DNA, 360 bp, human
  CDS (1)..(360)
gaggtgcagctggtgcagtctggggctgaggtgaagaagcctgggtcctcggtgaaggtctcctgccaggcttccggaagcatcttcagcaactatgccatcaactgggtgcgccaggcccctggacaggggcttgagtggatgggaggcatcgtccctatatttggtacaacaagattcgcacagaagttccagggcagactcacgattaccgcggacgaacccaggagcacagcctacatggagctgaacaacctgatatctgaagacacggccgtgtattactgtgcgagagctcccctcatttataactggtactacgatctctggggccgtggaaccctggtcaccgtctcgagc

SEQ ID NO: 256 - protein, 120 aa, human
EVQLVQSGAEVKKPGSSVKVSCQASGSIFSNYAINWVRQAPGQGLEWMGGIVPIFGTTRFAQKFQGRLTITADEPRSTAYMELNNLISEDTAVYYCARAPLIYNWYYDLWGRGTLVTVSS

SEQ ID NO: 257 - DNA, 321 bp, human
  CDS (1)..(321)
gaaattgtgttgacgcagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgcccctcacactttcggccctgggaccaaagtggatatcaaa

SEQ ID NO: 258 - protein, 107 aa, human
EIVLTQSPSSLSASVGDRVTITCRASQGISNYLAWYQQKPGKVPKLLIYAASTLQSGVPSRFSGSGSGTDFTLTISSLQPEDVATYYCQKYNSAPHTFGPGTKVDIK

SEQ ID NO: 259 - DNA, 360 bp, human
  CDS (1)..(360)
caggtgcagctggtgcagtctggggctgaggtgaagaagcctgggtcctcggtgaagctctcctgcaaggcttctggcggcaccctccgaagttatgctctcagctgggtgcgacaggcccctggagaagggcttgagtggatgggagggatcagcgccatctttaatactgcaacttacgcacagaacgtccagggcagagtcacgatcaccgcggacgagtccacgaacacattttatttggaggtgagcagcctgagatctgacgacacggccgtctattactgtgcgacatccgggacctactacgtctcattttttgactactggggccagggaaccctggtcaccgtctcgagc

SEQ ID NO: 260 - protein, 120 aa, human
QVQLVQSGAEVKKPGSSVKLSCKASGGTLRSYALSWVRQAPGEGLEWMGGISAIFNTATYAQNVQGRVTITADESTNTFYLEVSSLRSDDTAVYYCATSGTYYVSFFDYWGQGTLVTVSS

SEQ ID NO: 261 - DNA, 321 bp, human
  CDS (1)..(321)
gatattgtgttgacgcagtctccttccaccctgtctgcatctgtgggagacagagtcaccctcacttgccgggccagtcagagcattggtagcttgatggcctggtatcagcagcaaccagggaaaccccctaaactcctgatctatagggcgtctaatttggaaactggtgtcccatcaaggttcaccggcagtggatctgggacagaattcgctctcacaatcagcagcctgcagcctgatgatgttggaacctattactgccaacactttaagactttttcccggacgttcggccaagggaccaaggtggaaatcaaa

SEQ ID NO: 262 - protein, 107 aa, human
DIVLTQSPSTLSASVGDRVTLTCRASQSIGSLMAWYQQQPGKPPKLLIYRASNLETGVPSRFTGSGSGTEFALTISSLQPDDVGTYYCQHFKTFSRTFGQGTKVEIK

SEQ ID NO: 263 - DNA, 366 bp, human
  CDS (1)..(366)
caggtacagctgcagcagtcaggggctgaggtgaagcagcctgggtcctcggtgaaggtctcctgcaaggcttctggaggcatcttcaggagcaacgctatcagctgggtgcgacaggcccctggacaagggcttgagtggatgggaggcatcgtcgctttgtttggtacagcaaactacgcacagaagttccagggcagagtcacgattaccgcggacgaatctacgagtacagtctacatggaactgagcagcctgagatctgaggacacggcccgctattactgtgcgagaaatagtggctaccacatctctgggttctatcttgactactggggccagggaaccctggtcaccgtctcgagc

SEQ ID NO: 264 - protein, 122 aa, human
QVQLQQSGAEVKQPGSSVKVSCKASGGIFRSNAISWVRQAPGQGLEWMGGIVALFGTANYAQKFQGRVTITADESTSTVYMELSSLRSEDTARYYCARNSGYHISGFYLDYWGQGTLVTVSS

SEQ ID NO: 265 - DNA, 321 bp, human
  CDS (1)..(321)
gaaattgtgttgacgcagtctccatcctccctgtctgcatctgtaggagacagagtcaccatcacttgccgggcgagtcagggcattagcaattatttagcctggtatcagcagaggccagggaaagttcctaaactcctgatctatactgcttccactttgctatcaggggtcccatctcggttcagtggcggtggatctgggacagattacactctcaccatcagcagcctgcagcctgaagatgttgcaacttattactgtcaagagtataaaagtgtccctctcactttcggccctgggaccaaagtggagatcaaa

SEQ ID NO: 266 - protein, 107 aa, human
EIVLTQSPSSLSASVGDRVTITCRASQGISNYLAWYQQRPGKVPKLLIYTASTLLSGVPSRFSGGGSGTDYTLTISSLQPEDVATYYCQEYKSVPLTFGPGTKVEIK

SEQ ID NO: 267 - DNA, 366 bp, human
  CDS (1)..(366)
caggtgcagctggtgcagtctggggctgaggtgaggaagcctgggtcctcggtgaaggtctcctgcaaggcgtctggagtcatcttcatcaagtttgctattagctgggtgcgacaggcccctggacaagggcttgagtggatgggggggatcatccctatgtttggtacaacaaactacgcacagaagttccagggcagagtcacgattaccgcggacggatccaccaacacagtctacatggagttaagcagcctgacatctgaggacacggccgtgtattactgtgcgaaagaggagggatattactatggttcggggccgcttgactcctggggccagggaaccctggtcaccgtctcgagc

SEQ ID NO: 268 - protein, 122 aa, human
QVQLVQSGAEVRKPGSSVKVSCKASGVIFIKFAISWVRQAPGQGLEWMGGIIPMFGTTNYAQKFQGRVTITADGSTNTVYMELSSLTSEDTAVYYCAKEEGYYYGSGPLDSWGQGTLVTVSS

SEQ ID NO: 269 - DNA, 339 bp, human
  CDS (1)..(339)
gacatcgtgatgacccagtctccagacttcctgtctgtgtctctgggcgagagggtcaccatcaactgcaagtccagtcagactgttttaaacaggtccaataataagaattaccttgcctggtaccagaagaagccagggcagcctcctaggttgctcctttactggtcttctacccgggaatccggggtccctgatcgtttcagtggcagcgggtctgagactgatttcgctctcaccatcaccaacctgcaggctgaagatttggcagtttacttctgtcagcagttttatgatatgcctgtcactttcggcggagggaccaagctggagatcaaa

SEQ ID NO: 270 - protein, 113 aa, human
DIVMTQSPDFLSVSLGERVTINCKSSQTVLNRSNNKNYLAWYQKKPGQPPRLLLYWSSTRESGVPDRFSGSGSETDFALTITNLQAEDLAVYFCQQFYDMPVTFGGGTKLEIK

SEQ ID NO: 271 - DNA, 363 bp, human
  CDS (1)..(363)
caggtgcagctggtgcagtctggggctgacgtgaagaaggctgggtcctcggtgaaggtctcctgcaaggcttccggacgcaccttcggcaattatgctatcagctgggtgcgacaggcccctggacaaggacttgagtggatgggagggatcacccctatatttggagcagcaaactacgcacagaagttccagggcagagtcactatttccgcggacttatccacgagaatggtctatatggacttgagcagcctgagatctgacgacacggccttctattactgtgcgggacatcccatgtatcactatggttcggcgatggactactggggccagggcaccctggtcaccgtctcgagc

SEQ ID NO: 272 - protein, 121 aa, human
QVQLVQSGADVKKAGSSVKVSCKASGRTFGNYAISWVRQAPGQGLEWMGGITPIFGAANYAQKFQGRVTISADLSTRMVYMDLSSLRSDDTAFYYCAGHPMYHYGSAMDYWGQGTLVTVSS

SEQ ID NO: 273 - DNA, 333 bp, human
  CDS (1)..(333)
cagtctgtgttgacgcagccgccctcagcgtctgggacccccggacagagggtcagcatctcttgttttggaagcagatccaatgtcggaagcagttctgtaaactggtaccagcagctcccaggaagggcccccaaactcctcattcaaagaaatgatcagcggccctcaggggtccctgaccgattctctggctccaagtctggcacgtcagcctccctggccatcagtgggctccagtctgacgatgagtctacttattattgtgcagcctgggatgacagtgtgaagtccgtggtgttcggcggagggacgcagctgaccgtcctaggt

SEQ ID NO: 274 - protein, 111 aa, human
QSVLTQPPSASGTPGQRVSISCFGSRSNVGSSSVNWYQQLPGRAPKLLIQRNDQRPSGVPDRFSGSKSGTSASLAISGLQSDDESTYYCAAWDDSVKSVVFGGGTQLTVLG

SEQ ID NO: 275 - DNA, 360 bp, human
  CDS (1)..(360)
caggtgcagctggtgcagtctgggtctgaggtgaagaagcctgggtcctcggtgaaggtctcctgcaaggcttctggaggcatcttcaacagctatggtatcagctgggtgcgacaggcccctggacaagggcttgagtggatgggggggatcctcgctatctttggaacaacaaactacgcacagaagttccagggcagagtcacgattaccgcggacgactcctcgaaaacagtgtacatggagctgagcagcctgacatctgaggacacggccgtgtattactgtgcgagggggagtacttattactccagttactttgaccagtggggccagggaaccctggtcaccgtctcgagc

SEQ ID NO: 276 - protein, 120 aa, human
QVQLVQSGSEVKKPGSSVKVSCKASGGIFNSYGISWVRQAPGQGLEWMGGILAIFGTTNYAQKFQGRVTITADDSSKTVYMELSSLTSEDTAVYYCARGSTYYSSYFDQWGQGTLVTVSS

SEQ ID NO: 277 - DNA, 321 bp, human
  CDS (1)..(321)
gacatccagatgacccagtctccatcctccctgtctgcatctgtaggagacagagtcaccatcacttgccgggcgagtcagggcatccgcaattctttggcctggtatcagcagaaaccagggcaagttcctaagctcctaatctattctgcaaccactttgcgatcaggggtcccataccgcttcagtggcagtgggtctgggacagatttcactctcaccatcagcggcctgcagcctgaagatgttggaacttatttctgtcacaggtatgacagtgcccccctcacattcggcggagggaccagggtggcgctcaag

SEQ ID NO: 278 - protein, 107 aa, human
DIQMTQSPSSLSASVGDRVTITCRASQGIRNSLAWYQQKPGQVPKLLIYSATTLRSGVPYRFSGSGSGTDFTLTISGLQPEDVGTYFCHRYDSAPLTFGGGTRVALK

SEQ ID NO: 279 - DNA, 372 bp, human
  CDS (1)..(372)
caggtgcagctggtgcaatctgggtctgaggtgaagaagcctgggtcctcggtgaaggtctcctgcaaggcttctagaggcatcttcagcagttatgctatcagctgggtgcgacaggcccctggacaagggcttgagtggatgggagcggtcatccctatgtttggcacgctaaagtatgcagagaatttccagggcagaatcacgatatccgcggacaaatccatgagcgcagcctacatggagctgagcagcctgagatctgaggacacggccgtatattactgtgcgagaaattactatggttcggggacttatttcaatgatgcttttgatatttggggccaagggacaatggtcaccgtctcgagc

SEQ ID NO: 280 - protein, 124 aa, human
QVQLVQSGSEVKKPGSSVKVSCKASRGIFSSYAISWVRQAPGQGLEWMGAVIPMFGTLKYAENFQGRITISADKSMSAAYMELSSLRSEDTAVYYCARNYYGSGTYFNDAFDIWGQGTMVTVSS

SEQ ID NO: 281 - DNA, 336 bp, human
  CDS (1)..(336)
cagtctgtgttgacgcagccgccctcagggtctggggccccagggcagagggtcaccatctcctgcactgggggcaactccaacatcggggcaggttatgatgtaaatgggtaccagcaacttccaggaaaagcccctaaactcctcatttatggaaacaacaatcggccctcaggggtccctgaccgattctctgggtccaagtcaggcacctcagcctccctggccatcactgggctccaggttgaggatgaggctcactattactgccagtcatatgacaatggcctgagtggctcggtttttggcggagggacccagctgaccgtcctaggt

SEQ ID NO: 282 - protein, 112 aa, human
QSVLTQPPSGSGAPGQRVTISCTGGNSNIGAGYDVNGYQQLPGKAPKLLIYGNNNRPSGVPDRFSGSKSGTSASLAITGLQVEDEAHYYCQSYDNGLSGSVFGGGTQLTVLG

SEQ ID NO: 283 - DNA, 363 bp, human
  CDS (1)..(363)
caggtgcagctggtgcagtctggggctgacgtgaagaagcctgggtcctcggtgagggtctcctgcaaggcttctggacgcaccttcggcaattatgctatcagctgggtgcgacaggcccctggacaaggacttgagtggatgggagggatcacccctatctttggagcagcaaactacgcacagaagttccagggcagagtcactatttccgcggacttatccacgactatggtctacatggacttgagcagcctgagatctgacgacacggccttctattactgtgcgggacatccaacgtattactatggttcgccgatggactactggggccagggaaccctggtcaccgtctcgagc

SEQ ID NO: 284 - protein, 121 aa, human
QVQLVQSGADVKKPGSSVRVSCKASGRTFGNYAISWVRQAPGQGLEWMGGITPIFGAANYAQKFQGRVTISADLSTTMVYMDLSSLRSDDTAFYYCAGHPTYYYGSPMDYWGQGTLVTVSS

SEQ ID NO: 285 - DNA, 321 bp, human
  CDS (1)..(321)
gaaattgtgttgacgcagtctccatcctccctgtctgcatctgtaggagacagagtcaccatcacttgccgggcaagtcagagtattgccacctatttaaattggtatcagcagaaaccaggcaaggcccctaacctcctgatctatgctgcatccaatttgcaaagtggggtcccatcccggttcagtggcagtggatctgggacagatttcactctcaccatcagcagtctgcagcctgaagattttgcgacttacgtctgtctacagagttacagtgccactctcactttcggcggagggaccaaggtggagatcaaa

SEQ ID NO: 286 - protein, 107 aa, human
EIVLTQSPSSLSASVGDRVTITCRASQSIATYLNWYQQKPGKAPNLLIYAASNLQSGVPSRFSGSGSGTDFTLTISSLQPEDFATYVCLQSYSATLTFGGGTKVEIK

SEQ ID NO: 287 - DNA, 360 bp, human
  CDS (1)..(360)
caggtgcagctggtgcagtctggggctgaggtgaagaagcctgggtcctcggtgaaggtctcctgcaaggcttccggagacatattcaacaagtttgctattacttgggtgcgacaggcccctggacaagggcttgagtggatgggagggatcaccgccctctttgccacaacaagctacgcacagaagttccaggacagagtcacgattaattgggacgaatctacgaccactgccttcatggagctgagcagcctgagatttgaggacacggccgtctattactgtgcgagaggaccaaattactatgagacctaccttgacaactggggccagggcaccctggtcaccgtctcgagc

SEQ ID NO: 288 - protein, 120 aa, human
QVQLVQSGAEVKKPGSSVKVSCKASGDIFNKFAITWVRQAPGQGLEWMGGITALFATTSYAQKFQDRVTINWDESTTTAFMELSSLRFEDTAVYYCARGPNYYETYLDNWGQGTLVTVSS

SEQ ID NO: 289 - DNA, 327 bp, human
  CDS (1)..(327)
gaaattgtgttgacgcagtctcctaccatcttgtctgcgtctgtaggagacagagtcaccatcacttgccgggccagtcagagtattagtaagtggttggcctggtatcaacaaaaaccagggaaagtccctaaactcctgatctatatggcgtctaccttagaaagtggggtcccgtcgaggttcagcggcagtggatctgggtcagagttcactctcaccatcagcagcctgcagcctggtgattttgcaacttattactgccaacagtataatagttatcctcggacgttcggccaagggaccaaggtggagatcaacgtcaaa

SEQ ID NO: 290 - protein, 109 aa, human
EIVLTQSPTILSASVGDRVTITCRASQSISKWLAWYQQKPGKVPKLLIYMASTLESGVPSRFSGSGSGSEFTLTISSLQPGDFATYYCQQYNSYPRTFGQGTKVEINVK

SEQ ID NO: 291 - protein, 15 aa, Artificial Sequence
  Description of Artificial Sequence Linker
GGGGSGGGGSGGGGS

(19) United States
(12) Patent Application Publication
(10) Pub. No.: US 2016/0200799 A1
(43) Pub. Date: Jul. 14, 2016

(54) ANTI-INFLUENZA VIRUS NEUTRALIZING ANTIBODY

(71) Applicants: FUJITA HEALTH UNIVERSITY, Aichi (JP); THE RESEARCH FOUNDATION FOR MICROBIAL DISEASES OF OSAKA UNIVERSITY, Osaka (JP); PERSEUS PROTEOMICS INC., Tokyo (JP)

(72) Inventors: Yoshikazu KUROSAWA, Toyoake-shi (JP); Nobuko OSHIMA, Toyoake-shi (JP); Yoshinobu OKUNO, Kanonji-shi (JP); Katsuyuki MITOMO, Tokyo (JP); Katsushi KOUDA, Tokyo (JP)

(73) Assignees: FUJITA HEALTH UNIVERSITY, Toyoake-shi, Aichi (JP); THE RESEARCH FOUNDATION FOR MICROBIAL DISEASES OF OSAKA UNIVERSITY, Suita-shi, Osaka (JP); PERSEUS PROTEOMICS INC., Tokyo (JP)

(21) Appl. No.: 14/913,613

(22) PCT Filed: Aug. 20, 2014
(86) PCT No.: PCT/JP2014/071812

§ 371 Date: Feb. 22, 2016

(30) Foreign Application Priority Data
Aug. 23, 2013 (JP) 2013-172837

(51) Int. Cl. C07K 16/10

(52) U.S. Cl. CPC C07K 16/1018; C07K 2317/76

(57) ABSTRACT

It is an object of the present invention to provide a novel antibody having a high binding activity and a high neutralizing activity on influenza viruses. The present invention provides an antibody, which neutralizes H1 influenza virus and/or H5 influenza virus, wherein the antibody has a heavy chain variable region having CDRs consisting of a defined heavy chain first complementarity-determining region (VH CDR1), a defined heavy chain second complementarity-determining region (VH CDR2) and a defined heavy chain third complementarity-determining region (VH CDR3), and a light chain variable region having CDRs consisting of a defined light chain second complementarity-determining region (VL CDR2) and a defined light chain third complementarity-determining region (VL CDR3).

### TECHNICAL FIELD

The present invention relates to an anti-influenza virus antibody that exhibits a neutralizing activity on influenza virus.

### BACKGROUND ART

Influenza virus is an RNA enveloped virus that belongs to Orthomyxoviridae and has a particle size of approximately 100 nm in diameter, and it is divided into types A, B and C, based on the antigenicity of the internal protein thereof. The influenza virus consists of a ribonucleic acid (RNA) core associated with an internal nucleocapsid or nuclear protein, surrounded by a viral envelope, having a double-layered lipid structure, and an external glycoprotein. The inner layer of the viral envelope is mainly composed of a matrix protein, and a majority part of the outer layer is composed of a host-derived lipid substance. The RNA of the influenza virus has a segmental structure. Influenza, which spreads over the world, is caused by influenza A virus. The type A influenza virus has two types of envelope glycoproteins, hemagglutinin (HA) and neuraminidase (NA). Depending on a difference in the antigenicity, HA is divided into 16 subtypes, and NA is divided into 9 subtypes.

Recently, highly pathogenic avian influenza virus H5N1 has been prevalent over the world, and under the current circumstances, it will not be surprised even if anew virus that transmits from human to human will appear and will cause a pandemic. As measures for such situation, an international virus inspection system has been established, and a large stock of therapeutic agents such as Tamiflu and the development, production and stock of vaccines have been promoted. However, while the measures have been taken against such a new virus, many questions have remained ambiguous (e.g., When and in what a form a new virus will appear? At that time, does Tamiflu or the like exhibit therapeutic effects? Is the developed vaccine effective on the new virus? When and to whom the vaccine is given? When a state of high alert will be initiated and terminated? etc.). This is because we are facing the first situation in human history, namely, preparation of vaccines and therapeutic agents against pathogens or viruses that have not yet appeared. In particular, as a problem on the development of vaccines, with regard to the influenza virus genome, many mutations have been introduced into a hemagglutinin gene every year, so that it has caused an antigenic drift (a change in the antigenicity), and this antigenic drift is considered to cause an epidemic spread of influenza. Accordingly, if a vaccine that is incompatible with the type of epidemic influenza were given, the preventive effects could not be anticipated. The reason why the development of antibody therapeutics (preventive agents) has not been conventionally attempted is that when a therapeutic agent has been developed, the developed agent would probably become useless because the properties of a virus that could have been neutralized by the developed agent have already changed due to the antigenic drift.

The present inventors have screened a phage display human antibody library produced from a large amount of B lymphocytes collected from a single subject, against twelve influenza virus H3N2 strains, which had been separated from 1968 to 2004. As a result, the inventors have found that a majority of clones exhibiting a neutralizing activity are anti-hemagglutinin antibodies, and that these anti-hemagglutinin antibodies are broadly divided into three groups, namely, antibodies specifically neutralizing the virus strains separated from 1968 to 1973, antibodies specifically neutralizing the virus strains separated from 1977 to 1993, and antibodies specifically neutralizing the virus strains separated from 1997 to 2003 (Non Patent Literature 1). These findings defy the common technical knowledge that the development of antibody therapeutics (preventive agents) against influenza virus is senseless. However, to date, such findings have not been studied vigorously for such a reason that the combination of a heavy chain with a light chain in a phage antibody library does not necessarily reflect the situation in a living organism.

Under such circumstances, three study groups have been succeeded, by themselves, in isolation of human monoclonal antibodies that neutralize H5 influenza virus (Patent Literatures 1 and 2, and Non Patent Literature 2 to 4). It have been demonstrated that these antibodies exhibit a neutralizing activity, not only on the type H5 influenza virus, but also on influenza viruses of other subtypes (e.g., type H1). However, 16 subtypes (H1 to H16) of hemagglutinin are broadly classified into two groups (Groups 1 and 2), depending on a difference in the antigenicity.

Moreover, an isolated antibody, which neutralizes at least one influenza virus selected from Group 1 consisting of influenza viruses of subtypes H1, H2, H5, H6, H8, H9, H11, H12, H13 and H16, and at least one influenza virus selected from Group 2 consisting of influenza viruses of subtypes H3, H4, H7, H10, H14 and H15, has been reported (Patent Literature 3).

### PRIOR ART LITERATURES

### Patent Literatures

Patent Literature 1: WO 2007/134327
Patent Literature 2: WO 2008/028946
Patent Literature 3: WO 2012/029997

### Non Patent Literatures

Non Patent Literature 1: Virology, Vol. 397, pp. 322-330, 2010
Non Patent Literature 2: Proc. Natl. Acad. Sci. USA., Vol. 105, pp. 5986-5991, 2008
Non Patent Literature 3: PLOS ONE, Vol. 3, PP. 5986-5991, e3942, 2008
Non Patent Literature 4: Nature Structural & Molecular Biology, Vol. 16, pp. 265-273, 2009

### SUMMARY OF INVENTION

### Object to be Solved by the Invention

In preparation for the coming pandemic of H5N1 influenza and the expected future pandemic of H7 and H9 viruses, it has been strongly desired to develop a preventive measure that is based on a novel concept which is more universal and reliable and is substituted for the conventional preventive concept against influenza viruses wherein a change in the antigenicity is predicted and a vaccine is designed. It is an object of the present invention to provide a novel antibody having a high binding activity and a high neutralizing activity on influenza viruses.

### Means for Solving the Object

The present inventors have conducted intensive studies directed towards achieving the aforementioned object. As a result, the inventors have succeeded in obtaining a novel antibody capable of neutralizing H1 influenza virus, thereby completing the present invention.

Specifically, the present invention provides the following inventions.

[1] An antibody, which neutralizes H1 influenza virus and/or H5 influenza virus, wherein the antibody has a heavy chain variable region having CDRs consisting of a heavy chain first complementarity-determining region (VH CDR1), a heavy chain second complementarity-determining region (VH CDR2) and a heavy chain third complementarity-determining region (VH CDR3), which are described in any one of the following (1) to (29), and a light chain variable region having CDRs consisting of a light chain first complementarity-determining region (VL CDR1), a light chain second complementarity-determining region (VL CDR2) and a light chain third complementarity-determining region (VL CDR3), which are described in any one of the following (1) to (29):

### (1) VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5, and VL CDR3 of SEQ ID NO: 6,

### (2) VH CDR1 of SEQ ID NO: 7, VH CDR2 of SEQ ID NO: 8, VH CDR3 of SEQ ID NO: 9, VL CDR1 of SEQ ID NO: 10, VL CDR2 of SEQ ID NO: 11, and VL CDR3 of SEQ ID NO: 12,

### (3) VH CDR1 of SEQ ID NO: 13, VH CDR2 of SEQ ID NO: 14, VH CDR3 of SEQ ID NO: 15, VL CDR1 of SEQ ID NO: 16, VL CDR2 of SEQ ID NO: 17, and VL CDR3 of SEQ ID NO: 18,

### (4) VH CDR1 of SEQ ID NO: 19, VH CDR2 of SEQ ID NO: 20, VH CDR3 of SEQ ID NO: 21, VL CDR1 of SEQ ID NO: 22, VL CDR2 of SEQ ID NO: 23, and VL CDR3 of SEQ ID NO: 24,

### (5) VH CDR1 of SEQ ID NO: 25, VH CDR2 of SEQ ID NO: 26, VH CDR3 of SEQ ID NO: 27, VL CDR1 of SEQ ID NO: 28, VL CDR2 of SEQ ID NO: 29, and VL CDR3 of SEQ ID NO: 30,

### (6) VH CDR1 of SEQ ID NO: 31, VH CDR2 of SEQ ID NO: 32, VH CDR3 of SEQ ID NO: 33, VL CDR1 of SEQ ID NO: 34, VL CDR2 of SEQ ID NO: 35, and VL CDR3 of SEQ ID NO: 36,

### (7) VH CDR1 of SEQ ID NO: 37, VH CDR2 of SEQ ID NO: 38, VH CDR3 of SEQ ID NO: 39, VL CDR1 of SEQ ID NO: 40, VL CDR2 of SEQ ID NO: 41, and VL CDR3 of SEQ ID NO: 42,

### (8) VH CDR1 of SEQ ID NO: 43, VH CDR2 of SEQ ID NO: 44, VH CDR3 of SEQ ID NO: 45, VL CDR1 of SEQ ID NO: 46, VL CDR2 of SEQ ID NO: 47, and VL CDR3 of SEQ ID NO: 48,

### (9) VH CDR1 of SEQ ID NO: 49, VH CDR2 of SEQ ID NO: 50, VH CDR3 of SEQ ID NO: 51, VL CDR1 of SEQ ID NO: 52, VL CDR2 of SEQ ID NO: 53, and VL CDR3 of SEQ ID NO: 54,

### (10) VH CDR1 of SEQ ID NO: 55, VH CDR2 of SEQ ID NO: 56, VH CDR3 of SEQ ID NO: 57, VL CDR1 of SEQ ID NO: 58, VL CDR2 of SEQ ID NO: 59, and VL CDR3 of SEQ ID NO: 60,

### (11) VH CDR1 of SEQ ID NO: 61, VH CDR2 of SEQ ID NO: 62, VH CDR3 of SEQ ID NO: 63, VL CDR1 of SEQ ID NO: 64, VL CDR2 of SEQ ID NO: 65, and VL CDR3 of SEQ ID NO: 66,

### (12) VH CDR1 of SEQ ID NO: 67, VH CDR2 of SEQ ID NO: 68, VH CDR3 of SEQ ID NO: 69, VL CDR1 of SEQ ID NO: 70, VL CDR2 of SEQ ID NO: 71, and VL CDR3 of SEQ ID NO: 72,

### (13) VH CDR1 of SEQ ID NO: 73, VH CDR2 of SEQ ID NO: 74, VH CDR3 of SEQ ID NO: 75, VL CDR1 of SEQ ID NO: 76, VL CDR2 of SEQ ID NO: 77, and VL CDR3 of SEQ ID NO: 78,

### (14) VH CDR1 of SEQ ID NO: 79, VH CDR2 of SEQ ID NO: 80, VH CDR3 of SEQ ID NO: 81, VL CDR1 of SEQ ID NO: 82, VL CDR2 of SEQ ID NO: 83, and VL CDR3 of SEQ ID NO: 84,

### (15) VH CDR1 of SEQ ID NO: 85, VH CDR2 of SEQ ID NO: 86, VH CDR3 of SEQ ID NO: 87, VL CDR1 of SEQ ID NO: 88, VL CDR2 of SEQ ID NO: 89, and VL CDR3 of SEQ ID NO: 90,

### (16) VH CDR1 of SEQ ID NO: 91, VH CDR2 of SEQ ID NO: 92, VH CDR3 of SEQ ID NO: 93, VL CDR1 of SEQ ID NO: 94, VL CDR2 of SEQ ID NO: 95, and VL CDR3 of SEQ ID NO: 96,

### (17) VH CDR1 of SEQ ID NO: 97, VH CDR2 of SEQ ID NO: 98, VH CDR3 of SEQ ID NO: 99, VL CDR1 of SEQ ID NO: 100, VL CDR2 of SEQ ID NO: 101, and VL CDR3 of SEQ ID NO: 102,

### (18) VH CDR1 of SEQ ID NO: 103, VH CDR2 of SEQ ID NO: 104, VH CDR3 of SEQ ID NO: 105, VL CDR1 of SEQ ID NO: 106, VL CDR2 of SEQ ID NO: 107, and VL CDR3 of SEQ ID NO: 108,

### (19) VH CDR1 of SEQ ID NO: 109, VH CDR2 of SEQ ID NO: 110, VH CDR3 of SEQ ID NO: 111, VL CDR1 of SEQ ID NO: 112, VL CDR2 of SEQ ID NO: 113, and VL CDR3 of SEQ ID NO: 114,

### (20) VH CDR1 of SEQ ID NO: 115, VH CDR2 of SEQ ID NO: 116, VH CDR3 of SEQ ID NO: 117, VL CDR1 of SEQ ID NO: 118, VL CDR2 of SEQ ID NO: 119, and VL CDR3 of SEQ ID NO: 120,

### (21) VH CDR1 of SEQ ID NO: 121, VH CDR2 of SEQ ID NO: 122, VH CDR3 of SEQ ID NO: 123, VL CDR1 of SEQ ID NO: 124, VL CDR2 of SEQ ID NO: 125, and VL CDR3 of SEQ ID NO: 126,

### (22) VH CDR1 of SEQ ID NO: 127, VH CDR2 of SEQ ID NO: 128, VH CDR3 of SEQ ID NO: 129, VL CDR1 of SEQ ID NO: 130, VL CDR2 of SEQ ID NO: 131, and VL CDR3 of SEQ ID NO: 132,

### (23) VH CDR1 of SEQ ID NO: 133, VH CDR2 of SEQ ID NO: 134, VH CDR3 of SEQ ID NO: 135, VL CDR1 of SEQ ID NO: 136, VL CDR2 of SEQ ID NO: 137, and VL CDR3 of SEQ ID NO: 138,

### (24) VH CDR1 of SEQ ID NO: 139, VH CDR2 of SEQ ID NO: 140, VH CDR3 of SEQ ID NO: 141, VL CDR1 of SEQ ID NO: 142, VL CDR2 of SEQ ID NO: 143, and VL CDR3 of SEQ ID NO: 144,

### (25) VH CDR1 of SEQ ID NO: 145, VH CDR2 of SEQ ID NO: 146, VH CDR3 of SEQ ID NO: 147, VL CDR1 of SEQ ID NO: 148, VL CDR2 of SEQ ID NO: 149, and VL CDR3 of SEQ ID NO: 150,

### (26) VH CDR1 of SEQ ID NO: 151, VH CDR2 of SEQ ID NO: 152, VH CDR3 of SEQ ID NO: 153, VL CDR1 of SEQ ID NO: 154, VL CDR2 of SEQ ID NO: 155, and VL CDR3 of SEQ ID NO: 156,

### (27) VH CDR1 of SEQ ID NO: 157, VH CDR2 of SEQ ID NO: 158, VH CDR3 of SEQ ID NO: 159, VL CDR1 of SEQ ID NO: 160, VL CDR2 of SEQ ID NO: 161, and VL CDR3 of SEQ ID NO: 162,

### (28) VH CDR1 of SEQ ID NO: 163, VH CDR2 of SEQ ID NO: 164, VH CDR3 of SEQ ID NO: 165, VL CDR1 of SEQ ID NO: 166, VL CDR2 of SEQ ID NO: 167, and VL CDR3 of SEQ ID NO: 168, and

### (29) VH CDR1 of SEQ ID NO: 169, VH CDR2 of SEQ ID NO: 170, VH CDR3 of SEQ ID NO: 171, VL CDR1 of SEQ ID NO: 172, VL CDR2 of SEQ ID NO: 173, and VL CDR3 of SEQ ID NO: 174.

[2] The antibody according to [1], wherein the heavy chain variable region and the light chain variable region, respectively, consist of amino acid sequences having the amino acid sequence numbers described in any one of the following (1) to (29):

### (1) SEQ ID NO: 176 and SEQ ID NO: 178,

### (2) SEQ ID NO: 180 and SEQ ID NO: 182,

### (3) SEQ ID NO: 184 and SEQ ID NO: 186,

### (4) SEQ ID NO: 188 and SEQ ID NO: 190,

### (5) SEQ ID NO: 192 and SEQ ID NO: 194,

### (6) SEQ ID NO: 196 and SEQ ID NO: 198,

### (7) SEQ ID NO: 200 and SEQ ID NO: 202,

### (8) SEQ ID NO: 204 and SEQ ID NO: 206,

### (9) SEQ ID NO: 208 and SEQ ID NO: 210,

### (10) SEQ ID NO: 212 and SEQ ID NO: 214,

### (11) SEQ ID NO: 216 and SEQ ID NO: 218,

### (12) SEQ ID NO: 220 and SEQ ID NO: 222,

### (13) SEQ ID NO: 224 and SEQ ID NO: 226,

### (14) SEQ ID NO: 228 and SEQ ID NO: 230,

### (15) SEQ ID NO: 232 and SEQ ID NO: 234,

### (16) SEQ ID NO: 236 and SEQ ID NO: 238,

### (17) SEQ ID NO: 240 and SEQ ID NO: 242,

### (18) SEQ ID NO: 244 and SEQ ID NO: 246,

### (19) SEQ ID NO: 248 and SEQ ID NO: 250,

### (20) SEQ ID NO: 252 and SEQ ID NO: 254,

### (21) SEQ ID NO: 256 and SEQ ID NO: 258,

### (22) SEQ ID NO: 260 and SEQ ID NO: 262,

### (23) SEQ ID NO: 264 and SEQ ID NO: 266,

### (24) SEQ ID NO: 268 and SEQ ID NO: 270,

### (25) SEQ ID NO: 272 and SEQ ID NO: 274,

### (26) SEQ ID NO: 276 and SEQ ID NO: 278,

### (27) SEQ ID NO: 280 and SEQ ID NO: 282,

### (28) SEQ ID NO: 284 and SEQ ID NO: 286, and

### (29) SEQ ID NO: 288 and SEQ ID NO: 290.

[3] The antibody according to [1] or [2], which is an antibody fragment selected from the group consisting of Fab, Fab′, F(ab′)₂, a single chain antibody (scFv), a dimerized V region (Diabody), a disulfide stabilized V region (dsFv), and a peptide comprising CDR.
[4] DNA encoding the antibody according to any one of [1] to [3].
[5] A recombinant vector comprising the DNA according to [4].
[6] A transformed cell line obtained by introducing the recombinant vector according to [5] into a host cell.
[7] A method for producing the antibody according to any one of [1] to [3], which comprises culturing the transformed cell line according to [6] in a medium, then allowing the transformed cell line to generate and accumulate the antibody according to any one of [1] to [3] in a culture, and then collecting the antibody from the culture.
[8] A pharmaceutical composition comprising the antibody according to any one of [1] to [3].
[9] A passive immunotherapeutic agent against influenza, which comprises the antibody according to any one of [1] to [3].
[10] A passive immunotherapy against influenza, which comprises administering the antibody according to any one of [1] to [3] to a living body.
[11] The antibody according to any one of [1] to [3], which is used as a passive immunotherapeutic agent against influenza.
[12] Use of the antibody according to any one of [1] to [3] for production of a passive immunotherapeutic agent against influenza.

### Advantageous Effects of Invention

According to the present invention, a human antibody, which has universality as a single antibody against all influenza viruses belonging to Group 1, and also has a neutralizing activity sufficient for the treatment of humans, can be provided. In addition, an antibody capable of retaining a neutralizing activity also on influenza viruses belonging to Group 2 is also provided. That is to say, by passive immunization with the aforementioned neutralizing antibody, it becomes possible to effectively prevent or treat influenza with the antibody of the present invention, not only when an antigenic drift occurs, but also when currently unknown influenza, which will appear upon the occurrence of an antigen sift, becomes pandemic (for example, the pandemic of avian influenza).

### BRIEF DESCRIPTION OF DRAWINGS

FIG. 1 shows the schedule of vaccination and blood collection.

FIG. 2 shows the neutralization kinetic activity of representative clones.

### EMBODIMENTS FOR CARRYING OUT THE INVENTION

In the present description, the influenza virus includes the currently known subtypes of Group 1, and also subtypes, which will be isolated and identified in the future. The currently known influenza virus subtypes include subtypes each consisting of the combination of a type selected from hemagglutinin H1 to H16 and a type selected from neuraminidase N1 to N9.

The influenza virus is broadly divided into two groups depending on a difference in the antigenicity of hemagglutinin. In the present description, the group consisting of influenza viruses of types H1, H2, H5, H6, H8, H9, H11, H12, H13 and H16 is defined as Group 1, and the group consisting of influenza viruses of types H3, H4, H7, H10, H14 and H15 is defined as Group 2. Upon the category selection of these groups, the type of neuraminidase is not considered. Even regarding novel subtypes to be isolated and identified in the figure, the novel subtypes are classified into Group 1 or Group 2, depending on the similarity of the amino acid sequence of hemagglutinin.

The antibody of the present invention is preferably an antibody, which neutralizes at least one influenza virus selected from the aforementioned Group 1 (types H1, H2, H5, H6, H8, H9, H11, H12, H13 and H16) and at least one influenza virus selected from the aforementioned Group 2 (types H3, H4, H7, H10, H14 and H15). The neutralizing antibody of the present invention more preferably neutralizes at least H1 and/or H5 influenza viruses, among the influenza viruses of Group 1. The neutralizing antibody of the present invention particularly preferably neutralizes all of H1 to H16 influenza viruses.

The neutralizing antibody of the present invention can be produced by a method comprising the following steps (1) to (5):

(1) a step of providing an antibody library comprising antibody clones derived from approximately 10⁸ or more B cells collected from a single subject;
(2) a step of allowing influenza virus of any subtype of Group 1, a hemagglutinin protein of the virus, or an extracellular domain thereof to come into contact as an antigen with the antibody library of the step (1), and comprehensively selecting antibody clones that react with the antigen;
(3) a step of recovering an antibody molecule from each antibody clone selected in the step (2);
(4) a step of examining each antibody obtained in the step (3), in terms of its neutralizing activity on at least one influenza virus selected from Group 1 and at least one influenza virus selected from Group 2; and
(5) a step of allowing a clone that produces an antibody neutralizing influenza virus to produce the antibody, and then recovering the produced antibody.

The type of a donor, from which B cells serving as antibody-producing cells for production of an antibody library are collected, is not particularly limited, as long as it is any given mammal (e.g., a human, a swine, a horse, etc.) or any given avian (e.g., a chicken, a canard, etc.), which has been infected with influenza virus. Depending on a target to which the neutralizing antibody of the present invention is to be given by passive immunization, an animal of the same type as the target can be appropriately selected. The donor is preferably a human. In a case where the donor is a human, the age and sex of the donor, the presence or absence of vaccination, and the like are not particularly limited. Since it is desired for the donor to have been infected with influenza virus many times, the donor is preferably 20 years of age and older, more preferably 30 years of age and older, even more preferably 40 years of age and older, and particularly preferably 50 years of age and older, but the age of the donor is not limited thereto. A human who has not had an onset of influenza A for a certain period of time in the past is more desirable.

The amount of blood sampling for collection of B cells that are to be used in production of a common antibody library is approximately 20 to 30 mL, and the amount of B cells contained in this amount of blood is approximately 10⁷ cells. In all groups, from which a human neutralizing antibody reacting against highly pathogenic avian influenza virus H5N1 has been isolated, an ordinary amount of blood has been collected from each of a plurality of donors, and the collected blood samples have been then gathered to produce an antibody library of approximately 10¹⁰ clones. In contrast, the present inventors have intended to thoroughly (comprehensively) obtain antibodies that bind to hemagglutinin of a certain subtype, and have attempted to produce an antibody library with a size that reflects all antibody repertoires. That is, in general, the amount of blood collected from a human once is at most approximately 200 to 300 ml. Thus, the amount of B cells that can be collected by this method is at most approximately 10⁸ cells. Hence, the present inventors have collected from a single subject, B cells, which are contained in blood in an amount larger than the aforementioned amount, according to apheresis. The antibody library used for the purpose of the present invention is preferably constituted with 10⁹ or more B cells. For example, in order to collect approximately 10⁹ B cells from a single human body, B cells may be collected from approximately 3 L of blood according to apheresis.

Antibody-producing cells used to produce an antibody library may further comprise antibody-producing cells derived from another subject, as long as the cells contain approximately 10⁸ or more B cells, and preferably 10⁹ or more B cells, which are derived from the single subject. Specifically, monocytes corresponding to approximately 3 L of blood have been collected, for example, by apheresis, and thereafter, B cells can be isolated and recovered from the monocytes, for example, by Ficoll-Paque density gradient.

Examples of the antibody library include a phage display library, a library obtained by immortalization of B cells with EB virus, and a hybridoma library obtained by fusing B cells with myeloma cells, but the antibody library is not limited thereto. Preferably, a phage display library can be used.

Examples of the method for producing a phage display human antibody library in the present description include the following methods, but the production method is not limited thereto.

The type of the phage used herein is not particularly limited. In general, a filamentous phage (Ff bacteriophage) is preferably used. An example of a method of presenting a foreign protein on the surface of a phage is a method which comprises forming a fusion protein of a foreign protein and any coat protein of g3p (cp3) and g6p (cp6) to g9p (cp9), and allowing the foreign protein to express and/or present in the form of the fusion protein on the coat protein. A method of fusing a foreign protein with the N-terminal side of cp3 or cp8 has been often applied. Examples of a phage display vector include: 1) a vector, by which a foreign gene is introduced into a phage in a form in which the foreign gene is fused with a coat protein gene in the phage genome, and the coat protein is then presented on the surface of the phage in the form of a totally fusion protein with the foreign protein; 2) a vector, by which a gene encoding a fusion protein is inserted into a phage, separately from a wild-type coat protein gene, and allowing the fusion protein and the wild-type coat protein to simultaneously express on the surface of the phage; and 3) a vector, by which Escherichia coli comprising a phagemid vector having a gene encoding a fusion protein is infected with a helper phage having a wild-type coat protein gene, and phage particles simultaneously expressing the fusion protein and the wild-type coat protein are generated. In the case of 1) above, if a large foreign protein is fused with a coat protein, infectivity would be lost. In such a case, the vector 2) or 3) is used to produce an antibody library.

Examples of the specific vector include those described in Holt et al. (Curr. Opin. Biotechno., 11: 445-449, 2000). For example, pCES1 (see J. Biol. Chem., 274:18218-18230, 1999) is a Fab expression type phagemid vector, in which DNA encoding a kL chain constant region downstream of a cp3 signal peptide, DNA encoding C_{H3} downstream of a cp3 signal peptide, a His-tag, a C-myc tag, and a cp3 coding sequence chain via an amber stop codon (TAG) are disposed under the control of one lactose promoter. When this vector is introduced into Escherichia coli having an amber mutation, Fab is presented on a cp3 coat protein. However, when this vector is allowed to express in an HB2151 strain or the like, which does not have such an amber mutation, a soluble Fab antibody is generated. Moreover, as a scFv expression type phagemid vector, pHEN1 (J. Mol. Biol., 222:581-597, 1991) or the like is used.

On the other hand, examples of a helper phage include M13-K07 and VCSM13. Furthermore, another phage display vector, which has been prepared by ligating a sequence comprising a codon encoding cysteine to each of the 3′-end of an antibody gene and the 5′-end of a coat protein gene, and allowing the two genes to express simultaneously and separately (not as a fusion protein), so that it has been designed to present an antibody on the coat protein on the phage surface via an S—S bond caused by the introduced cysteine residues, is also used (CysDisplay™ technique of Morphosys).

Examples of the antibody library prepared in the present description include a naive/non-immunized library, a synthetic library, and an immunized library.

The naive/non-immunized library is a library prepared by obtaining V_{H} and V_{L} genes possessed by a normal animal by RT-PCR, and then randomly cloning these genes into the aforementioned phage display vector. In general, mRNA derived from lymphocytes from the peripheral blood, bone marrow, tonsil or the like of a normal animal (preferably, peripheral blood lymphocytes), or the like is used as a template. In order to eliminate the bias of the V gene, such as disease history, only the amplified mRNA derived from IgM, in which class switch caused by antigen sensitization does not occur, is particularly referred to as a “naive library.” Representative examples of such a naive library include the library of CAT (see J. Mol. Biol., 222: 581-597, 1991; Nat. Biotechnol., 14: 309-314, 1996), the library of MRC (see Annu. Rev. Immunol., 12: 433-455, 1994), and the library of Dyax (see J. Biol. Chom. (changed to “Chem.”), 1999 (as mentioned above); Proc. Natl. Acad. Sci. USA, 14: 7969-7974, 2000).

The synthetic library is prepared by selecting a specific functional antibody gene in human B cells, replacing a V gene fragment, for example, an antigen-binding region thereof close to CDR, with DNA encoding a random amino acid sequence having an appropriate length, and converting it to a library. Since such a library can be constituted by a combination of V_{H} and V_{L} genes generating originally functional scFv or Fab, this library is considered to be excellent in terms of antibody expression efficiency and stability. Representative examples of the synthetic library include the HuCAL library of Morphosys (see J. Mol. Biol., 296: 57-86, 2000), the library of Biolnvent (see Nat. Biotechnol, 18: 852, 2000), and the library of Crucell (see Proc. Natl. Acad. sci. USA, 92: 3938, 1995; J. Immumol. Methods, 272: 219-233, 2003). In the case of using such a synthetic library, it is desirable to use a VH1-69 or VH1-e gene fragment as a V gene fragment of the heavy chain variable region.

The immunized library is obtained by preparing mRNA from lymphocytes collected from a human whose blood antibody titer to a target antigen has been increased, such as a human who has received vaccination, or human lymphocytes, which have been artificially immunized with a target antigen by an in vitro immunization method, or the like, as in the case of the aforementioned naive/non-immunized library, then amplifying V_{H} and V_{L} genes by an RT-PCR method, and then converting it to a library. Since an antibody gene of interest is originally comprised in the library, an antibody of interest can be obtained even from a library with a relatively small size. However, antibodies specific to the subtypes of the virus immunized by vaccination are amplified. Thus, in the case of a human, if influenza viruses of hemagglutinin subtypes H1 to H3, regarding which a large number of antibodies reacting therewith are predicted to be present in the human body, are vaccinated to the human, it is likely that an antibody having a narrow range of neutralizing activity, which can neutralizes only specific isolated strains in the subtypes, would be amplified, and that a neutralizing antibody of interest would disappear. Accordingly, in the case of vaccination, it is preferable to use a vaccine for influenza virus of a subtype, regarding which a wide range of infection has not been reported so far (e.g., H5, H9, etc., in the case of a human).

As a diversity of libraries increases, it is better. However, in reality, taking into consideration the number of phages handled by the following panning operation (10¹¹ to 10¹³ phages) and the number of phages necessary for isolation and amplification of clones by an ordinary panning operation (100 to 1,000 phages/clone), approximately 10⁸ to 10¹¹ clones are appropriate. Preferably, 10⁹ clones and 10⁶ clones are suitable for V_{H} and V_{L} genes, respectively, and the number of Fab or scFV clones is 10¹⁰ to 10¹¹.

As an example of a method of preparing an antibody library by immortalization with EB virus, there is a method described in PLos Medicine 4 (5): e178 0928-9936 (2007), but the method is not limited thereto. A majority of people have been infected with EB virus as a subclinical infection of infectious mononucleosis, and thus, have been immunized with the EB virus. However, since viral particles are generated in the case of using ordinary EB virus, appropriate purification should be carried out. It is also preferable to use, as a system that does not involve virus contamination, a recombinant EB virus, which retains an ability to immortalize B lymphocytes but does not have an ability to replicate viral particles (e.g., a defect in a switch gene for transition from a latent infection condition to a lytic infection condition, etc.).

Since marmoset-derived B95-8 cells secrete EB virus, if a culture supernatant thereof were used, B lymphocytes could be easily transformed. These cells are cultured, for example, in a medium containing serum and penicillin/streptomycin (P/S) (e.g., RPMI 1640) or in a serum-free medium containing a cell growth factor, and a culture supernatant is then separated from the cultured cells by filtration, centrifugation, etc. Thereafter, antibody-producing B lymphocytes are suspended at a suitable concentration (e.g., approximately 10⁷ cells/mL) in the culture supernatant, and the obtained mixture is then incubated at a temperature of generally 20° C. to 40° C., and preferably 30° C. to 37° C., in general, for approximately 0.5 to 2 hours, so as to obtain antibody-producing B cell line. In a case where human antibody-producing cells are provided as mixed lymphocytes, since a majority of people have T lymphocytes exhibiting cytotoxicity to EB virus-infected cells, in order to increase transformation frequency, it is preferable to previously remove the T lymphocytes, for example, by forming an E rosette from the T lymphocytes and sheep erythrocytes, etc. In addition, soluble antigen-binding sheep erythrocytes are mixed with antibody-producing B lymphocytes, and a rosette is then separated by applying density gradient such as Percoll, so that lymphocytes specific to the target antigen can be selected. Moreover, by adding an excessive amount of antigen, antigen-specific B lymphocytes are capped, and they do not present IgG on the surface thereof. Thus, when they are mixed with anti-IgG antibody-binding sheep erythrocytes, only antigen-non-specific B lymphocytes form a rosette. Accordingly, by collecting a rosette-non-formation layer from the mixture by applying density gradient such as Percoll, antigen-specific B lymphocytes can be selected.

Antibody-secreting cells, which have obtained infinite proliferative capacity as a result of the transformation, can be fused again with mouse or human myeloma cells to stably maintain their antibody-secreting capacity. Examples of the mouse myeloma cells include NS-1, P3U1, SP2/0, and AP-1. Examples of the human myeloma cells include SKO-007, GM 1500-6TG-2, LICR-LON-HMy2, and UC729-6.

As a method of preparing an antibody library by cell fusion, preparation of hybridomas for general production of monoclonal antibodies can be applied. That is to say, B cells collected from a donor are fused with the aforementioned myeloma cells to prepare antibody-producing hybridomas.

The fusion operation can be carried out by a known method, for example, the method of Kohler and Milstein [Nature, Vol. 256, p. 495 (1975)]. Examples of a fusion promoter include polyethylene glycol (PEG) and Sendai virus. Preferably, PEG or the like is used. The molecular weight of PEG is not particularly limited. PEG1000 to PEG6000, which are low toxic and have a relatively low viscosity, are preferable. The concentration of PEG is, for example, approximately 10% to 80%, and preferably approximately 30% to 50%. As a solution for diluting PEG, a serum-free medium (e.g., RPMI 1640), a complete medium containing approximately 5% to 20% serum, various types of buffers such as a phosphate buffered saline (PBS) or a Tris buffer, can be used. As desired, DMSO (e.g., approximately 10% to 20%) can also be added. The pH of the fused solution is, for example, approximately 4 to 10, and preferably, approximately 6 to 8.

The preferred ratio of the number of B cells and the number of myeloma cells is generally approximately 1:1 to 20:1, and cell fusion can be efficiently carried out by incubating these cells at a temperature of generally 20° C. to 40° C., preferably 30° C. to 37° C., and generally for 1 to 10 minutes.

The screening and breeding of hybridomas are generally carried out in a medium for animal cells (e.g., RPMI 1640) containing 5% to 20% FCS, or a cell growth factor-containing serum-free medium, to which HAT (hypoxanthine, aminopterin, and thymidine) has been added. The concentrations of hypoxanthine, aminopterin, and thymidine are, for example, approximately 0.1 mM, approximately 0.4 μM, and approximately 0.016 mM, respectively. For selection of human or mouse hybridomas, ouabain resistance can be used. Since a human cell line has higher sensitivity to ouabain than a mouse cell line, by adding ouabain at a concentration of approximately 10⁻⁷ to 10⁻³ M to the medium, unfused human cells can be eliminated.

For selection of hybridomas, it is preferable to use feeder cells or a certain type of cell culture supernatant. As such feeder cells, allogeneic cell species having a limited survival period, which help the appearance of hybridomas and die by themselves, or cells capable of producing a large amount of growth factors useful for the appearance of hybridomas, the proliferation potency of which has been reduced by radiation exposure or the like, etc. can be used Examples of the mouse feeder cells include splenic cells, macrophages, blood, and thymocytes. Examples of the human feeder cells include peripheral blood monocytes. Examples of the cell culture supernatant include the primary culture supernatants of various types of the aforementioned cells, and the culture supernatants of various established cells.

A step of selecting an antibody reacting with a target antigen by a phage display method is referred to as “panning.” Specifically, a carrier, on which influenza virus of any subtype in Group 1, or a hemagglutinin protein of the virus, or an extracellular domain thereof is immobilized, is allowed to come into contact with a phage library, and non-bound phages are then removed by washing. Thereafter, the bound phages are eluted from the carrier, and are then allowed to infect Escherichia coli, so that the phages are allowed to proliferate therein. By repeating a series of the operations about two to four times, phages presenting antigen-specific antibodies are concentrated. In the case of the present invention, influenza virus serving as an antigen may be inactivated by a treatment with formalin.

The cDNA sequence encoding hemagglutinin of each subtype is known, and recombinant hemagglutinin of a desired subtype can be produced by applying a common genetic recombination technique. Moreover, the trimer extracellular domain structure of hemagglutinin can be prepared according to the method described in Nature, Vol. 289, PP. 373-378, 1981.

Examples of a carrier, on which an antigen is immobilized, include various types of carriers used in general antigen-antibody reactions or affinity chromatography, including insoluble polysaccharides such as agarose, dextran or cellulose, synthetic resins such as polystyrene, polyacrylamide or silicone a microplate consisting of glass or metal, a tube, a membrane, a column, beads, and a sensor chip of surface plasmon resonance (SPR). For immobilization of an antigen, physical adsorption may be used, or a method involving chemical binding, which is generally used to insolubilize or immobilize a protein, enzyme or the like, may also be used. For example, a biotin-(strept)avidin system or the like is preferably used. For the washing of non-bound phages, a blocking solution such as a BSA solution (once or twice), PBS containing a surfactant such as Tween (three to five times), and the like can be successively used. It has also been reported that the use of a citrate buffer (PHS) or the like is preferable. For elution of specific phages, an acid (e.g., 0.1 M hydrochloric acid, etc.) is generally used. It is also possible to carry out cleavage with specific protease (For example, a gene sequence encoding a trypsin cleavage site can be introduced into a connected portion between an antibody gene and a coat protein gene. In this case, since a wild-type coat protein is presented on the surface of the eluted phage, even if all of the coat protein is expressed as a fusion protein, infection to Escherichia coli and/or proliferation become possible.), competitive elution with a soluble antigen, or elution by reduction of S—S bond (For example, in the aforementioned CySDisplay™, after completion of the panning, the antibody is dissociated from the coat protein using a suitable reducing agent, so that an antigen-specific phage can be recovered.) can also be carried out. In the case of elution with an acid, the eluted phage is neutralized with Tris buffer or the like, and it is then allowed to infect Escherichia coli, and after completion of the culture, phages are recovered according to an ordinary method.

It is also possible that a hemagglutinin trimer is allowed to express on a cell membrane using an enzyme display, instead of immobilizing an antigen on a carrier.

After phages presenting an antigen-specific antibody have been concentrated by panning, these phages are allowed to infect Escherichia coli, and the Escherichia coli are then inoculated on a plate, followed by cloning. The phages are recovered again, and the antigen-binding activity is then measured by using an antibody titer measurement method (e.g., ELISA, RIA, NA, etc.), FACS (fluorescence activated cell sorting) or SPR (Surface Plasmon Resonance).

With regard to a step of allowing the above-obtained phage antibody clone to infect Escherichia coli, and then recovering an antibody from a culture supernatant, for example, in the case of using a phage display vector in which an amber stop codon is introduced into a connected portion between an antibody gene and a coat protein gene, if the phage is allowed to infect Escherichia coli having no amber mutations (e.g., the strain HB2151), soluble antibody molecules are generated, and they are then secreted into a periplasm or a medium. Accordingly, the cell wall is solubilized with lysozyme or the like, and an extracellular fraction is recovered, and it can be then purified using the same purification technique as described above. If a His-tag or a c-myc tag has been previously introduced, the fraction can be easily purified using IMAC or an anti-c-myc antibody column. Moreover, if cleavage with specific protease is used for panning, since an antibody molecule is separated from the surface of a phage by the action of the protease, the same purification operation as described above is carried out, so as to purify an antibody of interest. In the case of the present invention, the neutralizing activity of an IgG-type complete antibody is approximately 100 to 1,000 times higher than that of a Fab-type antibody. Thus, as described in the Examples later, plasmid DNA is recovered from the obtained phage clone, a sequence corresponding to a domain binding to the Fe of IgG is then added thereto by genetic engineering, and Escherichia coli is then transformed with the resulting DNA, followed by culture. Thereafter, an antibody recovered from the culture supernatant is purified with an IgG Sepharose column, and it is then subjected to the test of neutralizing activity.

As a method of selecting an antibody of interest from an antibody-producing cell line obtained by immortalization with EB virus or cell fusion, for example, the aforementioned antigen is fluorescently labeled, and is then allowed to react with immortalized cells or fusion cells, and thereafter, cells binding to the antigen are separated from the resulting cells using a fluorescence-activated cell sorter (FACS), so as to select the antibody of interest. In this case, since hybridomas, which produce antibodies binding to target antigens or immortalized B cells, can be directly selected, it is possible to greatly reduce efforts for cloning.

For the cloning of hybridomas producing monoclonal antibodies binding to target antigens, various methods can be applied.

Since aminopterin inhibits many cellular functions, it is preferable to remove it from the medium as soon as possible. However, human hybridomas are generally maintained in an aminopterin-added medium for approximately 4 to 6 weeks after cell fusion. Hypoxanthine and thymidine are desirably removed from the medium one or more weeks after the removal of aminopterin. After a clone has appeared and the diameter thereof has become approximately 1 mm, it becomes possible to measure the amount of the antibody in the culture supernatant.

The amount of an antibody can be measured, for example, by a method which comprises adding a culture supernatant of hybridomas to a solid phase (e.g., a microplate) on which a target antigen, a derivative thereof, or a partial peptide thereof is adsorbed directly or together with a carrier, then adding an anti-immunoglobulin IgG antibody (which is an antibody reacting with IgG derived from an animal of the same species as the animal, from which the original antibody-producing cells have been derived) or protein A, which has been labeled with a radioactive substance (e.g., ¹²⁵I, ¹³¹I, ³H, or ¹⁴C), enzyme (e.g., β-galactosidase, β-glucosidase, alkaline phosphatase, peroxidase, or malate dehydrogenase), a fluorescent substance (e.g., fluorescamine, fluorescein isothiocyanate), a luminescent substance (e.g., luminol, a luminol derivative, luciferin, or lucigenin) or the like, and then detecting an antibody reacting with the target antigen bound to the solid phase, or a method which comprises adding a culture supernatant of hybridomas to a solid phase, on which an anti-IgG antibody or protein A has been adsorbed, then adding a target antigen, a derivative thereof, or a partial peptide thereof, which has been labeled with the same labeling agent as described above to the solid phase, and then detecting an antibody reacting with the target antigen bound to the solid phase.

As a cloning method, a limiting dilution method is generally used. In addition, cloning using soft agar or cloning involving FACS (as described above) can also be applied. Cloning involving a limiting dilution method can be carried out, for example, by the following procedures, but is not limited thereto.

The amount of an antibody is measured as described above, and positive wells are then selected. Suitable feeder cells are selected, and are then added to a 96-well plate. Cells are sucked out of the antibody-positive cells, and they are then suspended at a density of 30 cells/mL in a complete medium (e.g., RPMI 1640 containing 10% FCS (Fetal Bovine/Calf Serum) and P/S). Thereafter, 0.1 mL (3 cells/well) of the cell suspension is added to the well plate, to which the feeder cells have been added. The remaining cell suspension is diluted to a density of 10 cells/mL, and it is then dispersed on another well (1 cell/well) in the same manner as described above. The further remaining cell suspension is diluted to a density of 3 cells/mL, and it is then dispersed on another well (0.3 cell/well). The cells are cultured for approximately 2 to 3 weeks, until visible clones have appeared, and thereafter, the amount of the antibody is measured. Positive wells are selected, and are then cloned again. Since it is relatively difficult to clone human cells, a plate containing the cell suspension at a density of 10 cells/well is also prepared. In general, monoclonal antibody-producing hybridomas can be obtained by two times of subcloning operations. In order to confirm the stability thereof, it is desired to regularly carry out re-cloning operations for further several months.

Hybridomas can be cultured in vitro or in vivo.

An example of the in vitro culture method is a method of gradually scaling-up the above-obtained monoclonal antibody-producing hybridomas from the well plate, while the cell density is maintained at approximately 10⁵ to 10⁶ cells/mL, and while the FCS concentration is gradually decreased.

An example of the in vivo culture method is a method which comprises injecting mineral oil into the abdominal cavity of a mouse (which is tissue compatible with the parent strain of hybridomas) to induce plasmocytoma (MOPC), then 5 to 10 days later, intraperitoneally injecting 10⁶ to 10⁷ hybridomas to the mouse, and then 2 to 5 weeks later, collecting ascites from the mouse under anesthesia.

As with general separation and purification of a polyclonal antibody, separation and purification of a monoclonal antibody are carried out by an immunoglobulin separation purification method [e.g., a salting-out method, an alcohol precipitation method, an isoelectric point precipitation method, electrophoresis, an adsorption-desorption method using an ion exchanger (e.g., DEAE and QEAE), an ultracentrifugation method, a gel filtration method, a specific purification method which comprises collecting only the antibody using an antigen-bound solid phase or an active adsorbent such as protein A or protein and then dissociating the bond to obtain the antibody, etc.].

As stated above, hybridomas are cultured in vivo or ex vivo of a warm-blooded animal, and an antibody is then collected from the body fluid or culture thereof, so that a monoclonal antibody binding to influenza virus of a specific hemagglutinin subtype can be screened.

An example of the method of testing the neutralizing activity in the present invention is a focus formation inhibition test (J. Clin. Miorobiol. Vol. 28, PP 1308-1313 (1990)). That is to say, influenza virus is allowed to come into contact with host cells in the presence or absence of a test antibody, and based on whether or not focus formation caused by viral infection to the host cells is significantly inhibited by the test antibody, the presence or absence of a neutralizing activity and the degree thereof are determined.

The subtype of influenza virus, regarding which the neutralizing activity of a test antibody is to be examined, is not particularly limited. Such influenza virus subtypes preferably include at least H1 influenza virus from Group 1.

Using a clone that produces an antibody molecule that has been confirmed to neutralize at least one influenza virus selected from Group 1 as a result of the neutralizing activity test, the antibody molecule can be produced in a large amount. When the used antibody library is a phage display library, a phage clone presenting Fab or scFv of the neutralizing antibody of interest is allowed to infect Escherichia coli, and the Escherichia coli is then cultured, so that a Fab-type antibody or a scFv-type antibody may be prepared. However, for the purpose of significantly increasing the neutralizing activity, it is more preferable to convert these antibodies to IgG-type antibodies. For example, Fab can be converted to IgG by cutting fragments encoding VHCH1 and VLCL from phage DNA, inserting them into a plasmid comprising a fragment encoding an Fc region so as to construct a plasmid comprising DNA encoding a heavy chain and a light chain thereof, then transfecting animal cells such as CHO cells with this plasmid, followed by performing a culture, and then allowing an IgG-type antibody to secrete into the culture supernatant. The obtained antibody can be purified and/or recovered by a known method.

Moreover, when the used antibody library consists of EB virus-immortalized B cells or hybridomas obtained by cell fusion, an antibody molecule is produced in vitro or in vivo, as described above, and it is then purified by an ordinary method, so as to recover the antibody.

The affinity of the obtained neutralizing antibody for an antigen can be enhanced in vitro by imitating the steps adopted by an immune system (somatic mutation and selection). Examples of a method of introducing a mutation into an antibody gene include a chain shuffling method, random mutagenesis involving Escherichia coli that is easily mutated as a result of deficiency in a repair system thereof or error-prone PCR, and CDR walking. A neutralizing antibody with an improved affinity for an antigen can be selected by screening for a high-affinity neutralizing antibody through a mutation library prepared by mutation introduction. Examples of the method that can be used herein include: 1) a method of setting the concentration of an antigen used for selection at low and recovering a high-affinity phage; 2) a method of setting strict washing conditions and recovering an antibody phage that is hardly removed from an antigen; and 3) a method of using an antagonistic reaction.

A majority of the neutralizing antibodies of the present invention, which have been obtained as described above, utilize a VH1-69 or VH1-e gene as a heavy chain V region. This is a characteristic that is shared by various antibodies neutralizing influenza viruses of a plurality of subtypes in Group 1, which has been reported so far.

When the characteristics of the previously reported neutralizing antibodies against human influenza are verified at a monoclonal antibody level, the target epitope is the head of influenza hemagglutinin (HA) of the influenza, and in general, the antibodies have a neutralizing activity only on specific strains and some subtype strains having high homology with the specific strains. This is because the antigenicity and immunogenicity of influenza to human are extremely high at the head of HA, and mutation sites have been concentrated to the HA during the evolutional process of influenza.

Accordingly, in order to obtain a versatile neutralizing antibody having a wide neutralizing ability capable of coping with continuous mutations in a single subtype, it is effective to produce an antibody reacting with a portion in which antigenicity and immunogenicity are considered to be low.

In the present invention, the inventors have produced an antibody reacting with a stem portion, in which a mutation hardly occurs, and have succeeded in making an invention relating to an antibody capable of coping with not only continuous mutations in a single subtype but also discontinuous mutations over several subtypes. Previously, the present inventors had produced an antibody reacting with a sialic acid pocket, which is only a site to be hardly mutated in the HA head of influenza, and thus, the inventors had succeeded in producing a monoclonal antibody having a neutralizing activity on viruses ranging over several subtypes (Patent Literature 3, Ohshima N et al., 2011 J Virol 85 (21)).

In the case of an antibody, during the differentiation process of B cells, reorganization of an immunoglobulin gene, namely, recombination of V, D and J regions of a heavy chain variable domain or recombination of V and J regions of a light chain variable domain occurs, and thereafter, a somatic cell mutation is induced to the nucleotide sequence of a variable region. As a result, an antibody having a variable domain having a higher affinity for an antigen can be produced. Accordingly, the neutralizing antibody of the present invention, which is an antibody clone derived from B cells, may also include a neutralizing antibody having an amino acid sequence comprising a somatic cell mutation of the original immunoglobulin gene. Moreover, upon formation of an antigen-antibody complex of a neutralizing antibody and an influenza virus antigen, the contact points with hemagglutinin molecules are all heavy chain variable domains Thus, a site substantially contributing to the affinity of the neutralizing antibody for influenza virus has been anticipated to be a heavy chain variable domain. Furthermore, since the complementarity-determining region 3 (CDR 3) does not contribute so much to the binding of the neutralizing antibody of the present invention with an antigen, the complementarity-determining regions 1 and 2, which are present in the heavy chain V region, are more important. In the present description, the term “heavy chain variable domain V region (light chain variable domain V region)” is used to mean a V region constituting the variable domain of a heavy chain (light chain) after completion of the reorganization, and it may be, for example, a region including the framework regions 1, 2 and 3 and the complementarity-determining regions 1 and 2. Further, the heavy chain (light chain) variable domain indicates a domain in the Fab region, which is not a constant domain, and it may be, for example, a region including the framework regions 1, 2 and 3 and the complementarity-determining regions 1, 2 and 3.

Specific examples of the present invention include antibodies, the heavy chain variable domain and the light chain variable domain of which consist of amino acid sequences shown in the amino acid sequence numbers described in any one of the following (1) to (29):

### (1) VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5, and VL CDR3 of SEQ ID NO: 6,

### (2) VH CDR1 of SEQ ID NO: 7, VH CDR2 of SEQ ID NO: 8, VH CDR3 of SEQ ID NO: 9, VL CDR1 of SEQ ID NO: 10, VL CDR2 of SEQ ID NO: 11, and VL CDR3 of SEQ ID NO: 12,

### (3) VH CDR1 of SEQ ID NO: 13, VH CDR2 of SEQ ID NO: 14, VH CDR3 of SEQ ID NO: 15, VL CDR1 of SEQ ID NO: 16, VL CDR2 of SEQ ID NO: 17, and VL CDR3 of SEQ ID NO: 18,

### (4) VH CDR1 of SEQ ID NO: 19, VH CDR2 of SEQ ID NO: 20, VH CDR3 of SEQ ID NO: 21, VL CDR1 of SEQ ID NO: 22, VL CDR2 of SEQ ID NO: 23, and VL CDR3 of SEQ ID NO: 24,

### (5) VH CDR1 of SEQ ID NO: 25, VH CDR2 of SEQ ID NO: 26, VH CDR3 of SEQ ID NO: 27, VL CDR1 of SEQ ID NO: 28, VL CDR2 of SEQ ID NO: 29, and VL CDR3 of SEQ ID NO: 30,

### (6) VH CDR1 of SEQ ID NO: 31, VH CDR2 of SEQ ID NO: 32, VH CDR3 of SEQ ID NO: 33, VL CDR1 of SEQ ID NO: 34, VL CDR2 of SEQ ID NO: 35, and VL CDR3 of SEQ ID NO: 36,

### (7) VH CDR1 of SEQ ID NO: 37, VH CDR2 of SEQ ID NO: 38, VH CDR3 of SEQ ID NO: 39, VL CDR1 of SEQ ID NO: 40, VL CDR2 of SEQ ID NO: 41, and VL CDR3 of SEQ ID NO: 42,

### (8) VH CDR1 of SEQ ID NO: 43, VH CDR2 of SEQ ID NO: 44, VH CDR3 of SEQ ID NO: 45, VL CDR1 of SEQ ID NO: 46, VL CDR2 of SEQ ID NO: 47, and VL CDR3 of SEQ ID NO: 48,

### (9) VH CDR1 of SEQ ID NO: 49, VH CDR2 of SEQ ID NO: 50, VH CDR3 of SEQ ID NO: 51, VL CDR1 of SEQ ID NO: 52, VL CDR2 of SEQ ID NO: 53, and VL CDR3 of SEQ ID NO: 54,

### (10) VH CDR1 of SEQ ID NO: 55, VH CDR2 of SEQ ID NO: 56, VH CDR3 of SEQ ID NO: 57, VL CDR1 of SEQ ID NO: 58, VL CDR2 of SEQ ID NO: 59, and VL CDR3 of SEQ ID NO: 60,

### (11) VH CDR1 of SEQ ID NO: 61, VH CDR2 of SEQ ID NO: 62, VH CDR3 of SEQ ID NO: 63, VL CDR1 of SEQ ID NO: 64, VL CDR2 of SEQ ID NO: 65, and VL CDR3 of SEQ ID NO: 66,

### (12) VH CDR1 of SEQ ID NO: 67, VH CDR2 of SEQ ID NO: 68, VH CDR3 of SEQ ID NO: 69, VL CDR1 of SEQ ID NO: 70, VL CDR2 of SEQ ID NO: 71, and VL CDR3 of SEQ ID NO: 72,

### (13) VH CDR1 of SEQ ID NO: 73, VH CDR2 of SEQ ID NO: 74, VH CDR3 of SEQ ID NO: 75, VL CDR1 of SEQ ID NO: 76, VL CDR2 of SEQ ID NO: 77, and VL CDR3 of SEQ ID NO: 78,

### (14) VH CDR1 of SEQ ID NO: 79, VH CDR2 of SEQ ID NO: 80, VH CDR3 of SEQ ID NO: 81, VL CDR1 of SEQ ID NO: 82, VL CDR2 of SEQ ID NO: 83, and VL CDR3 of SEQ ID NO: 84,

### (15) VH CDR1 of SEQ ID NO: 85, VH CDR2 of SEQ ID NO: 86, VH CDR3 of SEQ ID NO: 87, VL CDR1 of SEQ ID NO: 88, VL CDR2 of SEQ ID NO: 89, and VL CDR3 of SEQ ID NO: 90,

### (16) VH CDR1 of SEQ ID NO: 91, VH CDR2 of SEQ ID NO: 92, VH CDR3 of SEQ ID NO: 93, VL CDR1 of SEQ ID NO: 94, VL CDR2 of SEQ ID NO: 95, and VL CDR3 of SEQ ID NO: 96,

### (17) VH CDR1 of SEQ ID NO: 97, VH CDR2 of SEQ ID NO: 98, VH CDR3 of SEQ ID NO: 99, VL CDR1 of SEQ ID NO: 100, VL CDR2 of SEQ ID NO: 101, and VL CDR3 of SEQ ID NO: 102,

### (18) VH CDR1 of SEQ ID NO: 103, VH CDR2 of SEQ ID NO: 104, VH CDR3 of SEQ ID NO: 105, VL CDR1 of SEQ ID NO: 106, VL CDR2 of SEQ ID NO: 107, and VL CDR3 of SEQ ID NO: 108,

### (19) VH CDR1 of SEQ ID NO: 109, VH CDR2 of SEQ ID NO: 110, VH CDR3 of SEQ ID NO: 111, VL CDR1 of SEQ ID NO: 112, VL CDR2 of SEQ ID NO: 113, and VL CDR3 of SEQ ID NO: 114,

### (20) VH CDR1 of SEQ ID NO: 115, VH CDR2 of SEQ ID NO: 116, VH CDR3 of SEQ ID NO: 117, VL CDR1 of SEQ ID NO: 118, VL CDR2 of SEQ ID NO: 119, and VL CDR3 of SEQ ID NO: 120,

### (21) VH CDR1 of SEQ ID NO: 121, VH CDR2 of SEQ ID NO: 122, VH CDR3 of SEQ ID NO: 123, VL CDR1 of SEQ ID NO: 124, VL CDR2 of SEQ ID NO: 125, and VL CDR3 of SEQ ID NO: 126,

### (22) VH CDR1 of SEQ ID NO: 127, VH CDR2 of SEQ ID NO: 128, VH CDR3 of SEQ ID NO: 129, VL CDR1 of SEQ ID NO: 130, VL CDR2 of SEQ ID NO: 131, and VL CDR3 of SEQ ID NO: 132,

### (23) VH CDR1 of SEQ ID NO: 133, VH CDR2 of SEQ ID NO: 134, VH CDR3 of SEQ ID NO: 135, VL CDR1 of SEQ ID NO: 136, VL CDR2 of SEQ ID NO: 137, and VL CDR3 of SEQ ID NO: 138,

### (24) VH CDR1 of SEQ ID NO: 139, VH CDR2 of SEQ ID NO: 140, VH CDR3 of SEQ ID NO: 141, VL CDR1 of SEQ ID NO: 142, VL CDR2 of SEQ ID NO: 143, and VL CDR3 of SEQ ID NO: 144,

### (25) VH CDR1 of SEQ ID NO: 145, VH CDR2 of SEQ ID NO: 146, VH CDR3 of SEQ ID NO: 147, VL CDR1 of SEQ ID NO: 148, VL CDR2 of SEQ ID NO: 149, and VL CDR3 of SEQ ID NO: 150,

### (26) VH CDR1 of SEQ ID NO: 151, VH CDR2 of SEQ ID NO: 152, VH CDR3 of SEQ ID NO: 153, VL CDR1 of SEQ ID NO: 154, VL CDR2 of SEQ ID NO: 155, and VL CDR3 of SEQ ID NO: 156,

### (27) VH CDR1 of SEQ ID NO: 157, VH CDR2 of SEQ ID NO: 158, VH CDR3 of SEQ ID NO: 159, VL CDR1 of SEQ ID NO: 160, VL CDR2 of SEQ ID NO: 161, and VL CDR3 of SEQ ID NO: 162,

### (28) VH CDR1 of SEQ ID NO: 163, VH CDR2 of SEQ ID NO: 164, VH CDR3 of SEQ ID NO: 165, VL CDR1 of SEQ ID NO: 166, VL CDR2 of SEQ ID NO: 167, and VL CDR3 of SEQ ID NO: 168, and

### (29) VH CDR1 of SEQ ID NO: 169, VH CDR2 of SEQ ID NO: 170, VH CDR3 of SEQ ID NO: 171, VL CDR1 of SEQ ID NO: 172, VL CDR2 of SEQ ID NO: 173, and VL CDR3 of SEQ ID NO: 174.

Moreover, with regard to the antibodies described in the above (1) to (29), the nucleotide sequences and amino acid sequences of individual heavy chains and light chains are shown in the following Table 1.

TABLE 1

| | | Nucleotide | Amino acid | Nucleotide | Amino acid |
|---|---|---|---|---|---|
| | | sequence of | sequence of | sequence of | sequence of |
| ID | Antibody | heavy chain | heavy chain | light chain | light chain |
| F080-227 | 1 | SEQ ID NO: 175 | SEQ ID NO: 176 | SEQ ID NO: 177 | SEQ ID NO: 178 |
| F081-007 | 2 | SEQ ID NO: 179 | SEQ ID NO: 180 | SEQ ID NO: 181 | SEQ ID NO: 182 |
| F081-008 | 3 | SEQ ID NO: 183 | SEQ ID NO: 184 | SEQ ID NO: 185 | SEQ ID NO: 186 |
| F081-107 | 4 | SEQ ID NO: 187 | SEQ ID NO: 188 | SEQ ID NO: 189 | SEQ ID NO: 190 |
| F081-264 | 5 | SEQ ID NO: 191 | SEQ ID NO: 192 | SEQ ID NO: 193 | SEQ ID NO: 194 |
| F081-281 | 6 | SEQ ID NO: 195 | SEQ ID NO: 196 | SEQ ID NO: 197 | SEQ ID NO: 198 |
| F082-117 | 7 | SEQ ID NO: 199 | SEQ ID NO: 200 | SEQ ID NO: 201 | SEQ ID NO: 202 |
| F082-220 | 8 | SEQ ID NO: 203 | SEQ ID NO: 204 | SEQ ID NO: 205 | SEQ ID NO: 206 |
| F082-237 | 9 | SEQ ID NO: 207 | SEQ ID NO: 208 | SEQ ID NO: 209 | SEQ ID NO: 210 |
| F082-243 | 10 | SEQ ID NO: 211 | SEQ ID NO: 212 | SEQ ID NO: 213 | SEQ ID NO: 214 |
| F082-338 | 11 | SEQ ID NO: 215 | SEQ ID NO: 216 | SEQ ID NO: 217 | SEQ ID NO: 218 |
| F083-103 | 12 | SEQ ID NO: 219 | SEQ ID NO: 220 | SEQ ID NO: 221 | SEQ ID NO: 222 |
| F083-115 | 13 | SEQ ID NO: 223 | SEQ ID NO: 224 | SEQ ID NO: 225 | SEQ ID NO: 226 |
| F083-249 | 14 | SEQ ID NO: 227 | SEQ ID NO: 228 | SEQ ID NO: 229 | SEQ ID NO: 230 |
| F083-301 | 15 | SEQ ID NO: 231 | SEQ ID NO: 232 | SEQ ID NO: 233 | SEQ ID NO: 234 |
| F083-302 | 16 | SEQ ID NO: 235 | SEQ ID NO: 236 | SEQ ID NO: 237 | SEQ ID NO: 238 |
| F083-305 | 17 | SEQ ID NO: 239 | SEQ ID NO: 240 | SEQ ID NO: 241 | SEQ ID NO: 242 |
| F083-307 | 18 | SEQ ID NO: 243 | SEQ ID NO: 244 | SEQ ID NO: 245 | SEQ ID NO: 246 |
| F083-308 | 19 | SEQ ID NO: 247 | SEQ ID NO: 248 | SEQ ID NO: 249 | SEQ ID NO: 250 |
| F083-311 | 20 | SEQ ID NO: 251 | SEQ ID NO: 252 | SEQ ID NO: 253 | SEQ ID NO: 254 |
| F083-318 | 21 | SEQ ID NO: 255 | SEQ ID NO: 256 | SEQ ID NO: 257 | SEQ ID NO: 258 |
| F083-328 | 22 | SEQ ID NO: 259 | SEQ ID NO: 260 | SEQ ID NO: 261 | SEQ ID NO: 262 |
| F083-336 | 23 | SEQ ID NO: 263 | SEQ ID NO: 264 | SEQ ID NO: 265 | SEQ ID NO: 266 |
| F083-349 | 24 | SEQ ID NO: 267 | SEQ ID NO: 268 | SEQ ID NO: 269 | SEQ ID NO: 270 |
| F083-351 | 25 | SEQ ID NO: 271 | SEQ ID NO: 272 | SEQ ID NO: 273 | SEQ ID NO: 274 |
| F083-354 | 26 | SEQ ID NO: 275 | SEQ ID NO: 276 | SEQ ID NO: 277 | SEQ ID NO: 278 |
| F083-366 | 27 | SEQ ID NO: 279 | SEQ ID NO: 280 | SEQ ID NO: 281 | SEQ ID NO: 282 |
| F083-373 | 28 | SEQ ID NO: 283 | SEQ ID NO: 284 | SEQ ID NO: 285 | SEQ ID NO: 286 |
| F083-389 | 29 | SEQ ID NO: 287 | SEQ ID NO: 288 | SEQ ID NO: 289 | SEQ ID NO: 290 |

The amino acid sequences of the heavy chains of the above-mentioned 29 types of antibodies of the present invention are shown in Table 2, and the amino acid sequences of the light chains thereof are shown in Table 3.

TABLE 2

| clone | | VH FR1 | VH CDR1 | VH FR2 | VH CDR2 |
|---|---|---|---|---|---|
| F080-227 | QVQLQESGAE | VKKPGSSVKVSCKASGGIFR | RNAIS | WVRQAPGQLGEWMG | GIIAIFGTANYAQKFQG |
| F081-007 | QVQLQQSGAE | VKKPGSSVKVSCKASGGIFR | RNAIS | WVRQAPGQLGEWMG | GISAIFGIANYAQKFQG |
| F081-008 | QVQLVQSGAE | VKKPGSSVKVSCKASGGIFS | SFSTYTIS | WVRQAPGQLGEWMG | RSIPLLGITNYAQKFQG |
| F081-107 | QVQLVQSGAE | VKKPGSSVKVSCKASGGIFR | KNAIS | YVRQAPGQGVEWMG | GITAIFGTPNYAQKFQG |
| F081-264 | EVQLVESGAE | VKKPGSSVKVSCKASGGHFN | AYGIS | WVRQAPGQLGEWMG | GIVALFGTTNYAQKLQG |
| F081-281 | QVQLVQSGSE | VKKPGSSVKVSCSFKASGGIIS | KYAIT | WVRQAPGQLGEWMG | GIIAIFGSTNYAQKFQG |
| F082-117 | QVQLVQSGSE | VKKPGSSVKVSCKSSGGILR | RSAIS | WVRQAPGQLGEWMG | GILAIFGTTKYAQKRQG |
| F082-220 | QVQLVQSGAE | VKKPGSSVKVSCKASGGIRF | SNAVS | WVRQAPGQLGEWMG | GIIAIFGIPKYAQKFQG |
| F082-237 | QVQLVQSGAE | VKKPGSSVKVSCKASGGIFN | KYGIS | WVRQAPGQLGEWMG | GILAIFGTTNYAQKRQG |
| F082-243 | QVQLQQSGAE | VKKPGSSVKVSCKASGGIFR | KNAIS | WVRQAPGQLGEWMG | GIIAIFGTANYAQKFQG |
| F082-338 | QVQLQESGAE | VKKPGSSVKVSCKASGGPFR | SHAIS | WVRQAPGQLGEWMG | GIIAVFGTANYAQKFQG |
| F083-103 | QVQLQQSGAD | VKKAGSSVKVSCKASGRTFG | NYAIS | WVRQAPGQLGEWMG | GIIPIFGAANYAQKRQG |
| F083-115 | QVQLVQSGAE | VKKPGSSVKVSCKASGGIFR | KSAIT | WVRQAPGQLGEWMG | GIIAIFGTTNYAQKFQD |
| F083-249 | QVQLVQSGSE | VKKPGSSVKVSCKASGVTGS | SYAIS | WVRQAPGQLGEWMG | GISPMFGTTRYAQKFQG |
| F083-301 | QVQLVQSGAE | VKKPGSSVKVSCKASGGIFN | SNAIS | WVRQAPGQLGEWMG | GIIGIFGTANYAQKRQG |
| F083-302 | QVQLVGSGAE | VKKPGSSVKVSCEASGATES | SNAFS | WVRQAPGQLGEWMG | GIITMFRKAEYAQKFQG |
| F083-305 | EVQLVESGAE | VKKPGSSVKVSCKASGSIFS | NYAIS | WVRQAPGQLGEWMG | GIVPMFGTTRFAQKFQG |
| F083-307 | QVQLQQSGAD | VKKPGSSVKVSCKASGGTFG | NYAIS | WVRQAPGQLGEWMG | GITPIFGPANYAQRFQG |
| F083-308 | QVQLVQSGAE | VKKPGSSVKVSCKASGGIFR | SHAIS | WVRQAPGQLGEWMG | GIIAIFGTTHYAQQFQG |
| F083-311 | QVQLVQSGSE | VKKPGSSVKVSCKASGGIFR | INAIS | YVRQAPGQGLEWMG | GITAIFGTPNYAQKFQG |
| F083-311 | EVQLVQSGSE | VKKPGSSVKVSCQASGSIFS | NYAIN | WVRQAPGQLGEWMG | GIVPIFGTTRFAQKFQG |
| F083-328 | QVQLVQSGAE | VKKPGSSVKLSCKASGGTLR | SYALS | WVRQAPGQLGEWMG | GISAIFNTATYAQNVQG |
| F083-336 | QVQLQQSGAE | VKQPGSSVKVSCKASGGIFR | SNAIS | WVRQAPGQLGEWMG | GIVALFGTANYAQKFQG |
| F083-349 | QVQLVQSGAE | VKKPGSSVKVSCKASGVIFI | KFAIS | WVRQAPGQLGEWMG | GIIPMFGTTNYAQKFQG |
| F083-351 | QVQLVQSGAD | VKKAGSSVKVSCKASGRTFG | NYAIS | WVRQAPGQLGEWMG | GIIPIFGAANYAQKFQG |
| F083-354 | QVQLVQSGSE | VKKPGSSVKVSCKASGGIFN | SYGIS | WVRQAPGQLGEWMG | GILAIFGTTNYAQKFQG |
| F083-366 | QVQLVQSGSE | VKKPGSSVKVSCKASRGIFS | SYAIS | WVRQAPGQLGEWMG | AVIPMFGTLKYAENFQG |
| F083-373 | QVQLVQSGAD | VKKPGSSVRVSCKASGRTFG | NYAIS | WVRQAPGQLGEWMG | GITPIFGAANYAQKFQG |
| FO83-388 | QVQLVQSGAE | VKKPGSSVKVSCKASGDIFN | KRAIT | WVRQAPGQLGEWMG | GITALFATTSYAQKFQD |

| clone | VH FR3 | VH CDR3 | VH FR4 |
|---|---|---|---|
| F080-227 | RVTITADESTSTVYMELSSLRPEDTAVYYCAR | GPNYYFNFFDY | WGQGTLVTVSS |
| F081-007 | RVTITADESSNTVYMDLSRLRSEDTAIYYCAS | HPTYHFDKSGYRFDS | WGQGTLVTVSS |
| F081-008 | RVTITADISTSTAYMELSSLRSEDTAVYYCAR | YQSSDYYNSEYFQH | WGQGTLVTVSS |
| F081-107 | RITISADESTNTVYMELGSLTSEDTAVYYCAT | SGTYYVSYLDS | WGQGTLVTVSS |
| F081-264 | RVIITADASTNIVYMELTSLRSDDTAVYYCAR | SGSYYPDYFQY | WGQGTLVTVSS |
| F081-281 | RLTITSDESTSTAYMELSSLTSEDTAVYYCGR | GPHYYESHLDY | WGQGTLVTVSS |
| F082-117 | RITITADESTNIVHMELSSLRPDDTAVYYCAG | GPHYYVSYFDS | WGQGTLVTVSS |
| F082-220 | RVTITADESTSTNYMELSGLRYEDTAVYYCAR | GPNYYESYFDY | WGQGTLVTVSS |
| F082-237 | RVTISADESSTTVYMELSSLTSEDTAVYYCAR | GNIYYSSYFDQ | WGQGTLVTVSS |
| F082-243 | RVTITADDSTNTVYMELISLRYEDTAVYYCAR | GPNYYENYFDF | WGQGTLVTVSS |
| F082-338 | RVTITADESTNIVSMELSSLRSEDTATYYCAR | SLGYHIQYNGMDV | WGQGTLVTVSS |
| F083-103 | RVTISADLSTRMVYMELSSLRSDDTAFYYCAG | HPTYHYGSAMDY | WGQGTLVTVSS |
| F083-115 | RVTITADDSTNTVYMELTSLRYEDTAVYYCAR | GPNYYESYLDF | WGQGTLVTVSS |
| F083-249 | RVTITADESTRTGFMELSSLRSGDTAVYYCAR | SPTYYPGALEM | WGQGTLVTVSS |
| F083-301 | RVTIAADQSTNTVFMELSSLTSEDTAVYFCAR | SRGYSFGYGTDYFDY | WGQGTLVTVSS |
| F083-302 | RVTITAGELGSTAYMEVRSLTFEDTAVYYCAR | HSGYHLIGYFDS | WGQGTLVTVSS |
| F083-305 | RVTITADESRSTAYMELNNLISEDTAVYYCAR | APLIYNWYFDL | WGQGTLVTVSS |
| F083-307 | RVTISADISTRTAYMELSSLRSEDTAIYYCAR | SPTYYFGSAMEY | WGQGTLVTVSS |
| F083-308 | RVTITADESTSIVYMELSTLRSEDTAVYYCAR | GPNYFESYFDN | WGQGTLVTVSS |
| F083-311 | RVTITADESTSTVYMELGSLTSEDTAVYYCAI | SGIYYVSYFDS | WGQGTLVTVSS |
| F083-311 | RLTITADEPRSTAYMELNNLISEDTAVYYCAR | APLIYNWYYDL | WGQGTLVTVSS |
| F083-328 | RVTITADESTNIFYLEVSSLRSDDTAVYYCAT | SGTYYFSFFDY | WGQGTLVTVSS |
| F083-336 | RVTITADESTSTVYMELSSLRSEDTARYYCAR | NSGYHISGFYLDY | WGQGTLVTVSS |
| F083-349 | RVTITADGSTNTVYMELSSLTSEDTAVYYCAK | EEGYYYGSGPLDS | WGQGTLVTVSS |
| F083-351 | RVTISADLSTRMVYMDLSSLRSDDTAFYYCAG | HPMYHYGSAMDY | WGQGTLVTVSS |
| F083-354 | RVTITADDSSKTVYMELSSLTSEDTAVYYCAR | GSTYYSSYFDQ | WGQGTLVTVSS |
| F083-366 | RITISADKSMSAAYMELSSLRSEDTAVYYCAR | NYYGSGTYFNDAFDI | WGQGTLVTVSS |
| F083-373 | RVIISADLSTTMVYMDLSSLRSDDTAFYYCAG | HPTYYYGSPMDY | WGQGTLVTVSS |
| F083-388 | RVTINWDESTTTAFMELSSLRFEDTAVYYCAR | GPNYYETYLDN | WGQGTLVTVSS |

| Clone | VL FR1 | VL CDR1 | VL FR2 | VL CDR2 |
|---|---|---|---|---|
| F080-227 | DIVMTQSPSSLSASVGDRLDLTC | RASQDIANSLA | WYKQRPGKAPELLVF | GASRLEG |
| F081-007 | QSVLTQPPSVSAAPGQMVTISC | SGSSSNIGNNYVS | WYQQLPGTAPKLLIY | DNNKRPS |
| F081-008 | EIVLTQSPGTLSLSLGERVTLSC | RASQNVGINYLA | WYQQKPGQAPRLLIY | GASSRAT |
| F081-107 | DVVMTQSPGTLSLSPGERATLSC | RASQSVSSSYLA | WYQQKPGQAPRLLIY | GASSRAT |
| F081-264 | EIVLTQSPGTLSLSPGERATLSC | RISQSVSSSYLA | WYQQKPGQAPRLLIH | GASSRAT |
| F081-281 | EIVLTQSPGTLSLSPGERATLSC | RASQGMSSSFLA | WYQQKPGQPPRLLIS | GASTRAT |
| F082-117 | DIQMTQSPATLSLSPGERATLSC | RASQSVRSYLA | WYQQKPGQAPRLLIY | DASNRAT |
| F082-220 | DIVMTQSPPSISASVGDRVTLTC | RASQGISNYLA | WYQQKPGKAPKLLIF | GASTLHS |
| F082-237 | QSVLTQPPPSVSVAPGQTATLTC | GGDKIGTGPVH | WYQQMPGRAPVLLIY | ASTHRPS |
| F082-243 | EIVLTQSPSSLSASLRDTVTITC | RTSQAITNYLA | WYQQRPGKAPKLLIY | AASVLQS |
| F082-338 | DVVMTQSTSSVSASVGDRVTITC | RASQGISSWLA | WYQQKPGKAPKLLIY | AASSLQS |
| F083-103 | QSVLTQPPSVSAAPGQKVTITCS | GSSSNIGNNYVS | WYQQLPGTAPLLLIS | EGSNRPS |
| F083-115 | EIVLTQSPSTLSASVGDRVTITC | RASQSISSWLA | WYQQKPGKAPKLLIY | KASSLES |
| F083-249 | DVVMTQSPSLLSASVGDRVTITC | RASQAIANFVS | WFQQKPGEAPRSLIH | AASTLQG |
| F083-301 | DIVMTQSPSTLSASVGDRVTITC | RASQSISSWLA | WYQQAPGKAPKLLIY | EASKLQS |
| F083-302 | QSVLTQPPSVSAAPGQKVTISC | SGTTSNIGNNYVS | WYQQLPGAAPKLLIY | DNNKRPS |
| F083-305 | EIVLTQSPSFVSASVGDRVSITC | RASQGISSWLA | WYQQKPGKAPRLLIY | GASNLQS |
| F083-307 | SYELTQPPSVSVAPGQTATITC | GGDRIGIGPVH | WYQQRPGRAPVLIIY | GSTHRPS |
| F083-308 | EIVLTQSPATLSLSPGERATLSC | RASQTISSYLA | WYQQKPGQAPRLLIY | GASNRAT |
| F083-311 | DIVMTQSPGTLTLSPGERATLSC | RASQSLYSSHLA | WYQQKPGQPPRLLIY | GASTRAP |
| F083-318 | EIVLTQSPSSLSASVGDRVTITC | RASQGISNYLA | WYQQKPGQPPRLLIY | AASTLQS |
| F083-328 | DTVLTQSPSTLSASVGDRVTITC | RASQSIGSLMA | WYQQQPGKVPKLLIY | RASNLET |
| F083-336 | EIVLTQSPSSLSASVGDRVITIC | RASQGISNYLA | WYQQQPGKPPKLLIY | TASTLLS |
| F083-349 | DIVMTQSPDFLSVSLGERVTINC | KSSQTVLNRSNNKNYLA | WYQKKPGQPPRLLLY | WSSTRES |
| F083-351 | QSVLTQPPSASGTPGQRVSISC | FGSRSNVGSSVN | WYQQLPGRAPFILIQ | RNDQRPS |
| F083-354 | DIQMTQSPSSLSASVGDRNTITC | RASQGIRNSLA | WYQQKPGQVPKLLIY | SATTLRS |
| F083-366 | QSVLTQPPSGSGAPGQRVTISC | TGGNSNIGAGYDVN | GYQQLPGKAPKLLIY | GNNNRPS |
| F083-373 | EIVLTQSESSISASVGDRVTITC | RASQSIATYLN | WYQQKPGKAPNLLIY | AASNLQS |
| F083-389 | EIVLTQSPTILSASVGDRVTITC | RASQSISRWLA | WYQQKPGKVPKLLIY | MASTLES |

| Clone | VL FR3 | VL CDR3 | VL FR4 |
|---|---|---|---|
| F080-227 | GVPSRFSGTRAGTDFTLTISSLQPEDLGTYFC | QQYFSFPRT | FGQGTTLDLR |
| F081-007 | GIPDRFSGSKSGTSATLGITGLQTGDEADYYC | GTWDSSLSAGV | FGGGTKLTVLG |
| F081-008 | GVPDRFSGSGSGTDFTLTISRLAPEDFAVYYC | QQYSSSPLT | FGGGTLLEIK |
| F081-107 | GIPDRFSGSGSGTDFTITISRLEPEDFAVYYC | QQYGSSPLT | FGPGTKVDIK |
| F081-264 | GIPDRFSGSGSGTDFTISISRLEPEDFAVYYC | QQYGSSPRT | FGQGTKVEIK |
| F081-281 | GIPDRFSGSGSGTDFTLTINRLEPEDFAVYYC | QQYGSSPRT | FGQGTKVEIK |
| F082-117 | GIPARFSGSGSGTEFTLTISRLEPEDFAVYYC | QQRSNWLFT | FGPGTKVDFK |
| F082-220 | GVSSRFSGSGSGPDFLTTISSLQPEDAATYYC | QKYHSAPLT | FGGGTKVEMK |
| F082-237 | RIPERFSGSKSDUTATISISGVGVGDEADYYC | QMWDSSSVHPIV | FGGGTKLTVLS |
| F082-243 | GVPSRFSGSGSGIDIILTISSIQPEDFATYYC | QKYDSAPYT | FGQGTKLEIK |
| F082-338 | GVPSRFSGSGSGTDFTLTISSLQPEDFATYYC | QQANSFPIT | FGQGIRLEIK |
| F083-103 | GIPDRFSGSKSGTSATLGITGLQTGDEATYYC | ATWDDSLSVVL | FGGGTRLTVLS |
| F083-115 | GVPSRFSGSGSGTEFTLTISSLPQDDFATYYC | QQYNSYPRT | FGQGTKVEIK |
| F083-249 | GVPPRFSGSGYGAVTILTITNLQPEDFATYFC | QQYNSLPFT | FGPGTDVEVR |
| F083-301 | GVPSRFSATGSGTEFTLTIASLQPEDFAIYYC | QHYEAYPFS | FGPGTKLDVK |
| F083-302 | GIPDRFSGSKSDTSATLGITGLQTGDEADYYC | GTWVSSLSVWV | FGGGTKLTVLG |
| F083-305 | GVPSRFSGSGSGTDYTLTINSLQPEDFATYYC | QQARTFPVT | FGQGTKLEIK |
| F083-307 | GIPERFSGSKSVNTATLSISGVGVGDEADYYC | QVWDTSTAQPT | VFGGGTKLTVLS |
| F083-308 | GIPARFSGSGSGTDFTITINSLEPEDFAVYYC | QQRSSWPPIT | FGQGTRLEIK |
| F083-311 | GTPDRFSGSGSGTDFTLTITALDPEDSAVYYC | QQYGSSPIT | FGQGTRLEIK |
| F083-318 | GVPSRFSGSGSGTDFTLTISSIQPEDVATYYC | QKYNSAPHT | FGPGTKVDIK |
| F083-328 | GVPSRFTGSGSGTEFALTISSLQPDDVGTYYC | QHFKIFSRT | FGQGTKVEIK |
| F083-336 | GVPSRFSGGGSGTDYTLTISSLQPEUVATYYC | QEYKSVPLT | FGPGTKVEIK |
| F083-349 | GVPDRFSGSGSETDFALTITNLQAEDLAVYFC | QQFYDMPVT | FGGGTKLEIK |
| F083-351 | GVPDRFSGSHSGTSASLAISGLQSDDESTYYC | AAWDDSVKSVV | FGGGTQLTVLG |
| F083-354 | GVPYRYSGSGSGTDFTLTISGLOPEDVGTYFC | HRYDSAPLT | FGGGTRVALK |
| F083-366 | GVPDRFSGSKSGISASLAIIGLQVEDEAHYYC | QSYDNGLSGSV | FGGGIQLIVLG |
| F083-373 | GVPSRFSGSGSGTDFTLTISSLQPEDFATYVC | LQSYSATLT | FGGGTKVEIK |
| F083-389 | GVPSRFSGSGSGSEFTLTISSIQPGDFATYYC | QQYNSYERT | FGQGTKVEINVK |

If an epitope recognized by the neutralizing antibody of the present invention were clarified, a peptide comprising the amino acid sequence of the epitope (an amino acid sequence having antigenicity) would be useful as a vaccine for influenza virus, and also, a nucleic acid (gene) comprising a nucleotide sequence encoding a peptide having the antigenicity would be useful as a reagent used for influenza tests or a reagent kit for the tests. An immunologically reactive epitope can be determined by a known method. Examples of such a method of specifying an immunologically reactive epitope include: 1) a method of examining the reactivity of a limited decomposition product prepared by subjecting hemagglutinin to an enzymatic treatment or a chemical treatment with an IgG-type antibody of the neutralizing antibody obtained by the present invention; and 2) a method of examining the reactivity of an overlapping peptide synthesized with reference to amino acid sequence database with an IgG-type antibody of the neutralizing antibody obtained by the present invention.

### (Production of the Antibody of the Present Invention)

An H chain or L chain expression vector is produced, and it is then allowed to express in a host cell. Thereafter, a supernatant containing the secreted antibody is recovered and is then purified, so that an antibody can be obtained.

Specifically, DNA encoding VH is ligated to another DNA molecule encoding a heavy chain constant region (CH1, CH2 and CH3), so as to obtain a full-length heavy chain gene. The sequence of a human heavy chain constant region gene is known in the present technical field (for example, Kabat, E. A. et al., (1991) Sequences of Proteins of Immunological Interest, 5^{th} edition, U. S. Department of Health and Human Services, NIH Publication No. 91-3242), and a DNA fragment including such a region can be obtained by standard PCR amplification. The heavy chain constant region may be the constant region of IgG1, IgG2, IgG3, IgG4, IgA, IgE, IgM or IgD. The most preferred constant region is that of IgG1 or IgG2. The constant region sequence of IgG1 may include any given various alleles or allotypes known to be generated among different individuals, such as Gm (1), Gm (2), Gm (3) or Gm (17). These allotypes correspond to a substitution of amino acids naturally-occurring in the constant region of IgG1.

DNA encoding VL is ligated to another DNA molecule encoding the light chain constant region CL, so as to obtain a full-length L chain gene (and a Fab light chain gene). The sequence of a human light chain constant region gene is known in the present technical field (for example, Kabat, E. A. et al., (1991) Sequences of Proteins of Immunological Interest, 5^{th} edition, U. S. Department of Health and Human Services, NIH Publication No. 91-3242), and a DNA fragment including such a region can be obtained by standard PCR amplification. The light chain constant region may be the constant region of κ or λ. The κ constant region may include any given various alleles known to be generated among different individuals, such as Inv (1), Inv (2) or Inv (3). The λ constant region may be derived from any one of the three λ genes.

The thus obtained DNA encoding an H chain or L chain is inserted into a vector to produce an expression vector, and the produced expression vector is then allowed to express in a host cell. Thereafter, a supernatant containing the secreted antibody is recovered and purified, so that an antibody can be obtained. Examples of the expression vector include a plasmid, retrovirus, adenovirus, adeno-associated virus (AAV), plant viruses such as cauliflower mosaic virus or tobacco mosaic virus, a cosmid, YAC, and EBV-derived episome. An expression vector and an expression regulatory sequence are selected, so that they are suitable for a host cell used for expression. An antibody light chain gene and an antibody heavy chain gene can be inserted into different vectors, or the two genes can also be inserted into a single expression vector. An antibody gene is inserted into an expression vector by a standard method (for example, ligation of a complementary restriction site on an antibody gene fragment to a vector, or blunt-ended ligation applied when no restriction sites are present).

A favorable vector encodes a functionally completed human CH or CL immunoglobulin sequence having a suitable restriction site, which has been produced by an engineering approach such that any given VH or VL sequence can be easily inserted and then expressed as described above. In such a vector, splicing generally takes place between a splice donor site in the inserted J region and a splice acceptor site preceding a human C domain, or such splicing also takes place in a splice region existing in a human CH exon. Polyadenylation and transcription termination take place in a natural chromosomal site downstream of a coding region. A recombinant expression vector can also encode a host cell-derived signal peptide that promotes the secretion of an antibody chain. An antibody chain gene can be cloned into a vector, such that a signal peptide can be ligated in-frame to the amino terminus of an immunoglobulin chain. The signal peptide may be either an immunoglobulin signal peptide or a heterogeneous signal peptide (namely, it may be a non-immunoglobulin protein-derived signal peptide).

An expression vector used for the antibody of the present invention may also have sequences such as a sequence for regulating replication of the vector in a host cell (e.g. a replication origin) or a selective marker gene sequence, in addition to an antibody gene and a regulatory sequence. The selective marker gene promotes selection of a host cell into which a vector has been introduced. For instance, the selective marker generally imparts resistance to drugs such as G418, hygromycin or methotrexate to a host cell into which the vector has been introduced. Preferred selective marker genes include a dihydrofolate reductase (DHFR) gene (used in methotrexate selection/amplification as a dhfr-host cell), a neomycin phosphotransferase gene (used in G418 selection), and a glutamate synthase gene.

A host cell is transformed with an antibody gene expression vector produced by the above-described method. Any type of cell may be used as a host cell, as long as it can produce the antibody of the present invention. Examples of such a host cell include bacteria, yeast, animal cells, insect cells, and plant cells. Among these cells, animal cells are preferable. Examples of the animal cells include Chinese hamster ovary cells CHO/dhfr(−) and CHO/DG44, monkey-derived cells COS (A. Wright & S. L. Morrison, J. Immunol. 160, 3393-3402 (1998)), and SP2/O cells (mouse myeloma) (K. Motmans et al., Eur. J. Cancer Prev. 5, 512-5199 (1996), R. P. Junghans et al., Cancer Res. 50, 1495-1502 (1990)). For transformation, a lipofectin method (R. W. Malone et al., Proc. Natl. Acad. Sci. USA 86, 6007 (1989), P. L. Felgner et al., Proc. Natl. Acad. Sci. USA 84, 7413 (1987)), an electroporation method, a calcium phosphate method (F. L. Graham & A. J. van der Eb, Virology 52,456-467 (1973)), a DEAE-Dextran method, and the like are preferably applied.

A transformant is cultured, and an antibody is then separated from the cells of the transformant or a culture medium thereof. For separation/purification of the antibody, methods such as centrifugation, ammonium sulfate fractionation, salting-out, ultrafiltration, affinity chromatography, ion exchange chromatography and gel filtration chromatography can be used by appropriately combining them.

### (Antibody Fragments)

An antibody fragment can be produced based on the antibody of the present invention, or based on the sequence information of a gene encoding the antibody of the present invention. Examples of the antibody fragment include Fab, Fab′, F(ab′)₂, scFv, and dsFv antibodies.

Fab is obtained by digesting IgG by papain in the presence of cysteine. It is an antibody fragment with a molecular weight of approximately 50,000, which is constituted with L chain and H chain variable regions, and an H chain fragment consisting of a CH1 domain and a portion of a hinge region. In the present invention, the above-described antibody can be obtained by papain digestion. In addition, Fab can also be prepared by incorporating DNA encoding a portion of the H chain and the L chain of the above-described antibody into a suitable vector, then performing transformation with the resulting vector, and then obtaining it from the transformant.

Fab′ is an antibody fragment with a molecular weight of approximately 50,000, which is obtained by cleaving a disulfide bond between the H chains of the below-mentioned F(ab′)₂. In the present invention, Fab′ can be obtained by digesting the above-described antibody by pepsin, and then cleaving a disulfide bond with a reducing agent. In addition, as with Fab, Fab′ can also be prepared by genetic engineering using DNA encoding the Fab′.

F(ab′)₂ is obtained by digesting IgG by pepsin, and is an antibody fragment with a molecular weight of approximately 100,000, which is obtained by binding, via a disulfide bond, one fragment (Fab′) constituted with L chain and H chain variable regions and an H chain fragment consisting of a CH1 domain and a portion of a hinge region, to the other fragment (Fab′). In the present invention, F(ab′)₂ can be obtained by digesting the above-described antibody by pepsin. In addition, as with Fab, F(ab′)₂ can also be prepared by genetic engineering using DNA encoding the F(ab′)₂.

scFv is an antibody fragment obtained by ligating the C-terminus of one chain of Fv consisting of an H chain variable region and an L chain variable region to the N-terminus of the other chain thereof, using a suitable peptide linker, so as to form a single chain. (GGGGS)₃ having high flexibility can be used, for example, as such a peptide linker. For instance, DNA encoding the H chain variable region and L chain variable region of the above-described antibody and DNA encoding a peptide linker are used to construct DNA encoding a scFv antibody, and the thus constructed DNA is then incorporated into a suitable vector. Thereafter, scFv can be prepared from a transformant obtained by transformation with the aforementioned vector

dsFv is a Fv fragment obtained by introducing a Cys residue into a suitable site in each of an H chain variable region and an L chain variable region, and then stabilizing the H chain variable region and the L chain variable region by a disulfide bond. The site in each chain, into which the Cys residue is to be introduced, can be determined based on a conformation predicted by molecular modeling. In the present invention, for example, a conformation is predicted from the amino acid sequences of the H chain variable region and L chain variable region of the above-described antibody, and DNA encoding each of the H chain variable region and the L chain variable region, into which a mutation has been introduced based on such prediction, is then constructed. The thus constructed DNA is incorporated into a suitable vector. Thereafter, dsFv can be then prepared from a transformant obtained by transformation with the aforementioned vector.

Further, it is also possible to ligate scFv antibody or dsFv antibody using a suitable linker, or to fuse an antibody fragment with streptavidin, so as to multimerize the antibody fragment.

### (Pharmaceutical Composition)

Since the neutralizing antibody of the present invention is capable of neutralizing influenza viruses of all hemagglutinin subtypes over the framework of groups (which is not an antibody capable of neutralizing influenza viruses beyond the framework of groups), it can be an effective preventive and/or therapeutic means, not only for seasonal influenza caused by antigenic drift, but also for pandemic influenza caused by antigenic shift. With regard to the range of the present neutralizing antibody (which may also be effective for antigenic shift, if the antigenic shift is a shift in Group 1), passive immunization can be carried out on influenza viruses of subtypes in Group 1 by administering the neutralizing antibody, and therapeutic effects on patients who have developed influenza as a result of infection with any given influenza virus, and preventive effects on subjects who have a risk of the onset of influenza or infection with influenza virus can be anticipated. Moreover, since the neutralizing antibody of the present invention has originally existed in a human body, it is considered that the present neutralizing antibody does not have a risk of side effects.

The neutralizing antibody of the present invention can be used as a passive immunotherapeutic agent against influenza, by itself, or after it has been mixed with a pharmaceutically acceptable carrier to prepare a pharmaceutical composition.

Herein, as pharmaceutically acceptable carriers, various types of organic or inorganic carrier substances, which have been commonly used as materials for formulations, are used. These pharmaceutically acceptable carriers are mixed as excipients, solvents (dispersers), solubilizers, suspending agents, stabilizers, isotonization agents, buffers, pH adjusters, soothing agents, and the like, with the neutralizing antibody of the present invention. In addition, as necessary, formulation additives, such as preservatives and antioxidants, can also be used.

Preferred examples of the excipient include lactose, saccharose, D-mannitol, D-sorbitol, starch, pregelatinized starch, dextrin, crystalline cellulose, low-substituted hydroxypropyl cellulose, sodium carboxymethyl cellulose, gum Arabic, Pullulan, light anhydrous silicic acid, synthetic aluminum silicate, and magnesium aluminometasilicate.

Preferred examples of the solvent include water for injection, a normal saline, a Ringer solution, alcohol, propylene glycol, polyethylene glycol, sesame oil, corn oil, olive oil, and cottonseed oil.

Preferred examples of the solubilizer include polyethylene glycol, propylene glycol, D-mannitol, trehalose, benzyl benzoate, ethanol, tris-aminomethane, cholesterol, triethanolamine, sodium carbonate, sodium citrate, sodium salicylate, and sodium acetate.

Preferred examples of the suspending agent include: surfactants such as stearyl triethanolamine, sodium lauryl sulfate, laurylaminopropionic acid, lecithin, benzalkonium chloride, benzethonium chloride and glyceryl monostearate; hydrophilic polymers such as polyvinyl alcohol, polyvinyl pyrrolidone, sodium carboxymethyl cellulose, methyl cellulose, hydroxymethyl cellulose, hydroxyethyl cellulose and hydroxypropyl cellulose; and polysorbates and polyoxyethylene hardened castor oil.

Preferred examples of the stabilizer include human serum albumin (HSA), sodium pyrosulfite, Rongalit, and sodium hydrogen metabisulfite.

Preferred examples of the isotonization agent include sodium chloride, glycerin, D-mannitol, D-sorbitol, and glucose.

Preferred examples of the buffer include buffer solutions such as phosphate, acetate, carbonate, and citrate.

Preferred examples of the pH adjuster include acids and bases, such as hydrochloric acid and sodium hydroxide.

A preferred example of the soothing agent is benzyl alcohol.

Preferred examples of the preservative include p-hydroxybenzoate esters, chlorobutanol, benzyl alcohol, phenethyl alcohol, dehydroacetic acid, and sorbic acid.

Preferred examples of the antioxidant include sulfite and ascorbate.

Examples of the dosage form of the above-described pharmaceutical composition include injections (e.g., a subcutaneous injection, an intravenous injection, an intramuscular injection, an intraperitoneal injection, an intra-arterial injection, etc.) and injection-type formulations such as drops.

The pharmaceutical composition can be produced by a method commonly used in the technical field of formulations, for example, a method described in Japanese Pharmacopoeia. Hereafter, specific methods for producing formulations will be described in detail. The content of an antibody in a pharmaceutical composition is different depending on the dosage form, the applied dose, etc. It is, for example, approximately 0.1% to 100% by weight.

For example, an injection is produced by dissolving, suspending or emulsifying an antibody, together with a disperser (e.g., polysorbate 80, polyoxyethylene hardened castor oil 60, polyethylene glycol, carboxymethyl cellulose, sodium alginate, etc.), a preservative (e.g., methylparaben, propylparaben, benzyl alcohol, chlorobutanol, phenol, etc.), an isotonization agent (e.g., sodium chloride, glycerin, D-mannitol, D-sorbitol, glucose, etc.) and the like, in an aqueous solvent (e.g., distilled water, normal saline, Ringer's solution, etc.) or in an oily solvent (e.g., vegetable oil such as olive oil, sesame oil, cottonseed oil or corn oil, propylene glycol, etc.). At this time, additives such as a solubilizer (e.g., sodium salicylate, sodium acetate, etc.), a stabilizer (e.g., human blood albumin, etc.), a soothing agent (e.g., benzyl alcohol, etc.) may be used, as desired. Such an injection is subjected to a sterilization treatment such as filtration sterilization using a membrane filter or the like, as necessary, and thereafter, it is generally filled into a suitable vessel such as an ampule.

Regarding such an injection, the aforementioned liquid agent is processed into powders according to vacuum drying or the like, and the particles can be then used by being dissolved (dispersed) before use. Examples of the vacuum drying method include a freeze-drying method and a method using a speed-back concentrator (SAVANT). When such freeze-drying is carried out, it is preferable that a sample that has been cooled to −10° C. or lower be used, and that the sample be freeze-dried in a flask in a laboratory, industrially using a tray, or in a vial. When such a speed-back concentrator is used, freeze-drying is carried out at approximately 0° C. to 30° C., and at a degree of vacuum of approximately 20 mmHg or less, and preferably approximately 10 mmHg or less. It is preferable that a buffer such as phosphate be added to a liquid agent to be dried, and that the pH be set at approximately 3 to 10. The powdery formulation obtained by vacuum drying is stable for a long period of time, and it can be used as an injection by being dissolved in water for injection, normal saline, Ringer's solution, etc., or being dispersed in olive oil, sesame oil, cottonseed oil, corn oil, propylene glycol, etc., before use.

Other therapeutic drugs can also be used in combination with the above-described antibody, as necessary. Examples of such a therapeutic drug include Tamiflu, Relenza, and Amantadine.

Otherwise, other therapeutic drugs can also be bound to the above-described antibody, as necessary. The antibody transports a drug to a site at which influenza virus is present, or a periphery thereof, and it also inhibits invasion of the virus into cells. On the other hand, the drug kills the virus, or it treats, alleviates or ameliorates the symptoms of influenza. The drug that is used herein includes all drugs, which are used as or are to be used as therapeutic drugs for influenza. Such a drug is, for example, a synthetic or natural, low-molecular-weight or high-molecular-weight, protein or non-protein, or nucleic acid or nucleotide substance. The binding of the antibody to the drug is preferably carried out via a linker. The linker comprises, for example, a substituted or non-substituted aliphatic alkylene chain, and at both ends thereof, it comprises a group capable of binding to a functional group of the antibody or drug, such as an N-hydroxysuccinimide group, an ester group, a thiol group, an imidocarbonate group, or an aldehyde group (Kotai Kogaku Nyumon (Antibody Engineering Manual), Chijinshokan Co., Ltd., 1994).

As necessary, in order to facilitate the transport of a pharmaceutical drug into a cell, the drug can also be encapsulated into a liposome. Examples of a preferred liposome include a positively charged liposome, positively charged cholesterol, and a transmembrane peptide binding liposome (Mamoru NAKANISHI et al., Tanpakushitsu Kakusan Koso (Protein, Nucleic acid and Enzyme 44: 1590-1596 (1999), Shiro NIKI, Kagaku to Seibutsu (Chemistry and Organisms) 43: 649-653 (2005), Clinical Cancer research 59: 4325-4333 (1999), etc.).

The neutralizing antibody of the present invention is administered via parenteral administration, such as intravenous administration, intraperitoneal administration, intramuscular administration, subcutaneous administration, or transdermal administration.

The amount of the active ingredient of the antibody is 100 to 2,500 μg/ml per dose, or it is 1.0 to 10 mg per kg of the body weight of an adult human patient. However, the amount of the active ingredient is not limited to thereto.

The number of administrations is, for example, once 1 to 2 weeks for one to several administrations, or once 2 to 3 weeks for approximately 2 months.

The neutralizing antibody of the present invention is not only used for prevention and/or treatment of influenza for humans, but it is also administered to avian species such as a chicken, or to non-human mammals such as a swine or a horse, so that it can also be used for prevention and/or treatment of influenza for such animals. Thereby, the risk of infection of humans with influenza can be reduced before it happens. Even in a case where the present neutralizing antibody is applied to these animals, the same formulation method as described above can be applied.

Hereinafter, the present invention will be described more in detail in the following examples. However, these examples are not intended to limit the scope of the present invention.

### EXAMPLES

### Materials and Methods

### Materials

### <Antigen Virus for Screening>

The following influenza viruses were used in the present study: A/H1N1pdm: A/California/7/2009pdm (Cal09), and A/Brisbane/59/2007 (Bri07).

The terms in the parentheses indicate abbreviations of virus strains.

### Characterization (for ELISA, HI Activity, and Neutralizing Activity)

The following influenza viruses were used in the present study: A/H1N1pdm: A/California/7/2009pdm (Cal09), A/Suita/1/2009pdm (Sui09); A/H1N1: A/New Caledonia/20/1999 (NC99), A/Solomon Islands/3/2006 (SI06), A/Brisbane/59/2007 (Bri07). A/H3N2: A/Panama/2007/1999. A/H5N1: A/Indonesia/5/2005/PR8-IBCDC-RG2. The terms in the parentheses indicate abbreviations of virus strains.

### <Construction of Phage Antibody Library>

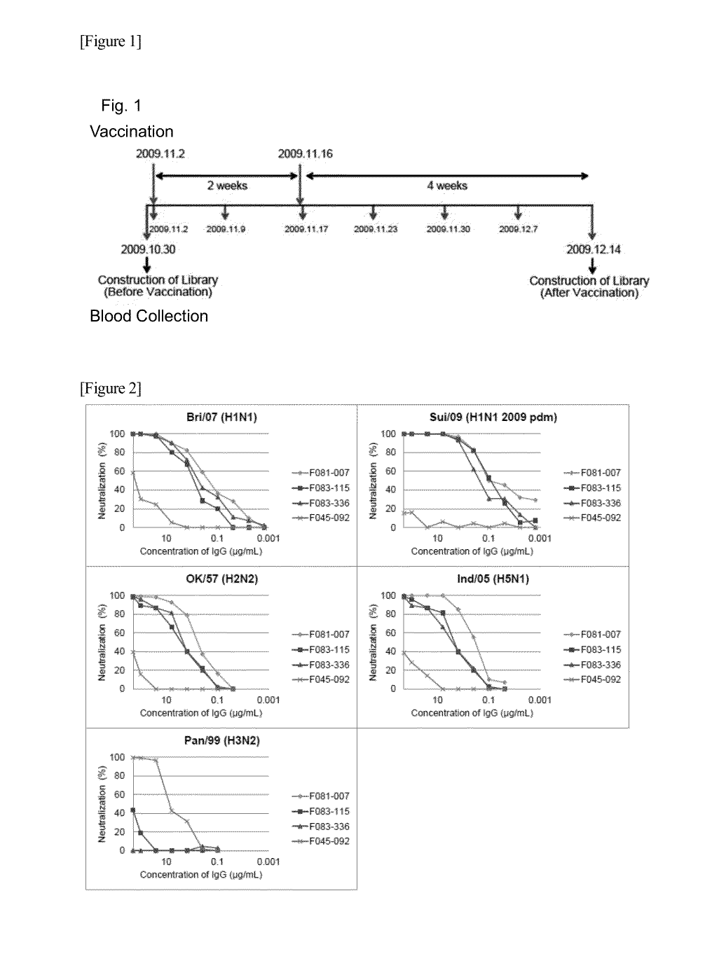

The schedule of vaccination and blood collection is shown in FIG. 1.

In 2009, a donor bone in 1947 was inoculated with the pandemic vaccine strain A/California/7/2009 twice (November 2 and 16, 2009). Blood collection from the donor was carried out, twice before the vaccinations (October 30 and November 2), once after the first vaccination (November 9), and five times after the second vaccination (November 17, 23 and 30, and December 7 and 14). From the blood collected on October 30 (before the vaccinations) and on December 14 (after the vaccinations), an enormous phage antibody library was prepared.

Such a phage antibody library was constructed according to the previously reported method (Okada J, et al., 2010. Virology 397: 322-330). The monocytes of the donor born in 1947 before and after the vaccinations were recovered from 3 L of blood according to apheresis. The cells comprised 8.0×10⁸ B lymphocytes before the vaccinations and 1.2×10⁹ B lymphocytes after the vaccinations. The enormous antibody library was constructed by a phage display method, as previously reported (Marks J D, et al., 1991. J. Mol. Biol. 222: 581-597). With regard to the size of the library, from the blood collected before the vaccinations, the heavy chain consisted of 1.6×10⁹ clones, the light chain consisted of 2.0×10⁹ clones, and Fab consisted of 1.4×10¹⁰ clones. From the blood collected after the vaccinations, the heavy chain consisted of 3.2×10⁹ clones, the light chain consisted of 1.3×10⁹ clones, and Fab consisted of 2.6×10¹⁰ clones.

### Antibody-Obtaining Method

### <Screening of Library>

Clones binding to viral particles were selected by a panning method, as previously reported (Iba Y, et al., 1997. Gene 194: 35-46). The viral particles H1N1 of the strain Cal09 or Bri07, which had been treated with formalin, were used as antigens in a screening operation. After two or three times of panning operations, Escherichia coli (DH12S) was infected with the eluted phage, and it was then cultured in an LB plate containing 100 μg/ml ampicillin and 0.2% glucose. Thereafter, Escherichia coli colonies containing phagemid were isolated, and they were then cultured at 30° C. overnight in a 2×YT medium containing 100 μg/ml ampicillin, 0.05% glucose and 1 mM isopropyl-β-D-thiogalactopyranoside (IPTG). During the culture of the Escherichia coli, a Fab-cp3-type antibody was secreted into the medium (Morino K, et al., 2001. J. Immunol. Meth 257: 175-184). ELISA was carried out using the culture supernatant containing the Fab-cp3-type antibody, and the binding activity of the antibody to the virus strains H1N1 and H3N2 used in the screening was measured. Clones only binding to the virus strain H1 were selected and were subjected to a further analysis.

### Antibody Evaluation Method

### <ELISA>

A 96-well maxisorp immunoplate (Nunc) was coated with viral particles that had been treated with formalin, and the Fab-cp3-type antibody contained in the culture supernatant of Escherichia coli was then added to each well. The well was incubated with a rabbit anti-cp3 antibody (MBL), and it was further incubated with peroxidase-conjugated goat anti-rabbit IgG (H+L chain; MBL). A HRP substrate (OPD, Wako Pure Chemical Industries, Ltd.) was added to each well, so that a color was developed. The peroxidase reaction was terminated by addition of sulfuric acid, and the absorbance of the sample at a wavelength of 492 nm was then measured.

### <Sequence Analysis>

The nucleotide sequence of the VH fragment of the isolated antibody clone was determined by employing GenomeLab Dye Terminator Cycle Sequencing—Quick Start Kit (Beckman Coulter) and CEQ2000 DNA Analysis System (Beckman Coulter). As a VH sequence primer, T7ETZ (5′-TAATACGACTCACTATAGGG-3′) was used.

### <Western Blotting>

A vaccine strain, which had been inactivated with formalin, was subjected to SDS-PAGE under non-reduction conditions, and it was then transcribed on Immobilon-P membrane (Millipore). The membrane was incubated with the Fab-cp3-type antibody contained in the culture supernatant of Escherichia coli. The membrane was incubated with rabbit anti-cp3 Ab (MBL), and was further incubated with peroxidase-conjugated goat anti-rabbit IgG (H+L chain; MBL). A band was detected using ECL Plus Western blotting Detection Regents (GE Healthcare) and Chemical-luminescence imaging system (Light Capture; ATTO).

### <Measurement of Virus-Neutralizing Activity>

A focus reduction neutralization assay was carried out (Okuno Y, et al., 1990. J. Clin. Microbiol. 28: 1308-13131). A Fab-PP-type antibody (200 or 500 μg/ml) was mixed with an equal amount of influenza virus (100 FFU), and the obtained mixture was then added to MDCK cells, which had been allowed to grow on a 96-well plate. After completion of incubation, the cells were washed with serum-free MEM, and were then cultured at 37° C. for 15 hours in MEM containing 0.4% BSA. Thereafter, the cultured cells were fixed with ethanol, and were then allowed to react with a PAP (peroxidase and anti-peroxidase) complex. The number of focuses, which were formed with one or more cells, was counted, and a focus reduction rate was shown as a result.

### <Hemagglutination Inhibition (HI) Assay>

The HI test was carried out by the previously mentioned method Okuno Y, et al., 1990. J. Clin. Microbiol. 28: 1308-13131). The purified 160 μg/ml Fab-PP-type antibody was subjected to stepwise dilution, and it was then incubated with virus of 4 HA units per well. Thereafter, 0.75% Guinea pig serum was added to each well, and it was then incubated at room temperature for 30 to 60 minutes. The lowest concentration (μg/ml) of the Fab-PP-type antibody that inhibited hemagglutination was shown as a result.

### Antibody Production Methods (Fab and IgG)

### <Production of Purified Fab-PP-Type Antibody>

A Fab-cp3-type antibody was converted to a Fab-PP-type antibody (wherein P represents a single Fe-binding domain of protein A) according to a previously reported method (Ito W, et al., 1993. J. Biol. Chem. 268: 20668-20675), and the Fab-PP-type antibody was then purified with IgG Sepharose (GE Healthcare).

### <Production of IgG-Type Antibody>

### (1) Production of Plasmid Expressing IgG Antibody

The gene of a phage antibody (scFv) is disposed in the order of VH-VL, and VH and VL have an scFv structure in which they are connected with each other by the following linker.

Linker (SEQ ID NO: 291):
Gly Gly Gly Gly Ser Gly Gly Gly Gly Ser Gly Gly
Gly Gly Ser

On the other hand, the gene of a phage antibody (Fab) is disposed in the order of VH-CH1-His and VL-CL-PIE, and the aforementioned connecting linker is not present.

First, a human germline gene, which was assumed to be used in VH and VL, was searched on IMGT (*).

(*) IMGT: http://www.imgt.org

Using the search results of IMGT as references, the phage antibody was converted to an IgG antibody. A VH sequence was connected with the constant region of human IgG1. Regarding VL, a gene, a deficient portion of which was connected with the closest germline gene, was produced. An H chain gene and an L chain gene, in which NheI was added to the 5′-terminal side and EcoRI was added to the 3′-terminal side, were totally synthesized by GenScript. The synthesized heavy chain and light chain genes were each incorporated into different expression vectors. That is to say, the artificially synthesized H chain and L chain genes were each cleaved with NheI and EcoRI, and they were then incorporated into the NheI and EcoRI sites of the expression vector pCAGGS, so as to obtain an antibody H chain expression vector and an antibody L chain expression vector. After construction of these expression vectors, they were allowed to forcibly express in human cells, thereby obtaining antibodies (seethe content of Japanese Patent No. 4870348).

### [Results]

The activities of representative clones are shown in Table 4.

TABLE 4

| | | Number | Library | | | |
|---|---|---|---|---|---|---|
| | | of | Cal09 | | Bri07 | |
| | | isolated | Before | After | Before | After |
| Group | Type | clones | vaccination | vaccination | vaccination | vaccination |
| 1 | 2 | 5 | 1 | | 4 | |
| 2 | 4 | 12 | 1 | 1 | 1 | 9 |
| 36 | 2 | 2 | | 1 | | 1 |
| 49 | 1 | 1 | | | | 1 |
| 47 | 2 | 7 | | | | 7 |
| 48 | 2 | 2 | | | | 2 |
| 3 | 2 | 2 | 1 | | 1 | |
| 35 | 1 | 1 | | 1 | | |
| 39 | 2 | 4 | | 2 | | 2 |
| 54 | 1 | 3 | | | | 3 |
| 37 | 2 | 6 | | 1 | | 5 |
| 41 | 2 | 4 | | | 3 | 1 |
| 52 | 1 | 1 | | | | 1 |
| 40 | 3 | 3 | | | 2 | 1 |
| 42 | 2 | 6 | | | 1 | 5 |
| 55 | 1 | 1 | | | | 1 |
| 56 | 1 | 1 | | | | 1 |
| 57 | 1 | 2 | | | | 2 |
| 58 | 1 | 2 | | | | 2 |
| 80 | 1 | 1 | | | | 1 |
| 59 | 1 | 1 | | | | 1 |
| 51 | 1 | 2 | | | | 2 |
| 62 | 2 | 2 | | | | 2 |
| 63 | 1 | 1 | | | | 1 |
| 38 | 2 | 4 | | 1 | | 3 |
| 43 | 1 | 2 | | | 2 | |
| 64 | 1 | 1 | | | | 1 |
| 65 | 1 | 1 | | | | 1 |
| 44 | 1 | 1 | | | 1 | |

| | | Identity | | | |
|---|---|---|---|---|---|
| | | (%) to | Amino acid | HI activity (μg/ml) | |
| | Germline | germline | sequence of | | Cal09 |
| Group | gene | gene | CDR3 region | Bri07 | pdm |
| 1 | 1-69*01 | 92.2 | GPNYYENFPDY | >160 | >160 |
| 2 | 1-69*01 | 88.6 | GPNYYESYFDY | >160 | >160 |
| 36 | 1-69*01 | 88.6 | GPNYYENYFDF | >160 | >160 |
| 49 | 1-69*01 | 85.2 | GPNYYESYLDF | >160 | >160 |
| 47 | 1-69*01 | 89.8 | GPNYFESYFDN | >160 | >160 |
| 48 | 1-69*01 | 79.5 | GPNYYETYLDN | >160 | >160 |
| 3 | 1-69*01 | 85.6 | GPHYYESHLDY | >160 | >160 |
| 35 | 1-69*01 | 50.7 | GPHYYVSYFDS | >160 | >160 |
| 39 | 1-69*01 | 86.4 | GNTYYSSYFDQ | >160 | >160 |
| 54 | 1-69*01 | 87.5 | GSTYYSSYFDQ | >160 | >160 |
| 37 | 1-69*01 | 85.2 | SGTYYVSYFDS | >160 | >160 |
| 41 | 1-69*01 | 81.8 | SGTYYVSYLDS | >160 | >160 |
| 52 | 1-69*01 | 80.7 | SGTYYVSFFDY | >160 | >160 |
| 40 | 1-69*01 | 84.1 | SGSYYPDYPQY | >160 | >160 |
| 42 | 1-69*01 | 89.8 | SPTYYPGALDM | >160 | >160 |
| 55 | 1-69*01 | 86.4 | APLIYNWYFDL | >160 | >160 |
| 56 | 1-69*01 | 83.0 | APLIYNWYYDL | >160 | >160 |
| 57 | 1-69*01 | 83.0 | HPTYHYGSAMDY | >160 | >160 |
| 58 | 1-69*01 | 89.8 | HPTYYFGSAMEY | >160 | >160 |
| 80 | 1-69*01 | 83.0 | HPTYYYGSFMDY | >160 | >160 |
| 59 | 1-69*01 | 83.0 | HPMYHYGSAMDY | >160 | >160 |
| 51 | 1-69*01 | 80.7 | HSGYNLIGYFDS | >160 | >160 |
| 62 | 1-69*01 | 85.2 | EEGYYYGSGPLDS | >160 | >160 |
| 63 | 1-69*01 | 89.8 | NSGYHISGFYLDY | >160 | >160 |
| 38 | 1-69*01 | 86.4 | SLGYHTQFNGMDY | >160 | >160 |
| 43 | 1-69*01 | 85.2 | HPTYHFDKSGYRFDS | >160 | >160 |
| 64 | 1-69*01 | 87.5 | SRGYSFGYGTDYFDY | >160 | >160 |
| 65 | 1-69*01 | 84.1 | NYYGSGTYFNDAFDI | >160 | >160 |
| 44 | 1-69*02 | 92.3 | YQSSDYYNSSYEQH | >160 | >160 |

| | Binding activity | | | | | |
|---|---|---|---|---|---|---|
| | H1N1 | | | | H3N2 | no |
| Group | NC99 | S106 | Bri07 | Cal09pdm | Pan99 | coating |
| 1 | 3.15 | 3.16 | 3.10 | 2.95 | 0.14 | 0.15 |
| 2 | 3.37 | 3.37 | 3.15 | 2.72 | 0.16 | 0.24 |
| 36 | 0.96 | 1.21 | 1.05 | 0.80 | 0.13 | 0.11 |
| 49 | 2.50 | 2.49 | 2.23 | 1.83 | 0.27 | 0.28 |
| 47 | 1.79 | 1.92 | 1.82 | 1.21 | 0.16 | 0.14 |
| 48 | 2.72 | 2.80 | 2.83 | 2.49 | 0.14 | 0.15 |
| 3 | 2.86 | 2.86 | 2.98 | 2.87 | 0.18 | 0.24 |
| 35 | 3.35 | 3.37 | 3.26 | 3.11 | 0.15 | 0.12 |
| 39 | 1.94 | 1.89 | 1.95 | 2.62 | 0.13 | 0.14 |
| 54 | 3.05 | 2.92 | 3.01 | 2.54 | 0.16 | 0.18 |
| 37 | 2.59 | 2.70 | 2.64 | 1.82 | 0.15 | 0.17 |
| 41 | 2.92 | 3.02 | 2.94 | 2.66 | 0.15 | 0.19 |
| 52 | 2.66 | 2.56 | 2.43 | 2.02 | 0.22 | 0.23 |
| 40 | 3.10 | 3.26 | 3.07 | 3.09 | 0.19 | 0.40 |
| 42 | 3.13 | 3.18 | 3.02 | 2.38 | 1.15 | 0.20 |
| 55 | 3.07 | 3.07 | 2.88 | 1.29 | 0.15 | 0.19 |
| 56 | 3.01 | 2.90 | 2.85 | 0.71 | 0.16 | 0.27 |
| 57 | 2.43 | 2.61 | 2.39 | 1.82 | 0.16 | 0.19 |
| 58 | 3.30 | 3.32 | 3.04 | 2.37 | 0.19 | 0.28 |
| 80 | 1.39 | 2.40 | 1.31 | 0.17 | 0.13 | 0.18 |
| 59 | 3.21 | 3.20 | 3.09 | 2.62 | 0.21 | 0.30 |
| 51 | 1.92 | 2.08 | 2.00 | 1.55 | 0.23 | 0.11 |
| 62 | 3.17 | 3.08 | 2.93 | 2.79 | 0.15 | 0.15 |
| 63 | 1.61 | 1.91 | 1.89 | 1.56 | 0.15 | 0.13 |
| 38 | 3.01 | 2.88 | 2.93 | 2.82 | 0.13 | 0.12 |
| 43 | 2.84 | 2.82 | 2.72 | 2.33 | 0.14 | 0.13 |
| 64 | 1.34 | 1.51 | 1.44 | 0.46 | 0.16 | 0.13 |
| 65 | 2.93 | 2.81 | 2.84 | 1.13 | 0.18 | 0.26 |
| 44 | 3.25 | 3.17 | 2.89 | 0.43 | 0.17 | 0.22 |

| | Neutralizing activity (%) | | | | | | | |
|---|---|---|---|---|---|---|---|---|
| | H1N1 | | | | H5N1 | | H3N2 | |
| | Bri07 | | Su109pdm | | Ind05 | | Pan99 | |
| Group | 250 | 100 | 250 | 100 | 250 | 100 | 250 | 100 |
| 1 | 98.0 | 96.0 | 100 | 100 | 52.9 | 67.6 | -0.7 | -0.7 |
| 2 | 100 | 100 | 97.7 | 85.3 | 97.1 | 90.6 | 34.2 | 3.4 |
| 36 | 94.1 | 84.2 | 85.9 | 97.7 | 52.9 | 50.6 | -2.7 | 7.5 |
| 49 | 100 | 95.1 | 83.6 | 93.0 | 69.4 | 91.8 | 91.8 | 54.8 |
| 47 | 100 | 100 | 100 | 97.7 | 49.4 | 29.4 | -6.8 | 1.4 |
| 48 | 100 | 98.0 | 100 | -18.3 | 60.8 | 54.6 | 19.9 | -0.7 |
| 3 | 100 | 98.0 | 100 | 100 | 85.9 | 81.8 | -8.9 | -11.6 |
| 35 | 100 | 100 | 100 | 100 | 95.9 | 94.1 | -4.8 | 3.4 |
| 39 | 64.5 | 48.7 | 100 | 95.3 | 41.2 | 31.2 | 15.8 | -2.7 |
| 54 | 100 | 96.1 | 94.2 | 100 | 80.7 | 66.4 | -11.0 | -0.7 |
| 37 | 100 | 100 | 100 | 93.0 | 97.1 | 90.0 | 5.5 | -8.9 |
| 41 | 92.1 | 96.1 | 100 | 100 | 85.9 | 81.2 | 40.4 | 3.4 |
| 52 | 100 | 100 | 100 | 100 | 93.2 | 86.9 | 5.5 | 9.6 |
| 40 | 100 | 98.0 | 100 | 100 | 97.6 | 94.1 | -6.8 | -8.9 |
| 42 | 100 | 100 | 97.7 | 100 | 98.2 | 96.5 | 34.2 | 15.8 |
| 55 | 96.1 | 96.1 | 69.5 | 88.3 | 22.4 | 2.4 | -6.8 | -17.1 |
| 56 | 92.1 | 84.2 | 27.9 | 13.5 | 1.7 | -9.5 | 7.5 | 7.5 |
| 57 | 92.1 | 84.2 | 83.8 | 60.2 | 61.2 | 30.0 | 13.7 | 11.0 |
| 58 | 100 | 100 | 54.8 | 76.6 | 31.8 | 28.8 | 11.6 | -6.8 |
| 80 | 74.3 | 70.4 | 7.7 | 1.9 | 15.4 | -3.3 | -21.2 | -6.9 |
| 59 | 100 | 100 | 85.6 | 88.5 | 5.4 | -2.7 | -6.6 | -6.8 |
| 51 | 98.0 | 98.0 | 100 | 100 | 85.9 | 72.9 | -11.0 | 3.4 |
| 62 | 92.1 | 92.1 | 91.3 | 97.1 | 83.9 | 40.2 | -8.9 | 13.7 |
| 63 | 100 | 100 | 100 | 100 | 100 | 98.8 | -8.9 | -11.0 |
| 38 | 100 | 100 | 97.7 | 100 | 85.3 | 67.1 | -0.7 | -2.7 |
| 43 | 100 | 100 | 100 | 100 | 98.8 | 97.6 | 13.7 | 11.6 |
| 64 | 92.1 | 86.2 | 81.3 | 83.6 | 47.1 | 40.6 | 7.5 | -2.7 |
| 65 | 98.0 | 96.1 | 19.2 | 13.5 | -3.9 | -0.8 | -6.8 | -13.0 |
| 44 | 100 | 100 | 97.7 | 95.3 | 56.5 | 48.8 | 15.8 | 3.4 |

The first three lines in Table 4 indicate the group number of clones isolated by screening, the number of clone types included in each group, and the number of the isolated clones. Germline genes used by the clones were identified by comparing the VH amino acid sequences of all of the germline genes with those of representative clones, and the obtained identity was defined by percentage. The amino acid sequences of the CDR3 regions were shown. The binding activities of the clones to 4 types of H1N1 strains (NC99, SI06, Bri07 and Cal09) and 1 type of H3N2 strain (Pan99) were detected by enzyme-linked immunosorbent assay (ELISA). The binding activity was indicated by the absorbance at 492 nm. The neutralizing activities of the Fab-PP-type antibodies at a concentration of 100 or 250 μg/ml on the strains H1N1, H5N1 and H3N2 were measured by a focus reduction method. The reduction rate was defined by percentage. The HI activities of the Fab-PP antibodies were measured with respect to the H1N1 strains (Bri07 and Cal09). The lowest concentration (μg/ml) of the Fab-PP-type antibody to inhibit hemagglutination was shown.

### <Analysis of Neutralization Kinetics of Antibody Obtained in the Present Invention>

### Measurement of Neutralization Kinetics of IgG-Type Antibody to Influenza Virus Strains

A purified, completely-humanized IgG antibody was adjusted to concentrations of 200 μg/mL and 100 μg/mL with MEM containing 0.4% BSA, and the 100 μg/mL solution was further subjected to four-step dilution. To this antibody solution, an equal amount of influenza virus containing approximately 100 focus forming units was added, and the obtained mixture was then subjected to a neutralization reaction at 37° C. for 1 hour. MDCK cells, which had been cultured in a 96-well plate, were washed with PBS(−), the neutralization reaction solution was then added by 30 μL/well to the MDCK cells. Thereafter, the obtained mixture was reacted at 37° C. for 1 hour. After that, the reaction solution was removed, and the cells were then washed with PBS(−) once. MEM containing 0.4% BSA was added by 50 μL/well to the resulting cells, and the obtained mixture was then cultured in the presence of CO2 at 37° C. for 16 hours. Thereafter, the culture solution was removed, and the resulting cells were fixed with ethanol and were then dried. The infected cells were stained by an enzyme antibody technique using a peroxidase—anti-peroxidase complex (PAP method). The number of the infected cells was counted, and the infection inhibition rate was then calculated.

### Influenza Virus Strains

The following virus strains were used in the measurement of neutralizing activity. The terms in the parentheses indicate abbreviations.

### H1N1; A/Bribane/59/2007 (Bri/07)

H1N1 2009 pandemic; A/Suita/1/2009 (Sui/09)

### H2N2; A/Okuda/1957 (OK/57)

H5N1; A/Indonesia/5/2005 (H5N1)/PR-IBCDC-RG2 (clade2.1) (Ind/05)

### H3N2; A/Panama/2007/1999 (Pan/99)

### Completely Humanized IgG Antibodies Subjected to Measurement of Neutralizing Activity

### F081-007, F083-115, F083-336, and F045-092

### (Concerning the Control Antibody F045-092 Used in the Present Example)

Approximately 3 L of component blood was collected from a human born in 1973 according to apheresis.

After production of a phage antibody library, the library was screened against the influenza virus strain H3N2, and as a result, an anti-HA (hemagglutinin) antibody clone was isolated.

This antibody exhibits an extremely strong neutralizing activity against a wide range of H3 virus strains from 1968 to 2011, and it is highly likely that the binding region has, as an epitope, the receptor binding pocket of a hemagglutinin head domain.

Moreover, this antibody also exhibits a neutralizing activity on H1, H2 and H5 virus strains. That is to say, this antibody is a versatile antibody capable of neutralizing a wide range of influenza viruses belonging to, at least, Group 1 and Group 2 (Patent Literature 3).

### Results

All of the three types of antibodies (F081-007, F083-115, and F083-336), which had been subjected to the measurement, exhibited a high neutralizing activity on the studied virus strains (H1N1, H2N2 and H5N1) belonging to Group 1, which was 90 to 900 times, at an IC50 value, higher than that of the F045-092 antibody.

In particular, the IC50 values of these three antibodies were 0.09-0.23 μg/mL with respect to the H1N1 pandemic strain (Sui/09), although the neutralizing activity of the F045-092 antibody on this pandemic strain was not detected.

Moreover, the F081-007 antibody exhibited an extremely high neutralizing activity on all of the strains, and particularly, it exhibited an IC50 value that was approximately 900 times higher than the F045-092 antibody on the H5N1 strain (Ind/05) (0.36 μg/mL).

Furthermore, the neutralizing activity of the F083-115 antibody on the H3N2 strain was detected, although the activity was weak (IC50 value: 225 μm/mL).

From these results, it was demonstrated that the antibody of the present invention has overwhelmingly strong neutralization kinetics, in particular, on Group 1, in comparison to the antibody described in Patent Literature 3 as a prior art (Table 5 and FIG. 2).

TABLE 5
Neutralization kinetics of complete human IgG anti-influenza virus antibodies

| | | Infection inhibition rate (%) | | | | | | | | | |
|---|---|---|---|---|---|---|---|---|---|---|---|
| Influenza virus | Antibody | Antibody concentration (μg/mL) | | | | | | | | | |
| strain | clone | 200.0 | 100.0 | 25.0 | 6.3 | 1.6 | 0.391 | 0.98 | 0.024 | 0.006 | 0.002 |
| Bri/07 (H1N1) | F081-007 | 100.0 | 100.0 | 100.0 | 90.1 | 82.4 | 59.2 | 36.3 | 27.9 | 9.9 | 0.0 |
| | F083-115 | 100.0 | 100.0 | 98.5 | 80.2 | 67.6 | 28.6 | 19.8 | 0.0 | 0.0 | 0.0 |
| | F083-336 | 100.0 | 100.0 | 97.3 | 90.5 | 72.1 | 42.4 | 32.4 | 11.1 | 7.3 | 2.3 |
| | F045-092 | 58.4 | 30.2 | 24.4 | 5.3 | 0.0 | 0.0 | 0.0 | 0.0 | 0.0 | 0.0 |
| Sui/09) | F081-007 | 100.0 | 100.0 | 100.0 | 100.0 | 96.8 | 83.1 | 49.8 | 45.3 | 32.0 | 29.3 |
| (H1N1 2009 pdm) | F083-115 | 100.0 | 100.0 | 100.0 | 99.5 | 94.5 | 82.2 | 53.0 | 25.6 | 5.1 | 7.4 |
| | F083-336 | 100.0 | 100.0 | 100.0 | 100.0 | 93.2 | 62.1 | 30.7 | 30.7 | 13.8 | 0.0 |
| | F045-092 | 15.1 | 16.1 | 0.0 | 6.0 | 0.0 | 4.2 | 0.0 | 4.2 | 0.0 | 0.0 |
| OK/57 (H2H2) | F081-007 | 98.9 | 98.9 | 98.2 | 92.6 | 79.0 | 37.2 | 16.5 | 0.0 | | |
| | F083-115 | 98.5 | 89.3 | 86.7 | 66.4 | 40.5 | 22.1 | 1.0 | 0.0 | | |
| | F083-336 | 98.9 | 85.9 | 86.7 | 81.5 | 39.8 | 19.9 | 2.5 | 0.0 | | |
| | F045-092 | 39.4 | 15.8 | 0 | 0 | 0 | 0 | 0 | 0 | | |
| Ind/05 (H5N1) | F081-007 | 100.0 | 100.0 | 100.0 | 100.0 | 85.1 | 55.3 | 10.2 | 7.0 | | |
| | F083-115 | 98.9 | 95.9 | 86.7 | 81.5 | 39.8 | 19.9 | 2.5 | 0.0 | | |
| | F083-336 | 98.5 | 89.3 | 86.7 | 66.4 | 40.5 | 22.1 | 1.0 | 0.0 | | |
| | F045-092 | 38.6 | 28.2 | 14.3 | 0.0 | 0.0 | 0.0 | 0.0 | 0.0 | | |
| Pan/99 (H3N2) | F081-007 | 0.0 | 0.0 | 0.0 | 0.0 | 0.0 | 0.0 | 0.0 | | | |
| | F083-115 | 43.8 | 19.0 | 0.0 | 0.0 | 0.0 | 0.0 | 0.0 | | | |
| | F083-336 | 0.0 | 0.0 | 0.0 | 0.0 | 0.0 | 4.8 | 2.9 | | | |
| | F045-092 | 99.5 | 99.0 | 96.7 | 42.9 | 31.4 | 1.9 | 0.0 | | | |

1. An antibody, which neutralizes H1 influenza virus and/or H5 influenza virus, wherein the antibody has a heavy chain variable region having CDRs consisting of a heavy chain first complementarity-determining region (VH CDR1), a heavy chain second complementarity-determining region (VH CDR2) and a heavy chain third complementarity-determining region (VH CDR3), which are described in any one of the following (1) to (29), and a light chain variable region having CDRs consisting of a light chain first complementarity-determining region (VL CDR1), a light chain second complementarity-determining region (VL CDR2) and a light chain third complementarity-determining region (VL CDR3), which are described in any one of the following (1) to (29):
(1) VH CDR1 of SEQ ID NO: 1, VH CDR2 of SEQ ID NO: 2, VH CDR3 of SEQ ID NO: 3, VL CDR1 of SEQ ID NO: 4, VL CDR2 of SEQ ID NO: 5, and VL CDR3 of SEQ ID NO: 6,
(2) VH CDR1 of SEQ ID NO: 7, VH CDR2 of SEQ ID NO: 8, VH CDR3 of SEQ ID NO: 9, VL CDR1 of SEQ ID NO: 10, VL CDR2 of SEQ ID NO: 11, and VL CDR3 of SEQ ID NO: 12,
(3) VH CDR1 of SEQ ID NO: 13, VH CDR2 of SEQ ID NO: 14, VH CDR3 of SEQ ID NO: 15, VL CDR1 of SEQ ID NO: 16, VL CDR2 of SEQ ID NO: 17, and VL CDR3 of SEQ ID NO: 18,
(4) VH CDR1 of SEQ ID NO: 19, VH CDR2 of SEQ ID NO: 20, VH CDR3 of SEQ ID NO: 21, VL CDR1 of SEQ ID NO: 22, VL CDR2 of SEQ ID NO: 23, and VL CDR3 of SEQ ID NO: 24,
(5) VH CDR1 of SEQ ID NO: 25, VH CDR2 of SEQ ID NO: 26, VH CDR3 of SEQ ID NO: 27, VL CDR1 of SEQ ID NO: 28, VL CDR2 of SEQ ID NO: 29, and VL CDR3 of SEQ ID NO: 30,
(6) VH CDR1 of SEQ ID NO: 31, VH CDR2 of SEQ ID NO: 32, VH CDR3 of SEQ ID NO: 33, VL CDR1 of SEQ ID NO: 34, VL CDR2 of SEQ ID NO: 35, and VL CDR3 of SEQ ID NO: 36,
(7) VH CDR1 of SEQ ID NO: 37, VH CDR2 of SEQ ID NO: 38, VH CDR3 of SEQ ID NO: 39, VL CDR1 of SEQ ID NO: 40, VL CDR2 of SEQ ID NO: 41, and VL CDR3 of SEQ ID NO: 42,
(8) VH CDR1 of SEQ ID NO: 43, VH CDR2 of SEQ ID NO: 44, VH CDR3 of SEQ ID NO: 45, VL CDR1 of SEQ ID NO: 46, VL CDR2 of SEQ ID NO: 47, and VL CDR3 of SEQ ID NO: 48,
(9) VH CDR1 of SEQ ID NO: 49, VH CDR2 of SEQ ID NO: 50, VH CDR3 of SEQ ID NO: 51, VL CDR1 of SEQ ID NO: 52, VL CDR2 of SEQ ID NO: 53, and VL CDR3 of SEQ ID NO: 54,
(10) VH CDR1 of SEQ ID NO: 55, VH CDR2 of SEQ ID NO: 56, VH CDR3 of SEQ ID NO: 57, VL CDR1 of SEQ ID NO: 58, VL CDR2 of SEQ ID NO: 59, and VL CDR3 of SEQ ID NO: 60,
(11) VH CDR1 of SEQ ID NO: 61, VH CDR2 of SEQ ID NO: 62, VH CDR3 of SEQ ID NO: 63, VL CDR1 of SEQ ID NO: 64, VL CDR2 of SEQ ID NO: 65, and VL CDR3 of SEQ ID NO: 66,
(12) VH CDR1 of SEQ ID NO: 67, VH CDR2 of SEQ ID NO: 68, VH CDR3 of SEQ ID NO: 69, VL CDR1 of SEQ ID NO: 70, VL CDR2 of SEQ ID NO: 71, and VL CDR3 of SEQ ID NO: 72,
(13) VH CDR1 of SEQ ID NO: 73, VH CDR2 of SEQ ID NO: 74, VH CDR3 of SEQ ID NO: 75, VL CDR1 of SEQ ID NO: 76, VL CDR2 of SEQ ID NO: 77, and VL CDR3 of SEQ ID NO: 78,
(14) VH CDR1 of SEQ ID NO: 79, VH CDR2 of SEQ ID NO: 80, VH CDR3 of SEQ ID NO: 81, VL CDR1 of SEQ ID NO: 82, VL CDR2 of SEQ ID NO: 83, and VL CDR3 of SEQ ID NO: 84,
(15) VH CDR1 of SEQ ID NO: 85, VH CDR2 of SEQ ID NO: 86, VH CDR3 of SEQ ID NO: 87, VL CDR1 of SEQ ID NO: 88, VL CDR2 of SEQ ID NO: 89, and VL CDR3 of SEQ ID NO: 90,
(16) VH CDR1 of SEQ ID NO: 91, VH CDR2 of SEQ ID NO: 92, VH CDR3 of SEQ ID NO: 93, VL CDR1 of SEQ ID NO: 94, VL CDR2 of SEQ ID NO: 95, and VL CDR3 of SEQ ID NO: 96,
(17) VH CDR1 of SEQ ID NO: 97, VH CDR2 of SEQ ID NO: 98, VH CDR3 of SEQ ID NO: 99, VL CDR1 of SEQ ID NO: 100, VL CDR2 of SEQ ID NO: 101, and VL CDR3 of SEQ ID NO: 102,
(18) VH CDR1 of SEQ ID NO: 103, VH CDR2 of SEQ ID NO: 104, VH CDR3 of SEQ ID NO: 105, VL CDR1 of SEQ ID NO: 106, VL CDR2 of SEQ ID NO: 107, and VL CDR3 of SEQ ID NO: 108,
(19) VH CDR1 of SEQ ID NO: 109, VH CDR2 of SEQ ID NO: 110, VH CDR3 of SEQ ID NO: 111, VL CDR1 of SEQ ID NO: 112, VL CDR2 of SEQ ID NO: 113, and VL CDR3 of SEQ ID NO: 114,
(20) VH CDR1 of SEQ ID NO: 115, VH CDR2 of SEQ ID NO: 116, VH CDR3 of SEQ ID NO: 117, VL CDR1 of SEQ ID NO: 118, VL CDR2 of SEQ ID NO: 119, and VL CDR3 of SEQ ID NO: 120,
(21) VH CDR1 of SEQ ID NO: 121, VH CDR2 of SEQ ID NO: 122, VH CDR3 of SEQ ID NO: 123, VL CDR1 of SEQ ID NO: 124, VL CDR2 of SEQ ID NO: 125, and VL CDR3 of SEQ ID NO: 126,
(22) VH CDR1 of SEQ ID NO: 127, VH CDR2 of SEQ ID NO: 128, VH CDR3 of SEQ ID NO: 129, VL CDR1 of SEQ ID NO: 130, VL CDR2 of SEQ ID NO: 131, and VL CDR3 of SEQ ID NO: 132,
(23) VH CDR1 of SEQ ID NO: 133, VH CDR2 of SEQ ID NO: 134, VH CDR3 of SEQ ID NO: 135, VL CDR1 of SEQ ID NO: 136, VL CDR2 of SEQ ID NO: 137, and VL CDR3 of SEQ ID NO: 138,
(24) VH CDR1 of SEQ ID NO: 139, VH CDR2 of SEQ ID NO: 140, VH CDR3 of SEQ ID NO: 141, VL CDR1 of SEQ ID NO: 142, VL CDR2 of SEQ ID NO: 143, and VL CDR3 of SEQ ID NO: 144,
(25) VH CDR1 of SEQ ID NO: 145, VH CDR2 of SEQ ID NO: 146, VH CDR3 of SEQ ID NO: 147, VL CDR1 of SEQ ID NO: 148, VL CDR2 of SEQ ID NO: 149, and VL CDR3 of SEQ ID NO: 150,
(26) VH CDR1 of SEQ ID NO: 151, VH CDR2 of SEQ ID NO: 152, VH CDR3 of SEQ ID NO: 153, VL CDR1 of SEQ ID NO: 154, VL CDR2 of SEQ ID NO: 155, and VL CDR3 of SEQ ID NO: 156,
(27) VH CDR1 of SEQ ID NO: 157, VH CDR2 of SEQ ID NO: 158, VH CDR3 of SEQ ID NO: 159, VL CDR1 of SEQ ID NO: 160, VL CDR2 of SEQ ID NO: 161, and VL CDR3 of SEQ ID NO: 162,
(28) VH CDR1 of SEQ ID NO: 163, VH CDR2 of SEQ ID NO: 164, VH CDR3 of SEQ ID NO: 165, VL CDR1 of SEQ ID NO: 166, VL CDR2 of SEQ ID NO: 167, and VL CDR3 of SEQ ID NO: 168, and
(29) VH CDR1 of SEQ ID NO: 169, VH CDR2 of SEQ ID NO: 170, VH CDR3 of SEQ ID NO: 171, VL CDR1 of SEQ ID NO: 172, VL CDR2 of SEQ ID NO: 173, and VL CDR3 of SEQ ID NO: 174.

2. The antibody according to claim 1, wherein the heavy chain variable region and the light chain variable region, respectively, consist of amino acid sequences having the amino acid sequence numbers described in any one of the following (1) to (29):
(1) SEQ ID NO: 176 and SEQ ID NO: 178,
(2) SEQ ID NO: 180 and SEQ ID NO: 182,
(3) SEQ ID NO: 184 and SEQ ID NO: 186,
(4) SEQ ID NO: 188 and SEQ ID NO: 190,
(5) SEQ ID NO: 192 and SEQ ID NO: 194,
(6) SEQ ID NO: 196 and SEQ ID NO: 198,
(7) SEQ ID NO: 200 and SEQ ID NO: 202,
(8) SEQ ID NO: 204 and SEQ ID NO: 206,
(9) SEQ ID NO: 208 and SEQ ID NO: 210,
(10) SEQ ID NO: 212 and SEQ ID NO: 214,
(11) SEQ ID NO: 216 and SEQ ID NO: 218,
(12) SEQ ID NO: 220 and SEQ ID NO: 222,
(13) SEQ ID NO: 224 and SEQ ID NO: 226,
(14) SEQ ID NO: 228 and SEQ ID NO: 230,
(15) SEQ ID NO: 232 and SEQ ID NO: 234,
(16) SEQ ID NO: 236 and SEQ ID NO: 238,
(17) SEQ ID NO: 240 and SEQ ID NO: 242,
(18) SEQ ID NO: 244 and SEQ ID NO: 246,
(19) SEQ ID NO: 248 and SEQ ID NO: 250,
(20) SEQ ID NO: 252 and SEQ ID NO: 254,
(21) SEQ ID NO: 256 and SEQ ID NO: 258,
(22) SEQ ID NO: 260 and SEQ ID NO: 262,
(23) SEQ ID NO: 264 and SEQ ID NO: 266,
(24) SEQ ID NO: 268 and SEQ ID NO: 270,
(25) SEQ ID NO: 272 and SEQ ID NO: 274,
(26) SEQ ID NO: 276 and SEQ ID NO: 278,
(27) SEQ ID NO: 280 and SEQ ID NO: 282,
(28) SEQ ID NO: 284 and SEQ ID NO: 286, and
(29) SEQ ID NO: 288 and SEQ ID NO: 290.

3. The antibody according to claim 1, which is an antibody fragment selected from the group consisting of Fab, Fab′, F(ab′)₂, a single chain antibody (scFv), a dimerized V region (Diabody), a disulfide stabilized V region (dsFv), and a peptide comprising CDR.

4. DNA encoding the antibody according to claim 1.

5. A recombinant vector comprising the DNA according to claim 4.

6. A transformed cell line obtained by introducing the recombinant vector according to claim 5 into a host cell.

7. A method for producing an antibody, which comprises culturing the transformed cell line according to claim 6 in a medium, then allowing the transformed cell line to generate and accumulate the antibody in a culture, and then collecting the antibody from the culture.

8. A pharmaceutical composition comprising the antibody according to claim 1.

9. A passive immunotherapeutic agent against influenza, which comprises the antibody according to claim 1.
